(19) United States
(12) Patent Application Publication
(10) Pub. No.: US 2006/0251619 A1
(43) Pub. Date: Nov. 9, 2006

(54) Modified interferon-gamma polypeptides and methods for using modified interferon-gamma polypeptides

(76) Inventors: Gilles Borrelly, Epinay-sous-Senart (FR); Thierry Guyon, Palaiseau (FR); Lila Drittanti, Vigneux-sur-Seine (FR)

Correspondence Address: FISH & RICHARDSON, PC, P.O. BOX 1022, MINNEAPOLIS, MN, 55440-1022, US

(21) Appl. No.: 11/429,100

(22) Filed: May 4, 2006

Related U.S. Application Data
(60) Provisional application No. 60/678,031, filed on May 4, 2005.
(60) Provisional application No. 60/733,835, filed on Nov. 4, 2005.

(51) Int. Cl. A61K 38/21; C12P 21/04; C40B 40/10; C07K 14/57

(52) U.S. Cl. 424/85.6; 530/351; 435/69.51; 435/320.1; 435/325

(57) ABSTRACT

Provided are modified interferon-gamma (IFN-γ) polypeptides and methods of generating modified interferon-gamma polypeptides. Also provided are methods of treatment using modified interferon-gamma polypeptides.

### RELATED APPLICATIONS

Benefit of priority is claimed to U.S. provisional application Ser. No. 60/678,031, filed May 4, 2005, entitled “MODIFIED INTERFERON-GAMMA POLYPEPTIDES AND METHODS OF USING MODIFIED INTERFERON-GAMMA POLYPEPTIDES,” to Gilles Borrelly, Thierry Guyon and Lila Drittanti; and to U.S. provisional application Ser. No. 60/733,835, filed Nov. 4, 2005, entitled “MODIFIED INTERFERON-GAMMA POLYPEPTIDES AND METHODS FOR USING MODIFIED INTERFERON-GAMMA POLYPEPTIDES,” to Gilles Borrelly, Thierry Guyon and Lila Drittanti. The subject matter of each of these applications is incorporated by reference in its entirety.

This application is related to International PCT Application Serial No. (Attorney Docket No. 17109-016W01/926PC), filed May 4, 2006, entitled “MODIFIED INTERFERON-GAMMA POLYPEPTIDES AND METHODS FOR USING MODIFIED INTERFERON-GAMMA POLYPEPTIDES” to Nautilus Biotech, Gilles Borrelly, Thierry Guyon, and Lila Drittanti, which also claims priority to U.S. Provisional Application Ser. No. 60/678,031 and to U.S. Provisional Application Ser. No. 60/733,835.

This application is related to U.S. application Ser. No 11/176,830, to Rene Gantier, Thierry Guyon, Manuel Vega and Lila Drittanti, entitled “RATIONAL EVOLUTION OF CYTOKINES FOR HIGHER STABILITY, THE CYTOKINES AND ENCODING NUCLEIC ACID MOLECULES”, filed Jul. 6, 2005 and published as U.S. Application No. US 2006-0020116, which is a continuation of U.S. application Ser. No. 10/658,834, to Rene Gantier, Thierry Guyon, Manuel Vega and Lila Drittanti entitled “RATIONAL EVOLUTION OF CYTOKINES FOR HIGHER STABILITY, THE CYTOKINES AND ENCODING NUCLEIC ACID MOLECULES”, filed Sep. 8, 2003 and published as U.S. Application No. US-2004-0132977-A1. This application also is related to U.S. application Ser. No. 11/196,067, to Rene Gantier, Thierry Guyon, Cruz Ramos Hugo, Manuel Vega and Lila Drittanti entitled “RATIONAL DIRECTED PROTEIN EVOLUTION USING TWO-DIMENSIONAL RATIONAL MUTAGENESIS SCANNING”, filed Aug. 2, 2005 and published as U.S. Application No. US-2006-0020396-A1, which is a continuation of U.S. application Ser. No. 10/658,355, to Rene Gantier, Thierry Guyon, Cruz Ramos Hugo, Manuel Vega and Lila Drittanti entitled “RATIONAL DIRECTED PROTEIN EVOLUTION USING TWO-DIMENSIONAL RATIONAL MUTAGENESIS SCANNING”, filed Sep. 8, 2003 and published as U.S. Application No. US 2005-0202438.

This application also is related to U.S. application Ser. No. 10/658,834, filed on Sep. 8, 2003, published as U.S. Application No. US-2004-0132977-A1, entitled “RATIONAL EVOLUTION OF CYTOKINES FOR HIGHER STABILITY, THE CYTOKINES AND ENCODING NUCLEIC ACID MOLECULES,” to Rene Gantier, Thierry Guyon, Manuel Vega, and Lila Drittanti; and to published International PCT Application WO 2004/022747, entitled, “RATIONAL EVOLUTION OF CYTOKINES FOR HIGHER STABILITY, THE CYTOKINES AND ENCODING NUCLEIC ACID MOLECULES,” to Rene Gantier, Thierry Guyon, Manuel Vega and Lila Drittanti. This application also is related to U.S. application Ser. No. 10/658,355, filed Sep. 8, 2003; and to International PCT Application WO 2004/022593, entitled “RATIONAL DIRECTED PROTEIN EVOLUTION USING TWO-DIMENSIONAL RATIONAL MUTAGENESIS SCANNING,” to Rene Gantier, Thierry Guyon, Cruz Ramos Hugo, Manuel Vega and Lila Drittanti.

The subject matter of each of the above-referenced related applications is incorporated by reference in its entirety.

### FIELD OF INVENTION

Modified interferon-gamma (interferon-γ; IFN-γ) polypeptides are provided. The IFN-γ polypeptides are modified to exhibit physical properties and activities that differ from unmodified and wild-type IFN-γ polypeptides. Nucleic acid molecules encoding these polypeptides also are provided. Also provided are methods of treatment and diagnosis using the modified interferon-γ polypeptides.

### BACKGROUND

The delivery of therapeutic proteins for clinical use is a major challenge to pharmaceutical science. Once in the blood stream, these proteins are constantly eliminated from circulation within a short time by different physiological processes, involving metabolism as well as clearance using normal pathways for protein elimination, such as (glomerular) filtration in the kidneys or proteolysis in blood. Once in the luminal gastrointestinal tract, these proteins are constantly digested by luminal proteases. The latter is often the limiting process affecting the half-life of proteins used as therapeutic agents in per-oral administration and either intravenous or intramuscular injection. The problems associated with these routes of administration of proteins are well known and various strategies have been used in attempts to solve them.

A protein family that has been the focus of much clinical work and effort to improve its administration and bio-assimilation is the cytokine family. A potent and therapeutically active cytokine is the human interferon-gamma. IFN-γ is produced in a variety of immune cells, such as activated T cells and NK cells. IFN-γ interacts with a specific receptor at the cell surface and activates signal transduction pathways that produce immunomodulatory effects of this cytokine.

IFN-γ has been approved for treatment of a variety of diseases including chronic granulomatous disease and malignant osteopetrosis. Hence IFN-γ, as well as many other cytokines, are important therapeutic agents. Naturally occurring variants can have undesirable side effects as well as the problems of administration, bioavailability and short half-life. Hence, there is a need to improve properties of IFN-γ for its use as a biotherapeutic agent. Therefore, among the objects herein, it is an object to provide modified IFN-γ polypeptides that have improved therapeutic properties.

### SUMMARY

Provided herein are modified interferon-γ polypeptides that exhibit increased protein stability and/or increased protein half-life manifested as increased resistance to proteases or increased stability due to resistance to denaturing agents, i.e. thermal stability, compared to an unmodified interferon-γ polypeptide. Modified interferon-γ polypeptides provided herein include human and non-human interferon-γ polypeptides.

Therapeutic use of interferon-γ is well established for humans and other animals. Because of its instability in the blood stream, as well as under storage conditions, therapy with IFN-γ can require frequent and repeated applications. The modified IFN-γ polypeptides provided herein are mutant variants of IFN-γ that display improved stability. These variants possess increased protein half-life, including increased stability in the bloodstream and/or under storage conditions. Such increased stability includes stability as assessed by resistance to blood, intestinal or any other proteases and/or increased thermal tolerance and/or tolerance to pH and/or other potentially denaturing and stability disrupting conditions.

Modified interferon-γ polypeptides provided herein that exhibit increased protein stability include IFN-γ polypeptides modified at one or more amino acid positions compared to an unmodified IFN-γ. Modified loci are identified with reference to a known unmodified IFN-γ polypeptide, such as a human IFN-γ polypeptide having a sequence of amino acids set forth in SEQ ID NO: 1 or 2. Modified IFN-γ polypeptides provided herein contain one or more amino acid replacements, deletions and/or insertions compared with the unmodified reference IFN-γ polypeptide.

Provided herein, are modified interferon-γ polypeptides containing one or more amino acid replacements in an unmodified IFN-γ polypeptide corresponding to any of amino acid positions 2, 5-12, 14-18, 24, 28-29, 31, 32, 35, 36, 39, 44, 45, 48, 51, 53-55, 57, 59, 60, 62, 63, 71-80, 83, 84, 89-99, 100, 101, 105, 106, 109, 115, 117, 122, 123, 125, 128, 131-134, 137-139, 142, 143 of a mature interferon-γ. Such modified interferon-γ polypeptides exhibit increased protein stability compared to the unmodified interferon-γ. In one embodiment, the mature interferon-γ has the sequence of amino acids set forth in SEQ ID NO: 1 or 2. In another embodiment, the modified IFN-γ polypeptide contains one or more amino acid replacements in an unmodified IFN-γ polypeptide corresponding to any of amino acid positions 2, 5-12, 14-18, 24, 28-29, 31, 32, 35, 36, 39, 44, 45, 48, 51, 53-55, 57, 59, 60, 62, 63, 71-80, 83, 84, 89-99, 100, 101, 105, 106, 109, 115, 117, 122, 123, 125, 128, 131-134, 137-139, 142, 143 so long as if position 5, 6, 12, 17, 24, 62, 71, 74, 75, 77, 78, 89, 93, 96, 105, or 106 is replaced, the replacing amino acid is not cysteine; if position 9 or 28 is replaced, the replacing amino acid is not glutamine or cysteine; if position 15, 83 or 90 is replaced, the replacing amino acid is not serine, cysteine, or threonine; if position 29 is replaced, the replacing amino acid is not phenylalanine, asparagine, tyrosine, glutamine, valine, alanine, Methionine, isoleucine, lysine, arginine, threonine, histidine, cysteine, or serine; if position 31 is replaced, the replacing amino acid is not histidine, aspartic acid, alanine, methionine, asparagine, threonine, arginine, serine, or cysteine; if position 18, 32, 55, 57, 60, 63, 84, 95, or 139 is replaced, the replacing amino acid is not valine; if position 48, 73, or 143 is replaced, the replacing amino acid is not asparagine; if position 97 or 122 is replaced, the replacing amino acid is not asparagine or cysteine; if position 100 is replaced, the replacing amino acid is not glutamine; if position 101 is replaced, the replacing amino acid is not phenylalanine, asparagines, glutamine, valine, alanine, methionine, isoleucine, lysine, glycine, arginine, threonine, histidine, cysteine, or serine; if position 109 is replaced, the replacing amino acid is not serine or threonine; and if position 133 is replaced, the replacing amino acid is not asparagine.

In one example, the modified interferon-γ polypeptides provided herein include amino acid replacement(s) in an unmodified IFN-γ polypeptide at one or more positions corresponding to any of the following amino acid positions: 2, 7, 8, 10, 11, 14, 16, 27, 35, 36, 39, 44, 45, 51, 53, 54, 59, 72, 76, 80, 91, 92, 94, 98, 115, 117, 123, 125, 128, 131, 132, 134, 137, 138 and 142 of a mature human interferon-γ polypeptide.

The modified IFN-γ polypeptides provided herein have amino acid replacement(s) corresponding to any one or more amino acid positions including any of positions Y2, D5, P6, Y7, V8, K9, E10, A11, E12, L14, K15, K16, Y17, F18, D24, D27, N28, G29, L31, F32, I35, L36, W39, D44, R45, M48, Q51, V53, S54, F55, F57, L59, F60, N62, F63, K71, S72, V73, E74, T75, I76, K77, E78, D79, M80, K83, F84, K89, K90, K91, R92, D93, D94, F95, E96, K97, L98, T99, N100, Y101, D105, L106, Q109, E115, I117, E122, L123, P125, K128, K131, R132, K133, R134, M137, L138, F139, R142 and R143 of a mature human interferon-γ polypeptide.

Provided herein are modified IFN-γ polypeptide having amino acid replacement(s) corresponding to any one or more of Y2H, Y2I, D5N, D5Q, P6A, P6S, Y7H, Y7I, Y7E, Y7D, Y7K, Y7R, Y7N, Y7Q, Y7S, Y7T, V8E, V8D, V8K, V8R, V8N, V8Q, V8S, V8T, K9N, E10Q, E10H, E10N, A11E, A11D, A11K, A11R, A11N, A11Q, A11S, A11T, E12Q, E12H, E12N, L14I, L14V, L14E, L14D, L14K, L14R, L14N, L14Q, L14S, L14T, K15N, K15Q, K16N, K16Q, Y17H, Y17I, Y17E, Y17D, Y17K, Y17R, Y17N, Y17Q, Y17S, Y17T, F18I, F18E, F18D, F18K, F18R, F18N, F18Q, F18S, F18T, D24N, D24Q, D27N, D27Q, N28S, G29P, L31I, L31V, F32I, I35E, I35D, I35K, I35R, I35N, I35Q, I35S, I35T, L36I, L36V, L36E, L36D, L36K, L36R, L36N, L36Q, L36S, L36T, W39H, W39S, D44N, D44Q, R45H, R45Q, M48E, M48D, M48K, M48R, M48Q, M48S, M48T, Q51E, Q51D, Q51K, Q51R, V53E, V53D, V53K, V53R, V53N, V53Q, V53S, V53T, S54E, S54D, S54K, S54R, S54N, S54Q, S54T, F55E, F55D, F55K, F55R, F55N, F55Q, F55S, F55T, F57I, F57E, F57D, F57K, F57R, F57N, F57Q, F57S, F57T, L59I, L59V, L59E, L59D, L59K, L59R, L59N, L59Q, L59S, L59T, F60I, F60E, F60D, F60K, F60R, F60N, F60Q, F60S, F60T, N62E, N62D, N62K, N62R, F63I, K71N, K71Q, S72E, S72D, S72K, S72R, S72N, S72Q, S72T, V73E, V73D, V73K, V73R, V73Q, V73S, V73T, E74Q, E74H, E74N, T75E, T75D, T75K, T75R, T75N, T75Q, T75S, I76E, I76D, I76K, I76R, I76N, I76Q, I76S, I76T, K77N, K77Q, E78Q, E78H, E78N, D79N, D79Q, M80E, M80D, M80K, M80R, M80N, M80Q, M80S, M80T, K83N, K83Q, F84I, F84E, F84D, F84K, F84R, F84N, F84Q, F84S, F84T, K89N, K89Q, K90N, K90Q, K91N, K91Q, R92H, R92Q, D93N, D93Q, D94N, D94Q, F95I, F95E, F95D, F95K, F95R, F95N, F95Q, F95S, F95T, E96Q, E96H, E96N, K97Q, L98I, L98V, L98E, L98D, L98K, L98R, L98N, L98Q, L98S, L98T, T99E, T99D, T99K, T99R, T99N, T99Q, T99S, N100A, D105N, D105Q, L106I, L106V, Q109E, Q109D, Q109K, Q109R, Q109N, E115Q, E115H, E115N, I117E, E117D, I117K, I117R, I117N, I117Q, I117S, I117T, E122Q, E122H, L123I, L123V, P125A, P125S, K128N, K128Q, K131N, K131Q, R132H, R132Q, K133Q, R134H, R134Q, M137I, M137V, L138I, L138V, F139I, R142H, R142Q, R143H, and R143Q.

In one example, the amino acid replacement(s) correspond to any one or more of Y2H, Y2I, D5N, D5Q, P6A, P6S, Y7H, Y7I, K9N, E10Q, E10H, E10N, E12Q, E12H, E12N, L14I, L14V, K15N, K15Q, K16N, K16Q, Y17H, Y17I, F18I, D24N, D24Q, D27N, D27Q, N28S, L31I, L31V, F32I, L36I, L36V, W39H, W39S, D44N, D44Q, R45H, R45Q, F57I, L59I, L59V, F60I, F63I, K71N, K71Q, E74Q, E74H, E74N, K77N, K77Q, E78Q, E78H, E78N, D79N, D79Q, K83N, K83Q, F84I, K89N, K89Q, K90N, K90Q, K91N, K91Q, R92H, R92Q, D93N, D93Q, D94N, D94Q, F95I, E96Q, E96H, E96N, K97Q, L98I, L98V, D105N, D105Q, L106I, L106V, E115Q, E115H, E115N, E122Q, E122H, L123I, L123V, P125A, P125S, K128N, K128Q, K131N, K131Q, R132H, R132Q, K133Q, R134H, R134Q, M137I, M137V, L138I, L138V, F139I, F139V, R142H, R142Q, R143H, and R143Q. In some instances, such modified IFN-γ polypeptides exhibit increased resistance to proteolysis.

In another example, the amino acid replacement(s) correspond to any one or more of P6A, P6S, Y7I, E10Q, E10H, K16Q, Y17H, Y17I, F18I, F60I, F63I, K71N, K71Q, D79N, K89N, K89Q, K90N, K90Q, E96H, E96N, K97Q, L98I, D105N, L106I, L106V, P125A, P125S, K128N, K128Q, R132H, R132Q, K133Q, R134H, R134Q, M137I, M137V, L138I, L138V, F139I, F139V, R142H, R142Q, R143H, and R143Q. In some instances, such modified IFN-γ polypeptides also exhibit an increased activity compared to an unmodified IFN-γ polypeptide.

In an additional example, the amino acid replacement(s) correspond to any one or more of F63I, K89N, K89Q, K90N, K90Q, E96Q, E96H, E96N, L98I, L98V, D105N, D105Q, L106I, L106V, P125A, P125S, K133Q, R134H, R134Q, M137V, L138V, F139I, F139V, R142H, R142Q, R143H, and R143Q. In a further example, the amino acid replacement(s) correspond to any one or more of F63I, K89N, K89Q, K90N, E96H, E96N, L98I, D105N, L106I, L106V, P125A, P125S, K133Q, R134H, R134Q, M137V, L138V, F139I, F139V, R142H, R142Q, R143H, and R143Q.

Also provided herein are modified IFN-γ polypeptides having amino acid replacement(s) corresponding to any one or more of Y7E, Y7D, Y7K, Y7R, Y7N, Y7Q, Y7S, Y7T, V8E, V8D, V8K, V8R, V8N, V8Q, V8S, V8T, A11E, A11D, A11K, A11R, A11N, A11Q, A11S, A11T, L14E, L14D, L14K, L14R, L14N, L14Q, L14S, L14T, Y17E, Y17D, Y17K, Y17R, Y17N, Y17Q, Y17S, Y17T, F18E, F18D, F18K, F18R, F18N, F18Q, F18S, F18T, I35E, I35D, I35K, I35R, I35N, I35Q, I35S, I35T, L36E, L36D, L36K, L36R, L36N, L36Q, L36S, L36T, M48E, M48D, M48K, M48R, M48N, M48Q, M48S, M48T, Q51E, Q51D, Q51K, Q51R, V53E, V53D, V53K, V53R, V53N, V53Q, V53S, V53T, S54E, S54D, S54K, S54R, S54N, S54Q, S54T, F55E, F55D, F55K, F55R, F55N, F55Q, F55S, F55T, F57E, F57D, F57K, F57R, F57N, F57Q, F57S, F57T, L59E, L59D, L59K, L59R, L59N, L59Q, L59S, L59T, F60E, F60D, F60K, F60R, F60N, F60Q, F60S, F60T, N62E, N62D, N62K, N62R, S72E, S72D, S72K, S72R, S72N, S72Q, S72T, V73E, V73D, V73K, V73R, V73N, V73Q, V73S, V73T, T75E, T75D, T75K, T75R, T75N, T75Q, T75S, I76E, I76D, I76K, I76R, I76N, I76Q, I76S, I76T, M80E, M80D, M80K, M80R, M80N, M80Q, M80S, M80T, F84E, F84D, F84K, F84R, F84N, F84Q, F84S, F84T, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, T99S, Q109E, Q109D, Q109K, Q109R, I117E, I117D, I117K, I117R, I117N, I117Q, I117S and I117. In some instances, such IFN-γ polypeptides exhibit increased conformational stability and are more resistant to denaturing agents such as temperature or pH.

In one example, the amino acid replacement(s) correspond to any one or more of Y7E, Y7D, Y7K, Y7R, Y7N, Y7Q, Y7S, Y7T, A11E, A11D, A11K, A11R, A11N, A11Q, A11S, A11T, L14E, L14D, L14K, L14R, L14N, L14Q, L14S, L14T, Y17E, Y17D, Y17K, Y17R, Y17N, Y17Q, Y17S, Y17T, L36E, L36D, L36K, L36R, L36N, L36Q, L36S, L36T, V53E, V53D, V53K, V53R, V53N, V53Q, V53S, V53T, F57E, F57D, F57K, F57R, F57N, F57Q, F57S, F57T, F60E, F60D, F60K, F60R, F60N, F60Q, F60S, F60T, S72E, S72D, S72K, S72R, S72N, S72Q, S72T, V73E, V73D, V73K, V73R, V73Q, V73S, V73T, T75E, T75D, T75K, T75R, T75N, T75Q, T75S, I76E, I76D, I76K, I76R, I76N, I76Q, I76S, I76T, M80E, M80D, M80K, M80R, M80N, M80Q, M80S, M80T, F84E, F84D, F84K, F84R, F84N, F84Q, F84S, and F84T. In some instances, such modified IFN-γ polypeptides exhibit increased stability due to increased intra-stability of the polypeptide monomer.

In another example, the amino acid replacement(s) correspond to any one or more of Y7E, Y7D, Y7K, Y7R, Y7N, Y7Q, Y7S, Y7T, V8E, V8D, V8K, V8R, V8N, V8Q, V8S, V8T, A11E, A11D, A11K, A11R, A11N, A11Q, A11S, A11T, L14E, L14D, L14K, L14R, L14N, L14Q, L14S, L14T, F18E, F18D, F18K, F18R, F18N, F18Q, F18S, F18T, I35E, I35D, I35K, I35R, I35N, I35Q, I35S, I35T, M48E, M48D, M48K, M48R, M48N, M48Q, M48S, M48T, Q51E, Q51D, Q51K, Q51R, S54E, S54D, S54K, S54R, S54N, S54Q, S54T, F55E, F55D, F55K, F55R, F55N, F55Q, F55S, F55T, L59E, L59D, L59K, L59R, L59N, L59Q, L59S, L59T, N62E, N62D, N62K, N62R, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, T99S, Q109E, Q109D, Q109K, Q109R, I117E, I117D, I117K, I117R, I117N, I117Q, I117S and I117T. In some instances, such modified IFN-γ polypeptides exhibit increased protein stability due to increased inter-stability of the polypeptide dimer.

In an additional example, the amino acid replacement(s) correspond to any one or more of N28A, N28K, G29P, M48E, M48D, M48K, M48R, M48N, M48Q, M48S, M48T, Q51E, Q51D, Q51K, Q51R, S54E, S54D, S54K, S54R, S54N, S54Q, S54T, F55E, F55D, F55K, F55R, F55N, F55Q, F55S, F55T, N62E, N62D, N62K, N62R, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, T99S, and N100A. In some instances, such modified IFN-γ polypeptides exhibit increased protein stability due to increased stability around glycosylation sites. In some examples, the modified IFN-γ polypeptide contains amino acid replacement(s) N28A and N100A. In other examples, the modified IFN-γ polypeptide contains amino acid replacement(s) N28K and G29P.

Provided herein is a modified interferon-γ polypeptides containing two or more amino acid replacements in an unmodified IFN-γ polypeptide corresponding to amino acid replacements of a mature interferon-γ polypeptide described herein. In one embodiment, an unmodified interferon-γ polypeptide has a sequence of amino acids set forth in SEQ ID NO: 1 or 2. In some instances, interferon-γ polypeptides so-modified exhibit increased protein stability compared to the unmodified interferon-γ polypeptide. Provided herein is a modified interferon-γ polypeptides wherein the number of amino acid positions replaced in an unmodified interferon-γ is 2, 3, 4, 5, 6, 7, 8, 9, 10, 11, 12, 13, 14, 15, 16, 17, 18, 19 or 20.

In one embodiment, the modified interferon-γ further includes a modification in one or more of positions corresponding to positions 5, 9, 28, 33, 37, 40, 41, 42, 58, 61, 64-66, 86, 88, 124, 128, 133 and 140 in a mature polypeptide having the sequence of amino acids set forth in SEQ ID NO: 1 or 2. In a particular embodiment, the further modifications include any of D5N, K9Q, N28S, N28A, N28H, L33I, L33V, K37N, K37Q, K40N, K40Q, E41H, E41N, E41Q, E42Q, E42H, E42N, K58N, K58Q, K61N, K61Q, K64N, K64Q, D65N, D65Q, D66N, D66Q, N86D, N88D, S124P, K128E, K133T and Q140R. In some examples, the modified interferon-γ further contain a modification in one or more positions to optimize one or more glycosylation sites.

Provided herein is a modified IFN-γ polypeptide that is a human IFN-γ polypeptide. In other examples, the modified IFN-polypeptide is a non-human IFN-polypeptide.

Also provided herein is a modified IFN-γ polypeptide that contains amino acid modifications in an unmodified IFN-γ polypeptide having the sequence of amino acids set forth in SEQ ID NO: 1 or SEQ ID NO:2. In some examples, the unmodified IFN-γ polypeptide has a length of 124, 125, 126, 127, 128, 129, 130, 131, 132, 133, 134, 135, 136, 137, 138, 139, 140, 141, 142, 143, 144, 145, 145, 146, 147, 148, 149, 150, 151, 152, 153, 154, 155, 156, 157, 158, 159, 160, 161, 162, 163, 164, 165, 166, 167, 168, 169 or 170 amino acids, or combinations thereof. In some instances, the modified IFN-γ polypeptide is heterogeneous in length or structure. In other instances, the modified IFN-γ polypeptide is homogenous in length or structure. In some examples, the modified IFN-γ polypeptide is 146 amino acids in length. In other examples, the modified IFN-γ polypeptide is 143 amino acids in length. In a further example, the modified IFN-γ polypeptide is 132 amino acids in length.

Provided herein are any of the above modified interferon-γ polypeptides, wherein the one or more amino acid replacements are selected from among natural amino acids, non-natural amino acids and a combination of natural and non-natural amino acids. In one example, the polypeptide exhibits increased protein stability by modification of only the primary sequence of the polypeptide. In one embodiment, the modified interferon-γ polypeptide is a naked polypeptide chain. In one embodiment, the modified interferon-γ polypeptide is a polypeptide complex wherein the interferon-γ polypeptide has been pegylated, albuminated, or glycosylated.

Provided herein are any of the above modified interferon-γ polypeptides, further containing one or more pseudo-wild type mutations. In one embodiment, the pseudo-wild-type mutations include, but are not limited to, one or more of insertions, deletions or replacements of the amino acid residue(s) of the unmodified interferon-γ polypeptide.

Provided herein are any of the above modified interferon-γ polypeptides where increased stability is manifested as an increased resistance to proteolysis. In one embodiment, the increased resistance to proteolysis occurs in serum, blood, saliva, digestive fluids or in vitro when exposed to proteases. The increased resistance to proteolysis is exhibited by the modified interferon-γ when it is administered intravenously, orally, nasally, pulmonarily, or is present in the digestive tract. Such modified interferon-γ polypeptides exhibit increased resistance to proteolysis by one or more proteases compared to the unmodified interferon-γ. Exemplary proteases include, but are not limited to, gelatinase A, gelatinase B, pepsin, trypsin, trypsin (Arg blocked), trypsin (Lys blocked), clostripain, endoproteinase Asp-N, chymotrypsin, cyanogen bromide, iodozobenzoate, Myxobacter P., Armillaria, luminal pepsin, microvillar endopeptidase, dipeptidyl peptidase, enteropeptidase and hydrolase.

Provided herein are any of the above modified interferon-γ polypeptides in which increased stability is manifested as increased thermal tolerance. In one embodiment, the modified interferon-γ has increased thermal tolerance at a temperature from about or at 20° C. to at or about 45° C. In a particular embodiment, the modified interferon-γ has increased thermal tolerance at a body temperature of a subject (e.g., about 37° C.).

Provided herein are any of the above modified interferon-γ polypeptides, in which the increased stability is manifested as an increased half-life in vivo or in vitro. In a particular embodiment, the increased stability is manifested as an increased half-life when administered to a subject. In one such embodiment, the modified interferon-γ has a half-life increased by at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% and at least 500% or more compared to the half-life of unmodified interferon-γ. In another such embodiment, the modified interferon-γ has a half-life increased by at least 1.5 times, 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times and 1000 times, or more times when compared to the half-life of unmodified interferon-γ.

Provided herein are any of the above modified interferon-γ polypeptides exhibiting increased activity compared to the unmodified interferon-γ. Provided herein are any of the above modified interferon-γ polypeptides exhibiting decreased activity compared to the unmodified interferon-γ. Activity can be assessed, for example, by measuring cell proliferation in vitro, measuring anti-viral activity in vitro or in vivo, or measuring binding to an interferon-γ receptor. The results of such assays correlate with an in vivo activity and hence a biological activity.

In one embodiment, the modified interferon-γ exhibits increased resistance to proteolysis and exhibits decreased thermal tolerance compared to the unmodified interferon-γ. Alternatively, in another embodiment, the modified interferon-γ exhibits increased thermal tolerance and exhibits decreased resistance to proteolysis compared to the unmodified interferon-γ.

Provided herein are any of the above modified interferon-γ polypeptides containing a signal peptide. In a particular embodiment, the signal sequence is amino acids 1-20 of the sequence of amino acids set forth in SEQ ID NOS: 370 and 371. Provided herein are any of the above modified interferon-γ polypeptides that do not have a signal peptide. In one embodiment, the modified interferon-γ polypeptides provided herein are secreted.

Also provided herein, are modified IFN-γ polypeptides produced in mammalian cells. In other instances, the modified IFN-γ polypeptides are produced in E. coli.

Provided herein are modified IFN-γ polypeptides that have only a single mutation in an unmodified native IFN-γ. In one example, the modified IFN-γ polypeptide has 2, 3, 4, 5, 6, 7, 8, 9, 10, 11, 12, 13, 14, 15, 16, 17, 18, 19, or 20 mutations in an unmodified native IFN-γ polypeptide.

Provided herein is a modified IFN-γ that is a dimer. In one example, the dimer is a homodimer. In another example, the dimer is a heterodimer where the monomeric IFN-γ polypeptide have a different sequence of amino acids. In some instances, the dimer is a fusion protein. In one example, the fusion protein contains two IFN-γ monomers linked directly or indirectly via a linker. In another example, the two monomers in the dimers are linked via non-covalent linkages. Also provided herein, is a modified IFN-γ polypeptide that is a monomer.

Provided herein are modified interferon-γ polypeptides containing any of the sequences of amino acids set forth in SEQ ID NOS: 3-11, 13-27, 29-32, 34-37, 41, 42, 45, 46, 55-59, 63-65, 69, 77-91, 93-105, 107-109, 111-113, 116-124, 126-135, 137-144, 146-217, 219-239, 241-350, 354-361, 368 and 369, or an active portion thereof.

It is understood that in all instances, modifications are with reference to SEQ ID NOS: 1 or 2 or for precursor forms with respect to SEQ ID NOS: 370 and 371. Corresponding loci on other IFN-γ polypeptides, including truncated variants, species of IFN-γ polypeptides and allelic variants, readily can be identified. Furthermore, shortened or lengthened variants with insertions or deletions of amino acids, particular at either terminus that retain an activity readily can be prepared and the loci for corresponding mutations identified.

In one embodiment is a modified cytokine structural homologue of a modified interferon-γ as described herein containing one or more amino acid replacements in the cytokine structural homologue at positions corresponding to the 3-dimensional-structurally-similar positions within the 3-D structure of the modified interferon-γ.

Provided herein are libraries (collections) of modified interferon-γ polypeptides containing two, three, four, five, six, 10, 50, 100, 200 or more modified interferon-γ polypeptides as described herein.

Provided herein are nucleic acid molecules containing a sequence of nucleic acids encoding a modified interferon-γ polypeptide as described herein. Provided herein are libraries (collections) of nucleic acid molecules comprising a plurality of the molecules as described herein.

Provided herein are vectors comprising the nucleic acid molecules. In one embodiment, the vectors are in a eukaryotic cell, a prokaryotic cell, an insect cell, a mammalian cell, etc. Also provided herein are libraries containing a plurality of the vectors.

Provided herein are methods for expressing a modified interferon-γ comprising: i) introducing a nucleic acid encoding a modified interferon-γ or a vector containing a nucleic acid encoding a modified interferon-γ into a cell, and ii) culturing the cell under conditions in which the encoded modified interferon-γ is expressed. In one embodiment, the vectors are in a eukaryotic cell, a prokaryotic cell, an insect cell, a mammalian cell, etc. In one embodiment, the modified interferon-γ is glycosylated.

Provided herein are pharmaceutical compositions including any of the modified interferon-γ polypeptides described herein in a pharmaceutically acceptable excipient, such as a binding agent, a filler, a lubricant, a disintegrant and a wetting agent.

In one embodiment, the pharmaceutical compositions are formulated for oral, nasal or pulmonary administration. In a particular embodiment, the pharmaceutical compositions are formulated for oral administration. In one embodiment, the modified interferon-γ in the pharmaceutical formulation exhibits increased half-life under conditions selected from exposure to saliva, exposure to proteases in the gastrointestinal tract and exposure to low pH conditions compared to wild-type cytokine. Proteases include, but are not limited to, one or more of a luminal pepsin, a microvillar endopeptidase, a dipeptidyl peptidase, an enteropeptidase and a hydrolase.

Provided in the pharmaceutical compositions herein are modified interferon-γ polypeptides, wherein the modification in the polypeptide includes removal of proteolytic digestion sites or modifications that confer increased stability of the protein structure. In one such embodiment, the modified interferon-γ in the pharmaceutical composition exhibits increased protein half-life or bioavailability in the gastrointestinal tract. Protein half-life can be increased in an amount of at least 10%, at least 20%, at least 30%, at least 40%, at least 50%, at least 50%, at least 60%, at least 70%, at least 80%, at least 90%, at least 100%, at least 150%, at least 200%, at least 250%, at least 300%, at least 350%, at least 400%, at least 450% or at least 500% or more compared to the half-life of wild-type protein. Alternatively, protein half-life can increased in an amount of at least 6 times, at least 7 times, at least 8 times, at least 9 times, at least 10 times, at least 20 times, at least 30 times, at least 40 times, at least 50 times, at least 60 times, at least 70 times, at least 80 times, at least 90 times, at least 100 times, at least 200 times, at least 300 times, at least 400 times, at least 500 times, at least 600 times, at least 700 times, at least 800 times, at least 900 times or at least 1000 times or more compared to wild-type protein.

Provided herein, protein half-life is increased in the presence of one or more proteases including, but not limited to, a luminal pepsin, a microvillar endopeptidase, a dipeptidyl peptidase, an enteropeptidase and a hydrolase. In one embodiment, the activity of the modified interferon-γ in the pharmaceutical composition is increased compared to the unmodified interferon-γ.

In one embodiment, protein half-life is increased after exposure to one or more conditions including, but not limited to, exposure to saliva, exposure to proteases in the gastrointestinal tract and exposure to low pH conditions.

Provided herein are pharmaceutical compositions prepared without the use of protease inhibitors, such as a Bowman-Birk inhibitor, a conjugated Bowman-Birk inhibitor, aprotinin and camostat.

Provided herein are pharmaceutical compositions formulated for oral administration in a form such as a liquid, a pill, a tablet or a capsule. In one embodiment, the pill or tablet is chewable. In one embodiment, the pill or tablet dissolves when exposed to saliva on the tongue or in the mouth. In an additional example, the capsule is a gastro-resistant capsule. In one embodiment, the pharmaceutical composition in the capsule is in liquid form. In an embodiment wherein the pharmaceutical composition is a liquid, the liquid is can be, for example, a solution, a syrup or a suspension. In another embodiment, the pharmaceutical composition in the capsule is in lyophilized form.

Provided herein are pharmaceutical compositions formulated for controlled release of the modified interferon-γ polypeptide. In one such example, the pharmaceutical composition is in the form of a tablet or a lozenge. Lozenges deliver the modified interferon-γ to the mucosa of the mouth, the mucosa of the throat or the gastrointestinal tract. Additionally, the lozenge can be formulated with an excipient, such as among anhydrous crystalline maltose and magnesium stearate.

Provided herein are pharmaceutical compositions formulated without protective compounds. In one such embodiment, the modified interferon-γ exhibits resistance to intestinal proteases including, but not limited to, a luminal pepsin, a microvillar endopeptidase, a dipeptidyl peptidase, an enteropeptidase and a hydrolase.

Pharmaceutical compositions can be further formulated with one or more pharmaceutically-acceptable additives, such as a suspending agent, an emulsifying agent, a non-aqueous vehicle, and/or a preservative.

Provided herein are pharmaceutical compositions of nucleic acid molecules encoding any of the modified interferon-γ polypeptides described herein or a vector containing a nucleic acid molecule encoding any of the modified interferon-γ polypeptides described herein and a pharmaceutically acceptable excipient.

Provided herein are methods of treating a subject exhibiting symptoms of or having interferon-γ-mediated disease or condition by administering any of the pharmaceutical compositions described herein. In one embodiment, the interferon-γ-mediated disease or condition includes, but is not limited to, a viral infection (e.g., hepatitis C, acquired immunodeficiency syndrome), a bacterial infection, a fungal infection (e.g., aspergillosis, candidemia), a cancerous condition (e.g., neutropenia, hemopoietic cell transplantation), a cancer, liver disease, a pulmonary disease or condition, malignant osteopetrosis or chronic granulomatous. In a particular embodiment, the subject to be treated is immunocompromised. In one embodiment, the symptoms of the subject are ameliorated or eliminated.

Provided herein are methods for producing a modified target protein, having an evolved predetermined property or activity, the method including the steps of: a) selecting, on a target protein, one or more is-HIT target amino acids amenable to providing the evolved predetermined property or activity upon amino acid replacement; wherein the is-HIT target amino acids are determined by identifying structurally homologous loci between the evolving target protein and a modified interferon-γ described herein possessing the predetermined property or activity; b) replacing each target amino acid with a replacement amino acid amenable to providing the evolved predetermined property or activity to form a candidate lead protein, wherein only one amino acid replacement occurs on each target protein; c) expressing from a nucleic acid molecule each candidate lead protein in a cell contained in an addressable array; and d) assaying each candidate LEAD protein to identify one or more proteins that have the predetermined property or activity that differs from an unmodified protein, thereby identifying evolved target proteins that are LEADs. In one embodiment, the method further includes the steps of: e) comparing the 3-dimensional structures of the evolving protein and the modified interferon-γ to identify regions of high coincidence between their backbones, the regions designated as structurally homologous regions; and f) identifying is-HIT structurally homologous loci on the evolving protein that correspond to structurally related is-HIT amino acid positions within a structurally homologous region of the modified interferon-γ. In one embodiment, the predetermined property or activity is protein stability, protein half life in vivo, thermal tolerance or resistance to proteolysis. Provided herein is a modified cytokine produced by the above method.

Provided herein are articles of manufacture including, but not limited to, packaging material and a pharmaceutical composition of a modified interferon-γ polypeptide described herein contained within the packaging material. In a particular embodiment, the pharmaceutical composition packaged within the article of manufacture is effective for treatment of an interferon-γ-mediated disease or disorder, and the packaging material includes a label that indicates that the modified interferon-γ is used for treatment of an interferon-γ-mediated disease or disorder.

Provided herein are kits including a pharmaceutical composition of a modified interferon-γ polypeptide as described herein, a device for administration of the modified interferon-γ polypeptide and optionally instructions for administration.

### DETAILED DESCRIPTION

DETAILED DESCRIPTION
Outline

| A. | Definitions | | | |
|---|---|---|---|---|
| B. | Interferon-γ | | | |
| | 1. | IFN-γ structure and function | | |
| | 2. | IFN-γ as a biopharmaceutical | | |
| C. | Exemplary methods for modifying IFN-γ | | | |
| | 1. | 1D Scanning (“Rational Mutagenesis”) | | |
| | 2. | 3D Scanning | | |
| | 3. | 2D-scanning (restricted rational mutagenesis) | | |
| | | a. | Identifying in-silico HITs | |
| | | b. | Identifying replacing Amino Acids | |
| | | c. | Construction of mutant proteins and biological assays | |
| D. | Modified Interferon-γ Polypeptides | | | |
| | 1. | Increased resistance to proteolysis by removal of proteolytic sites | | |
| | | a. | Proteases | |
| | | | i. | Serine Proteases |
| | | | ii. | Matrix Metalloproteinases |
| | | b. | Properties of IFN-γ variants by removal of proteolytic | |
| | | | sites | |
| | | c. | Generation of IFN-γ variants by removal of proteolytic | |
| | | | sites | |
| | | d. | Additional IFN-γ variants | |
| | | e. | Assessment of IFN-γ variants with increased resistance to | |
| | | | proteolysis | |
| | 2. | Increased Stability | | |
| | | a. | Properties of stable IFN-γ variants | |
| | | | i. | Creation of intra-molecular stability |
| | | | ii. | Increasing interactions between helices of the |
| | | | | dimer |
| | | | iii. | Stability in sites near glycosylation sites of IFN-γ |
| | | b. | Assessment of stability of IFN-γ variants | |
| | 3. | Super-LEADs and Additional IFN-γ modifications | | |
| | 4. | Further Modifications | | |
| E. | Production of modified interferon-γ polypeptides (variants) | | | |
| | 1. | Expression systems | | |
| | | a. | Prokaryotic expression | |
| | | b. | Yeast | |
| | | c. | Insects and insect cells | |
| | | d. | Mammalian cells | |
| | | e. | Plants | |
| | 2. | Purification | | |
| | 3. | Fusion Proteins | | |
| | 4. | Polypeptide modification | | |
| | 5. | Nucleotide sequences | | |
| F. | Assessing Interferon-γ variant activities | | | |
| | 1. | In vitro assays | | |
| | 2. | Non-human animal models | | |
| | 3. | Clinical Assays | | |
| G. | Formulation/Packaging/Administration | | | |
| | 1 | Administration of modified IFN-γ polypeptides | | |
| | 2. | Administration of nucleic acids encoding modified IFN-γ | | |
| | | polypeptides (gene therapy) | | |
| H. | Therapeutic Uses | | | |
| | 1. | Infections | | |
| | | a. | Viral infections | |
| | | | i. | Hepatitis C Virus |
| | | | ii. | Human immunodeficiency virus |
| | | | iii. | Herpesvirus |
| | | | iv. | Coxsackievirus B3 |
| | | b. | Bacterial infections | |
| | | | i. | Tuberculosis |
| | | | ii. | Leprocy |
| | | | iii. | NTM |
| | | c. | Fungal Infections | |
| | | | i. | Aspergillosis |
| | | | ii. | Candidiasis |
| | | | iii. | Pneumocystis jiroveci pneumonia |
| | | | iv. | Coccidioidomycosis |
| | | | V. | Histoplasmosis |
| | | | vi. | Cryptococcal meningitis |
| | | | vii. | Paracoccidioides brasiliensis |

| | d. | Parasitic Protozoa |
|---|---|---|
| 2. | Proliferative disorders | |
| | a. | Cancer |
| | b. | Osteopetrosis |
| | c. | Aterial conditions |
| 3. | Chronic Granulomatous Disease | |
| 4. | Idiopathic Pulmonary Fibrosis | |
| 5. | Hyper IgE states | |
| | a. | Atopic dermatitis |
| | b. | Asthma |
| | c. | Allergies |

| | |
|---|---|
| I. | Combination Therapies |
| J. | Articles of manufacture and Kits |
| K. | EXAMPLES |

A. Definitions

Unless defined otherwise, all technical and scientific terms used herein have the same meaning as is commonly understood by one of skill in the art to which the invention(s) belong. All patents, patent applications, published applications and publications, GenBank sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet or other sources. Reference thereto evidences the availability and public dissemination of such information.

As used herein, an “interferon-γ” polypeptide (also referred to herein as IFN-γ) refers to any interferon-γ polypeptide, including but not limited to, a recombinantly produced polypeptide, synthetically produced polypeptide, IFN-γ extracted from cells or tissues, such as from T-lymphocytes and Natural Killer cells and other sources. As isolated from any sources or as produced, mature IFN-γ polypeptides can be heterogeneous in length and typically range from 124 to 146 amino acids in length. Heterogeneity is observed at both termini. Generally, heterogeneity exists at the N-terminus due to post-translational removal of Cys-Tyr-Cys amino acids and at the C-terminus due to variable proteolytic processing. Heterogeneity also can result due to N-glycosylation of the polypeptide. Heterogeneity of IFN-γ polypeptides can differ depending on the source of the IFN-γ polypeptide. Hence reference to IFN-γ polypeptides refers to the heterogeneous population as produced or isolated. When a homogeneous preparation is intended, it will be so-stated. Reference to an IFN-γ polypeptide herein is to its monomeric or dimeric form, as appropriate.

Human IFN-γ (hIFN-γ) includes IFN-γ, allelic variant isoforms, synthetic molecules from nucleic acids, protein isolated from human tissue and cells, and modified forms thereof. Exemplary unmodified mature human IFN-γ polypeptides include, but are not limited to, unmodified and wild-type native IFN-γ polypeptide (such as the polypeptide containing a sequence set forth in SEQ ID NO: 1) and the unmodified and wild-type precursor IFN-γ polypeptide that includes a signal peptide (e.g., the polypeptide that has the sequence set forth in SEQ ID NO: 370), a polymorphic wild-type native IFN-γ polypeptide that has a mutation at Q140R (SEQ ID NO: 2; or its precursor containing a sequence of amino acids set forth in SEQ ID NO: 371). Other exemplary human IFN-γ polypeptides are those that are truncated at the N- or C-terminus as compared to SEQ ID NO:1 or SEQ ID NO:2, such as for example, polypeptides having a sequence of amino acids set forth in any one of SEQ ID NOS: 372-379.

Reference to IFN-γ polypeptide also includes allelic or species variants of IFN-γ, and truncated forms or fragments thereof. IFN-γ includes homologous polypeptides from different species including, but not limited to animals, including humans and non-human species, such as other mammals. As with human IFN-γ, non-human IFN-γ also includes heterogeneous lengths or fragments or portions of IFN-γ that are of sufficient length or include appropriate regions to retain at least one activity of full-length mature polypeptide.

Non-human IFN-γ polypeptides include IFN-γ polypeptides, allelic variant isoforms, synthetic molecules prepared from nucleic acids, protein isolated from non-human tissue and cells, and modified forms thereof. IFN-γ polypeptides of non-human origin include, but are not limited to, bovine, ovine, porcine, rat, rabbit, horse, other primates such as chimpanzees and macaques, pig, dog, mice and avian IFN-γ polypeptides. Exemplary precursor IFN-γ polypeptides of non-human origin include, for example, primates such as chimpanzees (Pan troglodytes, SEQ ID NO: 380) and macaques (Macaca fascicularis, SEQ ID NO: 381), pig (Sus scrofa, SEQ ID NO: 382), dog (Canis familiaris, SEQ ID NO: 383), horse (Equus caballus, SEQ ID NO: 384), bovine (Bos Taurus, SEQ ID NO: 385), mice (Mus musculus, SEQ ID NO: 386), common squirrel monkey (Saimiri sciureus, SEQ ID NO: 387), olive baboon (Papio anubis, SEQ ID NO: 388), giant panda (Ailuropoda melanoleuca, SEQ ID NO: 389), white-tufted ear marmoset (Callithrix jacchus, SEQ ID NO: 390), gorilla (Gorilla gorilla, SEQ ID NO: 391), Bactrian camel (Camelus bactrianus, SEQ ID NO: 392), red deer (Cervus elaphus, SEQ ID NO: 393), llama (Lama glama, SEQ ID NO: 394), rabbit (Oryctolagus cuniculus; SEQ ID NO: 395), bottle-nosed dolphin (Tursiops truncates, SEQ ID NO: 396), cat (Felis catus, SEQ ID NO: 397), sheep (Ovis aries, SEQ ID NO: 398) and golden hamster (Mesocricetus auratus, SEQ ID NO: 399). Each of the noted polypeptides as set forth, except for the chimpanzee, includes a signal peptide.

As used herein, an IFN-γ dimer refers to a combination of two monomeric IFN-γ polypeptides having the same or a different number of amino acids and/or different sequence of amino acids. Typically, the dimeric form of the polypeptide include those that contain two monomers linked via non-covalent interactions, including hydrophobic interactions, hydrogen bonds, van der Waals and other such interactions. Such dimers can form spontaneously when expressed and typically form spontaneously, such as, for example, as occurs using the methods of protein production described herein. Dimers also can be produced as fusion proteins, such as in the form of a single chain dimeric IFN-γ polypeptide due to direct or indirect linkage of the same or different monomers. For purposes herein, the first monomer of a dimer is designated “A” and the second monomer of the dimer is designated “B,” whereby the six helices of each monomer are designated A1-A6 and B1-B6, respectively.

As used herein, an allelic variant or allelic variation references a polypeptide encoded by a gene that differs from a reference form of a gene (i.e. is encoded by an allele) among a population. Typically the reference form of the gene encodes a wildtype form and/or predominant form of a polypeptide from a population or single reference member of a species. Typically, allelic variants, have at least 80%, 90%, 95% or greater amino acid identity with a wildtype and/or predominant form from the same species.

As used herein, species variants refers to variants of the same polypeptide between and among species. Generally, interpecies allelic variants have at least about 60%, 70%, 80%, 85%, 90% or 95% identity or greater with a wildtype and/or predominant form from another species, including 96%, 97%, 98%, 99% or greater identity with a wildtype and/or predominant form of a polypeptide.

As used herein, “native IFN-γ” refers to an interferon-γ polypeptide encoded by a naturally occurring IFN-γ gene, i.e. an IFN-γ gene that is present in an organism in nature, such as in an animal, including a human or other mammal. Included among native IFN-γ polypeptides are the encoded precursor polypeptide, fragments thereof, and processed forms thereof, such as a mature form lacking the signal peptide as well as any pre- or post-translationally processed or modified form thereof. For example, humans express IFN-γ. Exemplary of a native human IFN-γ polypeptide is the precursor IFN-γ containing the signal peptide (i.e. SEQ ID NO:370) as well as a mature IFN-γ polypeptide lacking the signal peptide (i.e. SEQ ID NO:1). Also included among native IFN-γ polypeptides are those that are post-translationally modified, including those that have a deletion of the Cys-Tyr-Cys N-terminal amino acids as compared to SEQ ID NO:1, those that are proteolytically processed at the C-terminus, and those that include other post-translational modifications such as, for example, glycosylation. Other animals, such as mammals, express native IFN-γ, and include, but are not limited to, hamsters, mice, cows, monkeys, orangutans, baboons, chimpanzees, macaques, gibbons, gorillas, red deer, llamas, rabbits, dolphins, cats, giant pandas and dogs. As noted above, in nature, the polypeptides occur as a heterogeneous mixture that contains polypeptides of varying lengths and epigenetic modification, such as differences in glycosylation patterns.

As used herein, a “portion or fragment of an IFN-γ polypeptide” or “an active portion” refers to any portion of a human or non-human IFN-γ polypeptide that exhibits one or more activities of the full-length polypeptide. Such activities include, for example, anti-viral, antimicrobial, anti-fungal, anti-tumoricidal, anti-fibrotic or anti-proliferative activities. Typically, a portion or fragment of an IFN-γ polypeptide provided herein that exhibits an activity of IFN-γ contains amino acids 24 to 149 of an unprocessed IFN-γ polypeptide, such as for example, corresponding to amino acids 24 to 149 of a precursor human IFN-γ polypeptide set forth in SEQ ID NO:370 or 371.

As used herein, an “activity” of an interferon-γ polypeptide refers to any activity exhibited by an interferon-γ polypeptide. Such activities can be tested in vitro and/or in vivo and include, but are not limited to, anti-viral, antimicrobial, anti-fungal, anti-tumoricidal, anti-fibrotic or anti-proliferative activity. Activity can be any level of percentage of activity of the polypeptide, including but not limited to, 1% of the activity, 2%, 3%, 4%, 5%, 10%, 20%, 30%, 40%, 50%, 60%, 70%, 80%, 90%, 95% or more of functional activity compared to the full-length polypeptide. For example, percentage of activity of the polypeptide also includes 96%, 97%, 98%, 99%, 100%, 200%, 300%, 400%, 500%, or more of functional activity compared to the full-length polypeptide. Activities can be measured in vitro or in vivo using recognized assays. The results of such assays that indicate that a polypeptide exhibits an activity can be correlated to activity of the polypeptide in vivo, which in vivo activity can be referred to as biological activity. Assays to determine functionality or activity of modified forms of IFN-γ are known to those of skill in the art. Assays include, for example, a cytopathic effects (CPE) assay, which is used to assess anti-viral activity of modified IFN-γ polypeptides. In one such assay, anti-viral activity of an IFN-γ polypeptide is assessed by determining the capacity of the modified IFN-γ polypeptide to protect HeLa cells against EMC (mouse encephalo-myocarditis) virus-induced cytopathic effects compared to an unmodified IFN-γ polypeptide. Anti-proliferative activity of an IFN-γ polypeptide can be determined by assessing the capacity of a modified IFN-γ polypeptide to inhibit proliferation of Daudi cells compared to an unmodified IFN-γ polypeptide. Daudi cells are seeded on plates and modified and unmodified IFN-γ polypeptides are added. After culturing, substrate is added and colorimetric changes are observed.

As used herein, “exhibits at least one activity” or “retains at least one activity” refers to the activity exhibited by a modified IFN-γ polypeptide as compared to an unmodified IFN-γ polypeptide. Generally, a modified IFN-γ polypeptide that retains an activity of an unmodified IFN-γ polypeptide either improves or maintains the requisite biological activity (e.g., anti-viral and anti-proliferation activity) of an unmodified IFN-γ polypeptide. In some instances, a modified IFN-γ polypeptide can retain an activity that is increased compared to an unmodified IFN-γ polypeptide. In some cases, a modified IFN-γ polypeptide can retain an activity that is decreased compared to an unmodified IFN-γ polypeptide. Activity of a modified polypeptide can be any level of percentage of activity of the unmodified polypeptide, including but not limited to, 1% of the activity, 2%, 3%, 4%, 5%, 10%, 20%, 30%, 40%, 50%, 60%, 70%, 80%, 90%, 100%, 200%, 300%, 400%, 500%, or more of functional activity compared to the unmodified polypeptide. For example, a modified IFN-γ polypeptide retains at least 1%, 2%, 3%, 4%, 5%, 6%, 7%, 8%, 9%, 10%, . . . 20%, . . . 30%, . . . 40%, . . . 50%, . . . 60%, . . . 70% . . . 80%, . . . 90%, . . . 95%, 96%, 97%, 98% or at least 99% of the activity of the wild-type protein. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than unmodified IFN-γ. Activity can be measured, for example, using assays such as those described in the Examples below.

As used herein, a recitation that a modified protein has more anti-viral activity (or other activity) than anti-proliferative activity (or another activity) compared to the unmodified cytokine is comparing the absolute value of the change in each activity compared to an unmodified or native form.

As used herein, a “property” of an IFN-γ polypeptide refers to any property exhibited by an IFN-γ polypeptide. Such properties include, but are not limited to, protein stability such as manifested by, for example, resistance to proteolysis, thermal tolerance, or tolerance to pH conditions. Changes in properties can alter an activity of the polypeptide. For example, thermal tolerance of an IFN-γ polypeptide can be determined by assessing the capacity of a modified IFN-γ polypeptide to retain anti-viral or anti-proliferative activity after exposure to increases in temperature. Supernatants containing modified or unmodified IFN-γ polypeptides are incubated/cultured at 37° C. for up to 60 hours. Aliquots are taken and tested for anti-viral and/or anti-proliferative activities using the methods described herein. In another example, the resistance of the modified IFN-γ polypeptides compared to unmodified wild-type IFN-γ against enzymatic cleavage by proteases can be empirically tested by treating the polypeptides with proteases and then testing the polypeptides for residual anti-viral and anti-proliferative activities.

As used herein, “EC50” refers to the effective concentration of IFN-γ necessary to give one-half of a maximum response. For purposes herein, the response measured is an activity of IFN-γ, such as but not limited to, activity in an anti-viral activity assay.

As used herein, “half-life” refers to the time required for a measured parameter, such as the potency, activity and effective concentration of a polypeptide, molecule to fall to half of its original level, such as half of its original potency, activity, or effective concentration at time zero. Thus, the parameter, such as potency, activity, or effective concentration of a polypeptide molecule is generally measured over time. For purposes herein, half-life can be measured in vitro or in vivo. For example, the half-life of IFN-γ or a modified IFN-γ polypeptide can be measured in vitro by assessing its activity (i.e. anti-viral activity) following incubation over increasing time under certain conditions, such as for example, after exposure to proteases, or denaturing conditions such as temperature of pH. In another example, the half-life of IFN-γ or a modified IFN-γ polypeptide can be measured in vivo following administration (i.e. intravenous, subcutaneous, intraduodenal, oral) of the polypeptide to a human or other animal, followed by sampling of the blood over time to determine the remaining effective concentration and/or activity of the polypeptide in the blood sample.

Increased protein half-life refers to a change in half-life of a modified polypeptide compared to an unmodified polypeptide. For example, a modified IFN-γ polypeptide exhibits increased half-life compared to an unmodified IFN-γ polypeptide when the time required for a measured parameter (i.e. anti-viral activity) to fall to one-half of the original level is greater than for the unmodified IFN-γ polypeptide. A modified polypeptide that exhibits increased half-life in vitro or in vivo exhibits, for example at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% and at least 500% or more half-life compared to the half-life of an unmodified interferon-γ. Half-life of a modified interferon-γ also can be increased by at least 1.5 times, 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times and 1000 times, or more times when compared to the half-life of an unmodified interferon-γ.

As used herein, “protein stability” refers to protein-half-life. Increased protein stability refers to a change in a property or properties of a protein that are manifested as increased half-life. Such changes include increased resistance, increased resistance to digestion by one or more proteases, increased resistance to denaturing conditions, such as, but not limited to, increased temperature, particular pH conditions and/or exposure to denaturing ingredients. A modified polypeptide that exhibits increased protein stability in vitro or in vivo is, for example, 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, or 100% more stable than an unmodified polypeptide. In some instances, a modified polypeptide is 105%, 110%, 120%, 130%, 140%, 150%, 200%, 300%, 400%, 500%, or more stable than an unmodified polypeptide.

As used herein, “serum stability” refers to protein stability in serum.

As used herein, “resistance to proteolysis” refers to any amount of decreased cleavage of a target amino acid residue of a modified polypeptide by a protease compared to cleavage of an unmodified polypeptide by the same protease under the same conditions. A modified polypeptide that exhibits increased resistance to proteolysis exhibits, for example, 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, or 100% more resistance to proteolysis than an unmodified polypeptide. In some instances, a modified polypeptide exhibits 105%, 110%, 120%, 130%, 140%, 150%, 200%, 300%, 400%, 500%, or more increased resistance to proteolysis compared to an unmodified polypeptide.

As used herein, “thermal tolerance” refers to any amount of decreased degradation after exposure to altered temperatures. A modified polypeptide that exhibits increased thermal tolerance exhibits, for example, 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, or 100% more stability at varied temperatures than an unmodified polypeptide. In some instances, a modified polypeptide exhibits 105%, 110%, 120%, 130%, 140%, 150%, 200%, 300%, 400%, 500%, or more stability at varied temperatures than an unmodified polypeptide. For example, a modified polypeptide exhibits increased thermal tolerance in vivo when administered to a subject than an unmodified polypeptide.

As used herein, “proteases,” “proteinases” or “peptidases” are interchangeably used to refer to enzymes that catalyze the hydrolysis of covalent peptidic bonds. Proteases include, for example, serine proteases and matrix metalloproteinases. Serine protease or serine endopeptidases constitute a class of peptidases, which are characterized by the presence of a serine residue in the active center of the enzyme. Serine proteases participate in a wide range of functions in the body, including blood clotting, inflammation as well as digestive enzymes in both prokaryotes and eukaryotes. The mechanism of cleavage by “serine proteases,” is based on nucleophilic attack of a targeted peptidic bond by a serine. Cysteine, threonine or water molecules associated with aspartate or metals also can play this role. Aligned side chains of serine, histidine and aspartate form a catalytic triad common to most serine proteases. The active site of serine proteases is shaped as a cleft where the polypeptide substrate binds. Amino acid residues are labeled from N to C termini of a polypeptide substrate (Pi, . . . , P3, P2, P1, P1′, P2′, P3′, . . . , Pj). The respective binding sub-sites are labeled (Si, . . . , S3, S2, S1, S1′, S2′, S3′, . . . , Sj). The cleavage is catalyzed between P1 and P1′.

As used herein, a matrix metalloproteinases (MMP) refers to any of a family of metal-dependent, such as Zn⁺²-dependent, endopeptidases that degrade components of the extracellular matrix (ECM). MMPs include four classes: collagenases, stromelysin, membrane-type metalloproteinases and gelatinases. Proteolytic activities of MMPs and plasminogen activators, and their inhibitors, are important for maintaining the integrity of the ECM. Cell-ECM interactions influence and mediate a wide range of processes including proliferation, differentiation, adhesion and migration of a variety of cell types. MMPs also process a number of cell-surface cytokines, receptors and other soluble proteins and are involved in tissue remodeling processes such as wound healing, pregnancy and angiogenesis. Under physiological conditions in vivo, MMPs are synthesized as inactive precursors (zymogens) and are cleaved to produce an active form. Additionally, the enzymes are specifically regulated by endogenous inhibitors called tissue inhibitors of matrix metalloproteinases (TIMPs).

As used herein, “a directed evolution method” refers to methods that “adapt” either proteins, including natural proteins, synthetic proteins or protein domains to have changed proportions, such as the ability to act in different or existing natural or artificial chemical or biological environments and/or to elicit new functions and/or to increase or decrease a given activity, and/or to modulate a given feature. Exemplary directed evolution methods include, among others, rational directed evolution methods described in U.S. application Ser. No. 10/022,249; and U.S. Published Application No. US-2004-0132977-A1.

As used herein, “two dimensional rational mutagenesis scanning (2-D scanning)” refers to the processes provided herein in which two dimensions of a particular protein sequence are scanned: (1) one dimension is to identify specific amino acid residues along the protein sequence to replace with different amino acids, referred to as is-HIT target positions, and (2) the second dimension is the amino acid type selected for replacing the particular is-HIT target, referred to as the replacing amino acid.

As used herein, “in silico” refers to research and experiments performed using a computer. In silico methods include, but are not limited to, molecular modeling studies and biomolecular docking experiments.

As used herein, “is-HIT” refers to an in silico identified amino acid position along a target protein sequence that has been identified based on i) the particular protein properties to be evolved, ii) the protein's sequence of amino acids, and/or iii) the known properties of the individual amino acids. These is-HIT loci on the protein sequence are identified without use of experimental biological methods. For example, once the protein feature(s) to be optimized is (are) selected, diverse sources of information or previous knowledge (i.e., protein primary, secondary or tertiary structures, literature, patents) are exploited to determine those amino acid positions that are amenable to improved protein fitness by replacement with a different amino acid. This step uses protein analysis “in silico.” All possible candidate amino acid positions along a target protein's primary sequence that might be involved in the feature being evolved are referred to herein as “in silico HITs” (“is-HITs”). The collection (library), of all is-HITs identified during this step represents the first dimension (target residue position) of the two-dimensional scanning methods provided herein.

As used herein, “amenable to providing the evolved predetermined property or activity” in the context of identifying is-HITs refers to an amino acid position on a protein that is contemplated, based on in silico analysis, to possess properties or features that when replaced result in the desired activity being evolved. The phrase “amenable to providing the evolved predetermined property or activity” in the context of identifying replacement amino acids refers to a particular amino acid type that is contemplated, based on in silico analysis, to possess properties or features that when used to replace the original amino acid in the unmodified starting protein result in the evolution of a desired or preselected activity.

As used herein, “high-throughput screening” (HTS) refers to processes that test a large number of samples, such as samples of test proteins or cells containing nucleic acids encoding the proteins of interest to identify structures of interest or to identify test compounds that interact with the variant proteins or cells containing them. HTS operations are amenable to automation and are typically computerized to handle sample preparation, assay procedures and the subsequent processing of large volumes of data.

As used herein, the term “restricted,” in the context of the identification of is-HIT amino acid positions along the amino acid residues in a protein selected for amino acid replacement and/or the identification of replacing amino acids, means that fewer than all amino acids on the protein-backbone are selected for amino acid replacement and/or fewer than all of the remaining 19 amino acids available to replace the original amino acid present in the unmodified starting protein are selected for replacement. In particular embodiments of the methods provided herein, the is-HIT amino acid positions are restricted such that fewer than all amino acids on the protein-backbone are selected for amino acid replacement. In other embodiments, the replacing amino acids are restricted such that fewer than all of the remaining 19 amino acids available to replace the native amino acid present in the unmodified starting protein are selected as replacing amino acids. In an exemplary embodiment, both of the scans to identify is-HIT amino acid positions and the replacing amino acids are restricted such that fewer than all amino acids on the protein-backbone are selected for amino acid replacement and fewer than all of the remaining 19 amino acids available to replace the native amino acid are selected for replacement.

As used herein, “candidate LEADs” are mutant proteins that are contemplated as potentially having an alteration in any attribute, chemical, physical or biological property or activity in which such alteration is sought. In the methods herein, candidate LEADs are generally generated by systematically replacing is-HITS loci in a protein or a domain thereof with typically a restricted subset, or all, of the remaining 19 amino acids, such as obtained using PAM analysis. Candidate LEADs can be generated by other methods known to those of skill in the art tested by the high throughput methods herein.

As used herein, “LEADs” are “candidate LEADs” whose activity has been demonstrated to be optimized or improved for the particular attribute, chemical, physical or biological property. For purposes herein a “LEAD” typically has activity with respect to the function of interest that differs by at least 5%, 10%, 20%, 30%, 40%, 50%, 60%, 70%, 80%, 90%, 100%, 150%, 200% or more from the unmodified and/or wild type (native) protein. For example, the activity of interest differs at least 10%, 50%, 100%, 200%, 300%, 400%, 500%, or more from the unmodified and/or wildtype protein. In certain embodiments, the change in activity is at least about 5%, 10%, 15%, 20%, 25%, 30%, 35%, 40%, 45%, 50%, 55%, 60%, 65%, 70%, 75%, 80%, 85%, 90%, 95% or 100%, of the activity of the unmodified target protein. In other embodiments, the change in activity is not more than about 5%, 10%, 15%, 20%, 25%, 30%, 35%, 40%, 45%, 50%, 55%, 60%, 65%, 70%, 75%, 80%, 85%, 90%, 95% or 100% of the activity of the unmodified target protein. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than the activity of the unmodified target protein. The desired alteration, which can be either an increase or a reduction in activity, depends upon the function or property of interest (e.g., ˜10%, ˜20%, etc.). The LEADs can be further optimized by replacement of a plurality (2 or more) of “is-HIT” target positions on the same protein molecule to generate “super-LEADs.”

As used herein, the term “super-LEAD” refers to protein mutants (variants) obtained by adding the single mutations present in two or more of the LEAD molecules in a single protein molecule. Accordingly, in the context of the modified proteins provided herein, the phrase “proteins comprising one or more single amino acid replacements” encompasses addition of two or more of the mutations described herein for one respective protein. For example, the modified proteins provided herein containing one or more single amino acid replacements can have any of 2, 3, 4, 5, 6, 7, 8, 9, 10, 11, 12, 13, 14, 15, 16, 17, 18, 19, 20 or more of the amino acid replacements at the disclosed replacement positions. The collection of super-LEAD mutant molecules is generated, tested and phenotypically characterized one-by-one in addressable arrays. Super-LEAD mutant molecules are molecules containing a variable number and type of LEAD mutation. Those molecules displaying further improved fitness for the particular feature being evolved, are referred to as super-LEADs. Super-LEADs can be generated by other methods known to those of skill in the art and tested by the high throughput methods herein. For purposes herein, a super-LEAD typically has activity with respect to the function of interest that differs from the improved activity of a LEAD by a desired amount, such as at least 5%, 10%, 20%, 30%, 40%, 50%, 60%, 70%, 80%, 90%, 100%, 150%, 200% or more from at least one of the LEAD mutants from which it is derived. As with LEADs, the change in the activity for super-LEADs is dependent upon the activity that is being “evolved.” The desired alteration, which can be either an increase or a reduction in activity, depends upon the function or property of interest.

As used herein, the phrase “altered loci” refers to the is-HIT amino acid positions in the LEADs or super-LEADs that are replaced with different replacing amino acids resulting in the desired altered phenotype or activity.

As used herein, an “exposed residue” presents more than 15% of its surface exposed to the solvent.

As used herein, the phrase “structural homology” refers to the degree of coincidence in space between two or more protein backbones. Protein backbones that adopt the same protein structure, fold and show similarity upon three-dimensional structural superposition in space can be considered structurally homologous. Structural homology is not based on sequence homology, but rather on three-dimensional homology. Two amino acids in two different proteins said to be homologous based on structural homology between those proteins do not necessarily need to be in sequence-based homologous regions. For example, protein backbones that have a root mean squared (RMS) deviation of less than 3.5, 3.0, 2.5, 2.0, 1.7 or 1.5 angstroms at a given space position or defined region between each other can be considered to be structurally homologous in that region and are referred to herein as having a “high coincidence” between their backbones. It is contemplated herein that substantially equivalent (e.g., “structurally related”) amino acid positions that are located on two or more different protein sequences that share a certain degree of structural homology have comparable functional tasks; also referred to herein as “structurally homologous loci.” These two amino acids then can be said to be “structurally similar” or “structurally related” with each other, even if their precise primary linear positions on the sequences of amino acids, when these sequences are aligned, do not match with each other. Amino acids that are “structurally related” can be far away from each other in the primary protein sequences, when these sequences are aligned following the rules of classical sequence homology. As used herein, a “structural homolog” is a protein that is generated by structural homology.

As used herein, “unmodified target protein,” “unmodified protein” or “unmodified cytokine,” “unmodified IFN-γ, ” “unmodified interferon-γ” or grammatical variations thereof refer to a starting protein that is selected for modification. The starting unmodified target protein includes a naturally-occurring, native wild-type form of a protein, or a recombinantly produced or synthetically produced polypeptide. Included among various forms of unmodified IFN-γ polypeptides that are contemplated for modification herein, are the 146 amino acid mature form of IFN-γ set forth in SEQ ID NO:1 and its precursor set forth in SEQ ID NO: 370, or any polymorphic form of IFN-γ thereof. Such a polymorphic form includes, but is not limited to, a mature IFN-γ polypeptide having a sequence of amino acids set forth in SEQ ID NO:2 and its precursor set forth in SEQ ID NO:371. Also included are IFN-γ polypeptides containing N- or C-terminal truncations as compared to the sequence of amino acids set forth in SEQ ID NO:1 or SEQ ID NO:2. For example, an unmodified IFN-γ polypeptide contemplated for modification herein includes those that are 124, 125, 126, 127, 128, 129, 130, 131, 132, 133, 134, 135, 136, 137, 138, 139, 140, 141, 142, 143, 144, 145, or 146 amino acid residues in length. Exemplary truncated forms of unmodified IFN-γ polypeptides are set forth in any of SEQ ID NOS: 372-379.

In addition, the starting unmodified target protein can have been altered or mutated, such that it differs from the native wild type isoform but is nonetheless referred to herein as a starting unmodified target protein relative to the subsequently modified proteins produced herein. Thus, existing proteins known in the art that have previously been modified to have a desired increase or decrease in a particular activity compared to an unmodified reference protein can be selected and used herein as the starting target protein for further modification. For example, a protein that has been modified from its native form by one or more single amino acid changes and possesses either an increase or decrease in a desired activity, such as resistance to proteolysis, can be used with the methods provided herein as the starting unmodified target protein for further modification of either the same or a different activity. For example, US Patent Publication No. US 2005/0249703 describes an S99T modification (corresponding to S102T based on the numbering of amino acids set forth in SEQ ID NO:1). Thus, an unmodified IFN-γ polypeptide includes a polypeptide containing a modification corresponding to S102T, that can be subsequently modified with any one or more amino acid modifications provided herein.

Existing proteins known in the art that previously have been modified to have a desired alteration, such as an increase or decrease, in a particular activity compared to an unmodified or reference protein can be selected and used as provided herein for identification of structurally homologous loci on other structurally homologous target proteins. For example, a protein that has been modified by one or more single amino acid changes and possesses either an increase or decrease in a desired activity, such as resistance to proteolysis, can be used with the methods provided herein to identify on structurally homologous target proteins, corresponding structurally homologous loci that can be replaced with suitable replacing amino acids and tested for either an increase or decrease in the desired activity.

As used herein, “variant,” “interferon-γ variant,” “modified IFN-γ polypeptides” and “modified IFN-γ proteins” refers to an IFN-γ polypeptide that has one or more mutations compared to an unmodified IFN-γ polypeptide. The one or more mutations can be one or amino acid replacements, insertions or deletions and any combination thereof. Typically, a modified IFN-γ polypeptide has one or more modifications in primary sequence compared to an unmodified interferon-γ. For example, a modified IFN-γ polypeptide provided herein can have 1, 2, 3, 2, 5, 6, 7, 8, 9, 10, 11, 12, 13, 14, 15, 16, 17, 18, 19, 20, or more mutations compared to an unmodified IFN-γ polypeptide. Modified IFN-γ polypeptides provided herein include the specified or recited modification, but can be produced as heterogeneous mixtures and/or can be produced with a variety of lengths, typically from 124 to 146 amino acids. Any length polypeptide is contemplated as long as the resulting polypeptide exhibits at least one IFN-γ activity associated with a longer form having at least 124 amino acids. Generally, for purposes herein, modification of an IFN-γ polypeptide is with respect to the monomer form. Typically, however, since IFN-γ is a dimer and active as a dimer, modifications are to the dimer as well (i.e. two IFN-γ polypeptides (the same or different) modified as described and non-covalently associated or linked by a linkage). For example, modification of an IFN-γ monomer can result in increased stability of the IFN-γ dimer due to stabilized interactions between monomers (i.e. interstability).

As used herein, an “IFN-γ polypeptide that has only a single mutation” refers to a modified IFN-γ polypeptide whose sequence, when aligned with the sequence of an unmodified IFN-γ polypeptide (of corresponding length), contains only one amino acid difference in amino acid sequence compared to the sequence of the unmodified IFN-γ polypeptide. For example, an IFN-γ polypeptide that has a single mutation includes a modified IFN-γ polypeptide that has an identical sequence to an unmodified IFN-γ, such as for example a native IFN-γ, except that the modified IFN-γ has an amino acid replacement at position D5 to an asparagine (N), where the aligned native sequence of IFN-γ contains aspartic acid (D) at position 5.

As used herein, an “IFN-γ polypeptide that has 2, 3, 4, 5, 6, 7, 8, 9, 10, 11, 12, 13, 14, 15, 16, 17, 18, 19, 20, or more mutations” refers to a modified IFN-γ polypeptide whose sequence, when aligned with a sequence of an unmodified IFN-γ polypeptide (of corresponding length), contains 2, 3, 4, 5, 6, 7, 8, 9, 10, 11, 12, 13, 14, 15, 16, 17, 18, 19, 20, or more amino acid differences in amino acid sequence compared to the sequence of the unmodified IFN-γ polypeptide. For example, an IFN-γ polypeptide that has 2 mutations includes a modified IFN-γ polypeptide that has an identical sequence to an unmodified IFN-γ polypeptide, such as for example a native IFN-γ polypeptide, except that the modified IFN-γ has an amino acid replacement at position D5 to an asparagine (N) and an amino acid replacement at position R143 to a histidine (H), where the aligned native sequence of IFN-γ contains aspartic acid (D) at position 5 and arginine (R) at position 143.

As used herein, “corresponding length” with reference to an unmodified polypeptide, means that the modified polypeptide is compared to an unmodified polypeptide of the same number of amino acids. As noted herein, IFN-γ polypeptides are heterogeneous in length. For determining the number of mutations, polypeptides of the same length are compared.

As used herein, a “single amino acid replacement” refers to the replacement of one amino acid by another amino acid. The replacement can be by a natural amino acid or non-natural amino acids. When one amino acid is replaced by another amino acid in a protein, the total number of amino acids in the protein is unchanged.

As used herein, the phrase “only one amino acid replacement occurs on each target protein” refers to the modification of a target protein, such that it differs from the unmodified form of the target protein by a single amino acid change. For example, in one embodiment, mutagenesis is performed by the replacement of a single amino acid residue at only one is-HIT target position on the protein backbone (e.g., “one-by-one” in addressable arrays), such that each individual mutant generated is the single product of each single mutagenesis reaction. The single amino acid replacement mutagenesis reactions are repeated for each of the replacing amino acids selected at each of the is-HIT target positions. Thus, a plurality of mutant protein molecules are produced, whereby each mutant protein contains a single amino acid replacement at only one of the is-HIT target positions.

As used herein, the phrase “pseudo-wild type,” in the context of single or multiple amino acid replacements, are those amino acids that, while different from the original (e.g., such as native) amino acid at a given amino acid position, can replace the native one at that position without introducing any measurable change in a particular protein activity. A population (library) of sets of nucleic acid molecules encoding a collection of mutant molecules is generated and phenotypically characterized such that proteins with sequences of amino acids different from the original amino acid, but that still elicit substantially the same level (i.e., at least 10%, 50%, 70%, 90%, 95%, 100%, depending upon the protein) and type of desired activity as the original protein are selected. A library, contains three, four, five, 10, 50, 100, 500, 1000, 103, 104 or more modified IFN-γ polypeptides.

As used herein, “in a position or positions corresponding to an amino acid position” of a protein, refers to amino acid positions that are determined to correspond to one another based on sequence and/or structural alignments with a specified reference protein. For example, in a position corresponding to an amino acid position of human interferon-γ set forth as SEQ ID NO: 1 can be determined empirically by aligning the sequence of amino acids set forth in SEQ ID NO: 1 with a particular interferon-γ of interest. Corresponding positions can be determined by such alignment by one of skill in the art using manual alignments or by using the numerous alignment programs available (for example, BLASTP). Corresponding positions also can be based on structural alignments, for example by using computer simulated alignments of protein structure. Recitation that amino acids of a polypeptide correspond to amino acids in a disclosed sequence refers to amino acids identified upon alignment of the polypeptide with the disclosed sequence to maximize identity or homology (where conserved amino acids are aligned) using a standard alignment algorithm, such as the GAP algorithm. As used herein, “at a position corresponding to” refers to a position of interest (i.e., base number or residue number) in a nucleic acid molecule or protein relative to the position in another reference nucleic acid molecule or protein. The position of interest to the position in another reference protein can be in, for example, a precursor protein, an allelic variant, a heterologous protein, an amino acid sequence from the same protein of another species, etc. For example, one of skill in the art recognizes that the referenced positions of SEQ ID NO: 1 differ by twenty amino acid residues when compared to SEQ ID NO: 370, which is a precursor form of IFN-γ containing a signal peptide at amino acids 1-20. Thus, the first amino acid residue of SEQ ID NO: 1 “corresponds to” the twenty first amino acid residue of SEQ ID NO: 370. Corresponding positions can be determined by comparing and aligning sequences to maximize the number of matching nucleotides or residues, for example, such that identity between the sequences is greater than 95%, preferably greater than 96%, more preferably greater than 97%, even more preferably greater than 98% and most preferably greater than 99%. The position of interest is then given the number assigned in the reference nucleic acid molecule.

By aligning the sequences of IFN-γ polypeptides, one skilled in the art can identify corresponding residues, using conserved and identical amino acid residues as guides. In other instances, corresponding regions can be identified. For example, L14 and Y17 of SEQ ID NO: 1 (mature IFN-γ) correspond to L34 and Y37 of SEQ ID NO: 370 (precursor IFN-γ with signal peptide). One skilled in the art also can employ conserved amino acid residues as guides to find corresponding amino acid residues between and among human and non-human sequences. For example, residues L14 and Y17 of SEQ ID NO: 1 correspond to L14 and Y17 of SEQ ID NO: 380 (chimpanzee) or L11 and Y14 of SEQ ID NO: 391 (gorilla) or L33 and Y36 of SEQ ID NO: 386 (mouse). In another example, one skilled in the art can determine that L34 and Y37 of SEQ ID NO: 370 correspond to L34 and Y37 of SEQ ID NOS: 381-385, 387-390 and 392-399.

As used herein, the terms “homology” and “identity” are used interchangeably, but homology for proteins can include conservative amino acid changes. In general to identify corresponding positions the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J. Applied Math 48:1073).

As use herein, “sequence identity” refers to the number of identical amino acids (or nucleotide bases) in a comparison between a test and a reference polypeptide or polynucleotide. Homologous polypeptides refer to a pre-determined number of identical or homologous amino acid residues. Homology includes conservative amino acid substitutions as well identical residues. Sequence identity can be determined by standard alignment algorithm programs used with default gap penalties established by each supplier. Homologous nucleic acid molecules refer to a pre-determined number of identical or homologous nucleotides. Homology includes substitutions that do not change the encoded amino acid (i.e., “silent substitutions”) as well identical residues. Substantially homologous nucleic acid molecules hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule. (For determination of homology of proteins, conservative amino acids can be aligned as well as identical amino acids; in this case, percentage of identity and percentage homology vary). Whether any two nucleic acid molecules have nucleotide sequences (or any two polypeptides have amino acid sequences) that are at least 80%, 85%, 90%, 95%, 96%, 97%, 98% or 99% “identical” can be determined using known computer algorithms such as the “FAST A” program, using for example, the default parameters as in Pearson et al. Proc. Natl. Acad. Sci. USA 85: 2444 (1988) (other programs include the GCG program package (Devereux, J., et al., Nucleic Acids Research 12(I): 387 (1984)), BLASTP, BLASTN, FASTA (Atschul, S. F., et al., J. Molec. Biol. 215:403 (1990); Guide to Huge Computers, Martin J. Bishop, ed., Academic Press, San Diego (1994), and Carillo et al. SIAM J Applied Math 48: 1073 (1988)). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. J. Mol. Biol. 48: 443 (1970), as revised by Smith and Waterman (Adv. Appl. Math. 2: 482 (1981)). Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds.,

Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide. For example, a test polypeptide can be defined as any polypeptide that is 90% or more identical to a reference polypeptide. In one non-limiting example, “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, “corresponding structurally-related” positions on two or more proteins, such as IFN-γ protein and other cytokines, refer to those amino acid positions determined based upon structural homology to maximize tri-dimensional overlapping between proteins.

As used herein, the phrase “sequence-related proteins” refers to proteins that have at least 50%, at least 60%, at least 70%, at least 80%, at least 90%, or at least 95% amino acid sequence identity or homology with each other.

As used herein, families of non-related proteins or “sequence-non-related proteins” refer to proteins having less than 50%, less than 40%, less than 30%, less than 20% amino acid identity or homology with each other.

As used herein, it also is understood that the terms “substantially identical” or “similar” varies with the context as understood by those skilled in the relevant art.

As used herein, “a naked polypeptide chain” refers to a polypeptide that is not post-translationally modified or otherwise chemically modified, but contains only covalently linked amino acids.

As used herein, a polypeptide complex includes polypeptides produced by chemical modification or post-translational modification. Such modifications include, but are not limited to, pegylation, albumination, glycosylation, farnysylation, phosphorylation and other polypeptide modifications known in the art.

As used herein, “output signal” refers to parameters that can be followed over time and, optionally, quantified. For example, when a recombinant protein is introduced into a cell, the cell containing the recombinant protein undergoes a number of changes. Any such change that can be monitored and used to assess the transformation or transfection is an output signal, and the cell is referred to as a reporter cell; the encoding nucleic acid is referred to as a reporter gene; and the construct that includes the encoding nucleic acid is a reporter construct. Output signals include, but are not limited to, enzyme activity, fluorescence, luminescence, amount of product produced and other such signals. Output signals include expression of a gene or gene product, including heterologous genes (transgenes) inserted into the plasmid virus. Output signals are a function of time (“t”) and are related to the amount of protein used in the composition. For higher concentrations of protein, the output signal can be higher or lower. For any particular concentration, the output signal increases as a function of time until a plateau is reached. Output signals also can measure the interaction between cells, expressing heterologous genes and biological agents.

As used herein, the Hill equation is a mathematical model that relates the concentration of a drug (i.e., test compound or substance) to the response measured

$$y = \frac{{{y}_{max} [ D ]}^{x}}{{[ D ]}^{n} + {[ {D}_{50} ]}^{n}}$$

where y is the variable measured (e.g., such as a response signal) yₘₐₓ is the maximal response achievable, [D] is the molar concentration of a drug, [D₅₀] is the concentration that produces a 50% maximal response to the drug, n is the slope parameter, which is 1 if the drug binds to a single site and with no cooperativity between or among sites. A Hill plot is log₁₀ of the ratio of ligand-occupied receptor to free receptor vs log [D] (M). The slope is n, where a slope of greater than 1 indicates cooperativity among binding sites and a slope of less than 1 can indicate heterogeneity of binding. This equation has been employed in methods for assessing interactions in complex biological systems (see, published International PCT application No. WO 01/44809 based on PCT No. PCT/FR00/03503).

As used herein, in the Hill-based analysis (published International PCT application No. WO 01/44809 based on PCT No. PCT/FR00/03503), the parameters, π, κ, τ, ε, η, θ, are as follows:
c is the potency of the biological agent acting on the assay (cell-based) system;
κ is the constant of resistance of the assay system to elicit a response to a biological agent;
ε is the global efficiency of the process or reaction triggered by the biological agent on the assay system;
τ is the apparent titer of the biological agent;
θ is the absolute titer of the biological agent; and
η is the heterogeneity of the biological process or reaction.

In particular, as used herein, the parameters π (potency) or κ (constant of resistance) are used, respectively, to assess the potency of a test agent to produce a response in an assay system and the resistance of the assay system to respond to the agent.

As used herein, ε (efficiency) is the slope at the inflexion point of the Hill curve (or, in general, of any other sigmoidal or linear approximation), to assess the efficiency of the global reaction (the biological agent and the assay system taken together) to elicit the biological or pharmacological response.

As used herein, τ (apparent titer) is used to measure the limiting dilution or the apparent titer of the biological agent.

As used herein, θ (absolute titer) is used to measure the absolute limiting dilution or titer of the biological agent.

As used herein, η (heterogeneity) measures the existence of discontinuous phases along the global reaction, which is reflected by an abrupt change in the value of the Hill coefficient or in the constant of resistance.

As used herein, a population of sets of nucleic acid molecules encoding a collection (library) of mutants refers to a collection of plasmids or other vehicles that carry (encode) the gene variants. Thus, individual plasmids or other individual vehicles carry individual gene variants. Each element (member) of the collection is physically separated from the others in an appropriate addressable array and has been generated as the single product of an independent mutagenesis reaction. When a collection (library) of such proteins is contemplated, it will be so-stated.

As used herein, “IFN-γ-mediated disease or disorder” refers to any disease or disorder in which treatment with IFN-γ ameliorates any symptom or manifestation of the disease or disorder. Exemplary IFN-γ-mediated diseases and disorders include, but are not limited to, chronic granulomatous disease (CGD); idiopathic pulmonary fibrosis (IPF); proliferative disorders such as bone disorders (e.g., malignant osteopetrosis), atherosclerosis, restenosis and cancers such as ovarian cancer; viral infections such as hepatitis C (HCV) and viral conditions such as hepatitis C virus and human immunodeficiency virus; bacterial infections such as tuberculosis, fungal infections such as aspergillosis; and hyper-IgE states such as allergic dermatitis, asthma and other allergic responses.

As used herein, cancer refers to the development and growth of abnormal cells in an uncontrolled manner as is commonly understood by those of skill in the the art. Cancers include solid tumors and blood born cancers, such as leukemias. Cancers tend to invade surrounding tissues, and spread to distant sites of the body via the blood stream and lymphatic system. Cancer includes any of various malignant neoplasms characterized by the proliferation of anaplastic cells that tend to invade surrounding tissue and metastasize to new body sites. Cancer includes lung, prostate, bladder, breast, cervical, kidney and ovarian cancers and also lymphomas and leukemias.

As used herein, a tumor refers to an abnormal growth of tissue resulting from uncontrolled, progressive multiplication of cells with no physiological function or to a neoplasm.

As used herein, cancer cells include malignant neoplastic, anaplastic, metastatis, hyperplastic, dysplastic, neoplastic, malignant tumor (solid or blood-borne) cells that display abnormal growth in the body in an uncontrolled manner.

As used herein, neoplasm refers to new and abnormal growth of tissue, which can be cancerous, such as a malignant tumor.

As used herein, neoplastic disease, means a disease brought about by the existence of a neoplasm in the body.

As used herein, metastasis refers to the migration of cancerous cells to other parts of the body.

As used herein, hyperplasia refers to an abnormal increase in the number of cells in an organ or a tissue with consequent enlargement. As used herein, neoplasm and dysplasia refer to abnormal growth of tissues, organs or cells. As used herein, malignant means an cancerous or tending to metastasize. As used herein, anaplastic means cells that have become less differentiated.

As used herein, leukemia refers to a cancer of the blood cells. Any of various acute or chronic neoplastic diseases of the bone marrow in which unrestrained proliferation of white blood cells occurs, usually accompanied by anemia, impaired blood clotting, and enlargement of the lymph nodes, liver and spleen. Leukemia occurs when bone marrow cells multiply abnormally cased by mutations in the DNA of stem cells. Bone marrow stem cells, as used herein, refer to undifferentiated stem cells that differentiate into red blood cells and white blood cells. Leukemia is characterized by an excessive production of abnormal white blood cells, overcrowding the bone marrow and spilling into peripheral blood. Various types of leukemias spread to lymph nodes, spleen, liver, the central nervous system and other organs and tissues.

As used herein, lymphoma refers to a malignant tumor that arises in the lymph nodes or other lymphoid tissue.

As used herein, malignant osteopetrosis (OP) refers to an inherited condition characterized by an osteoclast defect. The disease is characterized by bone overgrowth and deficient phagocyte oxidative metabolism. Osteoclasts are cells of monocyte/macrophage origin that erode bone matrix and regulation of their differentiation is central to the understanding of the pathogenesis and treatment of bone diseases such as osteopetrosis. Bone-resorbing osteoclasts and bone-forming osteoblasts are essential to maintaining a balance between bone resorption and formation.

As used herein, atherosclerosis refers to a form of arteriosclerosis characterized by the deposition of atheromatous plaques containing cholesterol and lipids on the innermost layer of the walls of large and medium-sized arteries, inflammation, and fibrous tissue formation. As used herein, hyperplasia refers to an abnormal increase in the number of cells in an organ or a tissue with consequent enlargement. As used herein, restenosis refers to a recurrence of abnormal narrowing in a blood vessel or other tubular organ or structure after corrective surgery on a heart valve.

As used herein, Marek's disease virus (MDV) refers to a herpesvirus-induced lymphoproliferative disease of chickens. Infections in chickens start with a lytic infection in B cells followed by latent infection in T cells. T cell lymphomas develop in susceptible birds.

As used herein, mycobacterium refers to the genus of actinobacteria given its own family (i.e., Mycobacteriaceae). Mycobacterium refers to any of various slender, rod-shaped, aerobic bacteria of the genus Mycobacterium, which includes bacteria that cause tuberculosis and leprosy. The mycobacterial cell wall makes a substantial contribution to the hardiness of this genus, thus mycobacterial infections are notoriously difficult to treat, which naturally leads to antibiotic resistance. Mycobacteria can be classified into several major groups for purpose of diagnosis and treatment: (1) M. tuberculosis complex which can cause tuberculosis: M. tuberculosis, M. bovis, M. africanum, and M. micoti, (2) M. leprae which causes Hansen's disease or leprosy, and (3) Non-tuberculous mycobacteria (NTM) are all the other mycobacteria which can cause pulmonary disease resembling tuberculosis, lymphadenitis, skin disease, or disseminated disease.

As used herein, tuberculosis (TB) refers to an infectious disease of humans and animals caused by Mycobacterium tuberculosis and is characterized by the formation of tubercles on the lungs and other tissues of the body, which tubercles often develop long after the initial infection. Antibiotic regimens require at least 6 months duration because latent or stationary phase organisms are difficult to kill and, generally, do not fully clear the bacterium from the infected subject. Tuberculosis is one of the first secondary-type infections to be activated in immunocompromised HIV patients and cases of drug-resistant strains are increasing (Rook et al. Eur. Respir. J. 17: 537-557 (2001)). TB most commonly affects the lungs (pulmonary tuberculosis) but also can involve most any organ of the body. M. tuberculosis infections are characterized by ineffective Th2 responses which increase susceptibility of infected patients and undermine the efficacy of Th1 responses needed to eradicate infection. The important role of Th1 responses (e.g., IFN-γ) in resistance to M. tuberculosis comes from children defective in IFN-γ production and from animal (mouse) models. Inhibition or killing of M. tuberculosis is induced in murine macrophages (likely due to reactive oxygen and nitrogen intermediates) after exposure to IFN-γ.

As used herein, leprosy, also called Hansen's disease, refers to a chronic, mildly contagious granulomatous disease of tropical and subtropical regions, caused by the bacillus Mycobacterium leprae.

As used herein, non-tuberculous mycobacteria (NTM) refers to mycobacteria other than M. leprae that are not included in the M. tuberculosis complex. NTMs are distinguished by groups based on production of pigment, speed of growth, and analysis of species-specific mycolic acids. As a group, NTM are ubiquitous in the environment. Most species are either not pathogenic for humans or are rarely associated with disease. Some species, such as M. avium, are opportunistic pathogens that are more frequently associated with disease, especially in immunocompromised persons. The most frequent bacterial infections in patients infected with HIV and suffering from AIDS are non-tubercolous mycobacterial infections. Incidences of infection are increasing as survival of patients being treated with anti-retroviral drugs is prolonged (Payen et al. Rev. Mal. Respir. 14(Suppl 5): 142-151 (1997)). Mycobacterium avium complex (MAC) infects most, if not all, HIV-positive patients.

As used herein, aspergillosis refers to an infection or an allergic response caused by fungi of the genus Aspergillus. Although it may play a role in allergy, it is best known for causing serious pulmonary infections in immunocompromised patients. The incidence of invasive aspergillosis is increasing parallel to the intensity of immunosuppressive and myelosuppressive anti-cancer treatments and is a major cause for morbidity and mortality in neutropenic patients. Aspergillosis causes illness in three ways: (1) as an allergic reaction in people with asthma (pulmonary aspergillosis—allergic bronchopulmonary type); (2) as a colonization and growth in an old healed lung cavity from previous disease (such as tuberculosis or lung abscess) where it produces a fungus ball called aspergilloma; or (3) as an invasive infection with pneumonia that is spread to other parts of the body by the bloodstream (pulmonary aspergillosis—invasive type). The invasive infection can affect the eye, causing blindness, and any other organ of the body, but especially the heart, lungs, brain, and kidneys. The third form occurs almost exclusively in people who are immunosuppressed because of cancer, AIDS, leukemia, organ transplants, high doses of corticosteroid drugs, chemotherapy, or other diseases that reduce the number of normal white blood cells. Aspergillus spores release factors that can suppress the synthesis of pro-inflammatory Th1 cytokines, and Th2 reactivity has been shown to lead to disease progression.

As used herein, candidiasis refers to an infection with a fungus of the genus Candida, especially C. albicans, that usually occurs in the skin and mucous membranes of the mouth, respiratory tract, or vagina but may invade the bloodstream, especially in immuno-compromised individuals. In immunocompetent people, candidiasis can usually only be found in exposed and moist parts of the body, such as the oral cavity (oral thrush), the vagina (vaginal candidiasis or thrush), the penis (balanitis) or folds of skin in the diaper area (diaper rash), and its growth is usually kept in check by certain bacteria that also live in these areas. When the balance of these organisms is disturbed by antibiotic treatment, by hormonal imbalances, or by a weakening of the body's resistance to disease (as occurs in AIDS or chemotherapy), the fungus proliferates. Host responses to fungi are a result of interactions between the innate and adaptive immune systems. Neutrophils and monocytes are involved in the non-specific clearance of yeasts such as Candida albicans and Th1 responses are protective via release or administration of IFN-γ. In contrast, Th2 responses (e.g., IL-4 and IL-10) exacerbate the disease and pathology.

As used herein, Pneumocystis jiroveci (formerly known as Pneumocystis carinii) refers to a fungus that is the causative agent of Pneumocystis carinii pneumonia (PCP). It is relatively rare in normal, immunocompetent people, and most commonly affects those with weakened immune systems such as children and the elderly (but is especially common in patients with AIDS) and frequently causes mortality. In immunocompromised AIDS patients having a low CD4⁺ count (i.e., below 200/μl), prophylaxis may be necessary because despite the development of antiretroviral therapy. PCP in AIDS patients is due, in part, to impaired local release of IFN-γ from lung lymphocytes and subsequent failure to activate macrophages.

As used herein, coccidioidomycosis, also called valley fever, refers to an infectious respiratory disease of humans and other animals caused by inhaling dust containing spores of the fungus Coccidioides immitis and is characterized by fever and various respiratory symptoms. Although some cases of coccidioidomycoses are asymptomatic, symptomatic infection usually presents as an influenza-like illness with fever, cough, headaches, rash, and myalgias. Some patients fail to recover and develop chronic pulmonary infection or widespread disseminated infection affecting meninges, soft tissues, joints, and bone. From the respiratory tract, it can spread to the skin, bones, and central nervous system. Severe pulmonary disease may develop in HIV-infected persons.

As used herein, histoplasmosis refers to a disease caused by the inhalation of spores of the fungus Histoplasma capsulatum. H. capsulatum grows in soil and material contaminated with bat or bird droppings. Spores become airborne when contaminated soil is disturbed. Disease symptoms vary greatly, but the disease primarily affects the lungs. Most often, subjects are asymptomatic, but occasionally subjects produce acute pneumonia or an influenza-like illness that spreads to other organs and systems in the body (i.e., disseminated histoplasmosis), and it can be fatal if untreated.

As used herein, Cryptococcus neoformans refers to a species of fungus that can live in both plants and animals, and most commonly enters the body through the lungs. In humans, it does not affect health subjects, but it can cause serious illness (such as meningitis or brain abscess) or death in immunocompromised subjects. Infection usually does not appear until a person's CD4+ T cell counts have dropped below 100/μl. C. neoformans most commonly affects the brain, causing the condition called meningitis. Meningitis is an infection and swelling of the lining of the brain and spinal cord and is a life-threatening infection. Cryptococcus also can cause infections of the lungs, skin and prostate gland. In immunocompetent patients, endogenous IFN-γ protects against C. neoformans infection by inducing cellular inflammatory responses, potentiating clearance of microorganism from the lungs and preventing its dissemination into the central nervous system.

As used herein, Paracoccidioides brasiliensis refers to a fungus that causes an infection characterized by a chronic inflammatory granulomatous reaction. The infection is acquired by inhalation of airborne propagules produced by the fungal mycelium form, which then change into the pathogenic yeast when at core body temperature. The absence of IFN-γ leads to incipient granulomas which are unable to control spread of the fungus (Suoto et al. Am. J. Pathology 156(5): 1811-1820 (2000); Suoto et al. Am. J. Pathology 163(2): 583-590 (2003); Calvi et al. Microbes Infect. 5(2): 107-113 (2003)).

As used herein, protozoan parasites cause infections that are characterized as intracellular infections that take place primarily in macrophages. IFN-γ provides one of the major signals for activation of macrophages, which are required for killing parasitic protozoans. As used herein, toxoplasmosis refers a parasitic disease caused by the parasite Toxoplasma gondii. The parasite rarely causes any symptoms in otherwise healthy adults but, in people with a weakened immune system such as people infected with HIV, the parasite can cause encephalitis (infection of the brain), neurologic diseases and can affect the heart, liver, and eyes (chorioretinitis).

As used herein, chronic granulomatous disease (CGD), also called congenital dysphagocytosis, refers to an inherited disorder characterized by deficient phagocyte oxidative mechanisms and a congenital defect in the ability of polymorphonuclear leukocytes to kill phagocytized bacteria, resulting in increased susceptibility to severe bacterial and fungal infections. CGD is a rare disorder that includes a lack of production of superoxide and hydrogen peroxide in phagocytes. Neutrophils require a set of enzymes to produce reactive oxygen species to destroy bacteria after their phagocytosis. Together these enzymes are termed “phagocyte NADPH oxidase” (phox). Defects in one of these enzymes can cause CGD of varying severity dependent on the defect. Four genes have been implicated in CGD (p is the weight of the protein in kDa; the g means glycoprotein): CYBB, coding the gp91-phox subunit (X-linked, accounts for ⅔ of the cases); CYBA, coding p22-phox; NCF-1, coding p47-phox; and NCF-2, coding p67-phox.

As used herein, fibrosis refers to a pathologic process that includes scarring (fibrosis) and over-production of the extracellular matrix by the connective tissue, as a response to tissue damage. Pulmonary fibrosis occurs throughout the lungs and leads to progressive deterioration and destruction of the tissue. In some patients, chronic pulmonary inflammation and fibrosis develop without identifiable cause, a condition called idiopathic pulmonary fibrosis (IPF).

As used herein, “atopic” refers to diseases that are hereditary, tend to run in families, and often occur together. Atopic diseases include, for example, asthma, hay fever, and atopic dermatitis, each of which is associated with elevated IL-4 and IgE levels. The use of IFN-γ in diseases or disorders associated with extreme levels of serum IgE derives from the ability of IFN-γ to regulate production of IgE by B cells. IFN-γ acts directly on differentiated B cells to induce production of antibody isoforms other than IgG4 or IgE. IFN-γ also is a potent inhibitor if IL-4 production.

As used herein, atopic dermatitis refers to a chronic inflammatory disease that affects the skin which becomes extremely itchy and inflamed, causing redness, swelling, cracking, weeping, crusting, and scaling. Atopic dermatitis most often affects infants and young children, but it can continue into adulthood or first show up later in life. In most cases, there are periods of time when the disease is worse (i.e., exacerbations or flares), which are followed by periods when the skin improves or clears up entirely (i.e., remissions). Many children with atopic dermatitis enter into a permanent remission of the disease when they get older, although their skin often remains dry and easily irritated. Environmental factors can activate symptoms of atopic dermatitis at any time in the lives of individuals who have inherited the atopic disease trait. Atopic dermatitis is the most common of the many types of eczema and is a chronic skin disease characterized by itchy, inflamed skin.

As used herein, asthma refers to a chronic inflammatory respiratory disease, often arising from allergies, that is characterized by sudden recurring attacks of labored breathing, chest constriction, coughing and periodic attacks of wheezing. A cough producing sticky mucus is symptomatic and the symptoms often appear to be caused by the body's reaction to a trigger such as an allergen (commonly pollen, house dust, animal dander: see allergy), certain drugs, an irritant (such as cigarette smoke or workplace chemicals), exercise, or emotional stress. These triggers can cause the asthmatic's lungs to release chemicals that create inflammation of the bronchial lining, constriction, and bronchial spasms. If the effect on the bronchi becomes severe enough to impede exhalation, carbon dioxide can build up in the lungs and lead to unconsciousness and death. The respiratory tract is commonly infected by a range of viruses with overlapping pathologies and the majority of episodic exacerbations of asthma are associated with viral infection (e.g., rhinovirus infections).

As used herein, allergic diseases refer to a disease that is a result of an immune reaction to foreign substances (i.e., allergens) that are typically harmless, but which, in some subjects, cause an adverse reaction. Subject with allergies, commonly known as hay fever (allergic rhinitis), have an abnormally high sensitivity to certain substances, such as pollens, foods, or microorganisms. Exemplary allergens include, but are not limited to, pollens, dust mite, molds, danders, and certain foods. Subjects prone to allergies are said to be allergic or atopic. Although allergies can develop at any age, the risk of developing allergies is genetic. Stimulation of allergen-specific T cells by allergen-derived peptides, presented by antigen presenting cells on class II MHC molecules results in differentiation of CD4+ T cells into Th2 cytokine producing cells which produce IL-4, IL-13 and IL-5 which regulate the allergic response (Wills-Karp et al. Nature Reviews Immunology 1: 69-75 (2001)). Allergens cause the production of immunoglobulin E (IgE) in abnormally quantities. During the sensitization period in allergy, IgE is overproduced and coats cells such as mast cells and basophils that contain chemicals such as histamine. These chemicals, in turn, cause inflammation and typical allergic symptoms. Hay fever (allergic rhinitis) is the most common of the allergic diseases and refers to seasonal nasal symptoms that are due to outdoor allergens such as pollens or ragweed and/or indoor allergens, such as dust mites or molds.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of a modified interferon and compositions provided herein.

As used herein, “therapeutically effective amount” or “therapeutically effective dose” refers to an agent, compound, material, or composition containing a compound that is at least sufficient to produce a therapeutic effect.

As used herein, “patient” or “subject” to be treated includes humans and human or non-human animals. Mammals include, primates, such as humans, chimpanzees, a gorillas and monkeys; a domesticated animals, such as dogs, horses, cats, pigs, goats, cows; and rodents such as mice, rats, hamsters and gerbils.

As used herein, a “reporter cell” is the cell that undergoes the change in response to a condition. For example, in response to exposure to a protein or a virus or to a change it its external or internal environment, the reporter cell “reports” (i.e., displays or exhibits the change).

As used herein, “reporter” or “reporter moiety” refers to any moiety that allows for the detection of a molecule of interest, such as a protein expressed by a cell. Reporter moieties include, but are not limited to, fluorescent proteins (e.g., red, blue and green fluorescent proteins), LacZ and other detectable proteins and gene products. For expression in cells, nucleic acids encoding the reporter moiety can be expressed as a fusion protein with a protein of interest or under to the control of a promoter of interest.

As used herein, phenotype refers to the physical, physiological or other manifestation of a genotype (a sequence of a gene). In methods herein, phenotypes that result from alteration of a genotype are assessed.

As used herein, peripheral blood monocytes (PBMC) include autologous and allogeneic cells.

As used herein, culture medium is any medium suitable for supporting the viability, growth, and/or differentiation of mammalian cells ex vivo. Any such medium known to those of skill in the art. Examples of culture medium include, but are not limited to, X-Vivo15 (BioWhittaker), RPMI 1640, DMEM, Ham's F12, McCoys 5A and Medium 199. The medium can be supplemented with additional ingredients including serum, serum proteins, growth suppressing, and growth promoting substances, such as mitogenic monoclonal antibodies and selective agents for selecting genetically engineered or modified cells.

As used herein, a resting T-cell means a T-cell that is not dividing or producing cytokines. Resting T-cells are small (approximately 6-8 microns) in size compared to activated T-cells (approximately 12-15 microns).

As used herein, a primed T-cell is a resting T-cell that has been previously activated at least once and has been removed from the activation stimulus for at least 48 hours. Primed T-cells usually have a memory phenotype.

As used herein, an activated T-cell is a T-cell that has received at least two mitogenic signals. As a result of activation, a T-cell will flux calcium which results in a cascade of events leading to division and cytokine production. Activated T-cells can be identified phenotypically, for example, by virtue of their expression of CD25. Cells that express the IL-2 receptor (CD25) are referred to herein as “activated”. A pure or highly pure population of activated cells typically express greater than 85% positive for CD25.

As used herein, immune cells are the subset of blood cells known as white blood cells, which include mononuclear cells such as lymphocytes, monocytes, macrophages and granulocytes.

As used herein, T-cells are lymphocytes that express the CD3 antigen.

As used herein, helper cells are CD4+ lymphocytes.

As used herein, regulatory cells are a subset of T-cells, most commonly CD4+ T-cells, that are capable of enhancing or suppressing an immune response. Regulatory immune cells regulate an immune response primarily by virtue of their cytokine secretion profile. Some regulatory immune cells also can act to enhance or suppress an immune response by virtue of antigens expressed on their cell surface and mediate their effects through cell-to-cell contact. Th1 and Th2 cells are examples of regulatory cells.

As used herein, effector cells are immune cells that primarily act to eliminate tumors or pathogens through direct interaction, such as phagocytosis, perforin and/or granulozyme secretion, induction of apoptosis, etc. Effector cells generally require the support of regulatory cells to function and also act as the mediators of delayed type hypersensitivity reactions and cytotoxic functions. Examples of effector cells are B lymphocytes, macrophages, cytotoxic lymphocytes, LAK cells, NK cells and neutrophils.

As used herein, T cells that produce IFN-γ, and not IL-4 upon stimulation are referred to as Th1 cells. Cells that produce IL-4, and not IFN-γ, are referred to as Th2 cells. A method for identifying Th1 cells in a population of cells is to stain the cells internally for IFN-γ. Th2 cells are commonly identified by internal staining for IL-4. In normal (i.e., subjects not exhibiting overt disease) individuals, generally only about 12-16% of the CD4+ cells stain positive for internal IFN-γ after activation; less than 1% stain positive for IFN-γ prior to activation. It is rare for a T-cell population to stain greater than 35% IFN-γ positive.

As used herein, immune balance refers to the normal ratios, and absolute numbers, of various immune cells and their cytokines that are associated with a disease free state. Restoration of immune balance refers to restoration to a condition in which treatment of the disease or disorder is effected whereby the ratios of regulatory immune cell types or their cytokines and numbers or amounts thereof are within normal range or close enough thereto so that symptoms of the treated disease or disorder are ameliorated.

As used herein, a disease characterized by a lack of Th1 cytokine activity refers to a state, disease or condition where the algebraic sum of cytokines in a specific microenvironment in the body or in a lesion(s) or systemically is less than the amount of Th1 cytokines present normally found in such microenvironment or systemically (i.e., in the subject or another such subject prior to onset of such state, disease or condition). The cytokines to assess include IFN-γ, IL-2, and TNF-α. The precise amounts and cytokines to assess depend upon the particular state, disease or condition.

As used herein, diseases characterized by a Th2-dominated immune response are characterized by either a suppressed cellular immune response or excessive humoral response.

As used herein, a disease characterized by an excess of Th2 cytokine activity refers to a state, disease or condition where the algebraic sum of cytokines in a specific microenvironment in the body or in a lesion(s) or systemically is predominantly of the Th2 type, dominated by IL-4 and/or IL-10 and/or TGF-α. Diseases, states or conditions that exhibit enhanced Th2 responses include infectious diseases such as, but are not limited to, virus infection, hyper-IgE states, leprosy, toxoplasmosis infection and AIDS. Imbalance in favor of Th2 cells occurs diseases that exhibit suppressed cellular immunity.

As used herein, immunotherapy refers to a treatment modality that seeks to harness the power of the human immune system to treat disease. Immunotherapy seeks to either enhance the immune response in diseases characterized by immunosuppression or suppress the immune response in subjects with diseases characterized by an overactive immune response.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide comprises two or more amino acids. For purposes herein, amino acids include the twenty naturally-occurring amino acids, non-natural amino acids and amino acid analogs (i.e., amino acids wherein the α-carbon has a side chain).

As used herein, the abbreviations for any protective groups, amino acids and other compounds are, unless indicated otherwise, in accord with their common usage, recognized abbreviations, or the IUPAC-IUB Commission on Biochemical Nomenclature (1972) Biochem. 11: 1726). Each naturally occurring L-amino acid is identified by the standard three letter code (or single letter code) or the standard three letter code (or single letter code) with the prefix “L-;” the prefix “D-” indicates that the stereoisomeric form of the amino acid is D.

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. “NH₂” refers to the free amino group present at the amino terminus of a polypeptide. “COOH” refers to the free carboxy group present at the carboxyl terminus of a polypeptide. In keeping with standard polypeptide nomenclature described in J. Biol. Chem., 243: 3552-3559 (1969), and adopted 37 C.F.R. §§ 1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1) and modified and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-isostereomers of amino acids. Exemplary non-natural amino acids are described herein and are known to those of skill in the art.

As used herein, nucleic acids include DNA, RNA and analogs thereof, including protein nucleic acids (PNA) and mixtures thereof. Nucleic acids can be single- or double-stranded. When referring to probes or primers (optionally labeled with a detectable label, e.g., a fluorescent or a radiolabel), single-stranded molecules are contemplated. Such molecules are typically of a length such that they are statistically unique of low copy number (typically less than 5, generally less than 3) for probing or priming a library. Generally a probe or primer contains at least 14, 16 or 30 contiguous of sequence complementary to, or identical to, a gene of interest. Probes and primers can be 10, 14, 16, 20, 30, 50, 100 or more nucleic acid bases long.

As used herein, heterologous or foreign nucleic acid, such as DNA and RNA, are used interchangeably and refer to DNA or RNA that does not occur naturally as part of the genome in which it occurs or is found at a locus or loci in a genome that differs from that in which it occurs in nature. Heterologous nucleic acid includes nucleic acid not endogenous to the cell into which it is introduced, but that has been obtained from another cell or prepared synthetically. Generally, although not necessarily, such nucleic acid encodes RNA and proteins that are not normally produced by the cell in which it is expressed. Heterologous DNA herein encompasses any DNA or RNA that one of skill in the art recognizes or considers as heterologous or foreign to the cell or locus in or at which it is expressed. Heterologous DNA and RNA also can encode RNA or proteins that mediate or alter expression of endogenous DNA by affecting transcription, translation, or other regulatable biochemical processes. Examples of heterologous nucleic acid include, but are not limited to, nucleic acid that encodes traceable marker proteins (e.g., a protein that confers drug resistance), nucleic acid that encodes therapeutically effective substances (e.g., anti-cancer agents), enzymes and hormones, and DNA that encodes other types of proteins (e.g., antibodies). Hence, herein heterologous DNA or foreign DNA, includes a DNA molecule not present in the exact orientation and position as the counterpart DNA molecule found in the genome. It also can refer to a DNA molecule from another organism or species (i.e., exogenous).

As used herein, “isolated with reference to a nucleic acid molecule or polypeptide or other biomolecule” means that the nucleic acid or polypeptide has separated from the genetic environment from which the polypeptide or nucleic acid were obtained. It also can mean altered from the natural state. For example, a polynucleotide or a polypeptide naturally present in a living animal is not “isolated,” but the same polynucleotide or polypeptide separated from the coexisting materials of its natural state is “isolated,” as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been partially or substantially purified from a recombinant host cell or from a native source. For example, a recombinantly produced version of a compound can be substantially purified by the one-step method described in Smith et al., Gene, 67:31-40 (1988). The terms isolated and purified can be used interchangeably.

Thus, by “isolated” it is meant that the nucleic acid is free of coding sequences of those genes that, in the naturally-occurring genome of the organism (if any), immediately flank the gene encoding the nucleic acid of interest. Isolated DNA can be single-stranded or double-stranded, and can be genomic DNA, cDNA, recombinant hybrid DNA or synthetic DNA. It can be identical to a starting DNA sequence or can differ from such sequence by the deletion, addition, or substitution of one or more nucleotides.

“Isolated” or “purified” preparations made from biological cells or hosts mean cell extracts containing the indicated DNA or protein including a crude extract of the DNA or protein of interest. For example, in the case of a protein, a purified preparation can be obtained following an individual technique or a series of preparative or biochemical techniques, and the DNA or protein of interest can be present at various degrees of purity in these preparations. The procedures can include, but are not limited to, ammonium sulfate fractionation, gel filtration, ion exchange chromatography, affinity chromatography, density gradient centrifugation and electrophoresis.

A preparation of DNA or protein that is “substantially pure” or “isolated” should be understood to mean a preparation free from naturally-occurring materials with which such DNA or protein is normally associated in nature. “Essentially pure” should be understood to mean a highly purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, “a targeting agent” refers to any molecule that can bind another target-molecule, such as an antibody, receptor or ligand.

As used herein, “receptor” refers to a biologically active molecule that specifically binds to (or with) other molecules. The term “receptor protein” can be used to more specifically indicate the proteinaceous nature of a specific receptor.

As used herein, “recombinant” refers to any progeny formed as the result of genetic engineering.

As used herein, a “promoter region” refers to the portion of DNA of a gene that controls transcription of the DNA to which it is operatively linked. The promoter region includes specific sequences of DNA sufficient for RNA polymerase recognition, binding and transcription initiation. This portion of the promoter region is referred to as the “promoter”. In addition, the promoter region includes sequences that modulate this recognition, binding and transcription initiation activity of the RNA polymerase. Promoters, depending upon the nature of the regulation, can be constitutive or regulated by cis acting or trans acting factors.

As used herein, the phrase “operatively linked” generally means the sequences or segments have been covalently joined into one piece of DNA, whether in single- or double-stranded form, whereby control or regulatory sequences on one segment control or permit expression or replication or other such control of other segments. The two segments are not necessarily contiguous. For gene expression, a DNA sequence and a regulatory sequence(s) are connected in such a way to control or permit gene expression when the appropriate molecular, e.g., transcriptional activator proteins, are bound to the regulatory sequence(s).

As used herein, “production by recombinant means by using recombinant DNA methods” means the use of the well-known methods of molecular biology for expressing proteins encoded by cloned DNA, including cloning expression of genes and methods, such as gene shuffling and phage display with screening for desired specificities.

As used herein, a splice variant refers to a variant produced by differential processing of a primary transcript of genomic DNA that results in more than one type of mRNA.

As used herein, a composition refers to any mixture of two or more products or compounds (e.g., agents, modulators, regulators, etc.). It can be a solution, a suspension, liquid, powder, a paste, aqueous, non-aqueous formulations or any combination thereof.

As used herein, a combination refers to any association between two or more items.

As used herein, an “article of manufacture” is a product that is made and sold. As used throughout this application, the term is intended to encompass pharmaceutical compositions of modified interferon-γ polypeptides and/or nucleic acids as described herein contained in articles of packaging.

As used herein, a “kit” refers to a combination of a modified interferon-γ polypeptides or nucleic acid molecules as described herein provided in pharmaceutical compositions and another item for a purpose including, but not limited to, administration, diagnosis, and assessment of an activity or property of the polypeptides described herein. Kits, optionally, include instructions for use.

As used herein, “substantially identical to a product” means sufficiently similar so that the property of interest is sufficiently unchanged so that the substantially identical product can be used in place of the product.

As used herein, the term “vector” refers to a nucleic acid molecule capable of transporting another nucleic acid to which it has been linked. One type of exemplary vector is an episome, i.e., a nucleic acid capable of extra-chromosomal replication. Exemplary vectors are those capable of autonomous replication and/or expression of nucleic acids to which they are linked; such vectors typically include origins of replication. Vectors also can be designed for integration into host chromosomes. Vectors capable of directing the expression of genes to which they are operatively linked are referred to herein as “expression vectors.” Expression vectors are often in the form of “plasmids,” which refer generally to circular double-stranded DNA loops which, in their vector form are not bound to the chromosome. “Plasmid” and “vector” are used interchangeably as the plasmid is the most commonly used form of vectors. Other such other forms of expression vectors that serve equivalent functions and that become known in the art subsequently hereto.

As used herein, vector also includes “virus vectors” or “viral vectors.” Viral vectors are engineered viruses that are operatively linked to exogenous genes to transfer (as vehicles or shuttles) the exogenous genes into cells.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include substitutions, deletions and insertions of nucleotides. An allele of a gene also can be a form of a gene containing a mutation.

As used herein, the terms “gene” or “recombinant gene” refer to a nucleic acid molecule containing an open reading frame and including at least one exon and, optionally, an intron-encoding sequence. A gene can be either RNA or DNA. Genes can include regions preceding and following the coding region (leader and trailer).

As used herein, “intron” refers to a DNA sequence present in a given gene which is spliced out during mRNA maturation.

As used herein, “nucleotide sequence complementary to the nucleotide sequence encoding the amino acid sequence set forth in SEQ ID NO:” refers to the nucleotide sequence of the complementary strand of a nucleic acid strand encoding a polypeptide that includes an amino acid sequence having the particular SEQ ID NO:. The term “complementary strand” is used herein interchangeably with the term “complement.” The complement of a nucleic acid strand can be the complement of a coding strand or the complement of a non-coding strand. When referring to double-stranded nucleic acids, the complement of a nucleic acid encoding a polypeptide containing amino acid residues having a sequence set forth in a particular SEQ ID NO: refers to the complementary strand of the strand encoding the amino acid sequence set forth in the particular SEQ ID NO: or to any nucleic acid molecule containing the nucleotide sequence of the complementary strand of the particular nucleic acid sequence. When referring to a single-stranded nucleic acid molecule containing a nucleotide sequence, the complement of this nucleic acid is a nucleic acid having a nucleotide sequence which is complementary to that of the particular nucleic acid sequence. As used herein, the term “coding sequence” refers to that portion of a gene that encodes a sequence of amino acids present in a protein.

As used herein, the term “coding sequence” refers to that portion of a gene that encodes a sequence of amino acids present in a protein.

As used herein, the term “sense strand” refers to that strand of a double-stranded nucleic acid molecule that has the sequence of the mRNA that encodes the sequence of amino acids encoded by the double-stranded nucleic acid molecule.

As used herein, the term “antisense strand” refers to that strand of a double-stranded nucleic acid molecule that is the complement of the sequence of the mRNA that encodes the sequence of amino acids encoded by the double-stranded nucleic acid molecule.

As used herein, an “array” refers to a collection of elements, such as nucleic acid molecules, containing three or more members. An addressable array is one in which the members of the array are identifiable, typically by position on a solid phase support or by virtue of an identifiable or detectable label, such as by color, fluorescence, electronic signal (i.e., RF, microwave or other frequency that does not substantially alter the interaction of the molecules of interest), bar code or other symbology, chemical or other such label. In certain embodiments, the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface.

As used herein, a “support” (e.g., a matrix support, a matrix, an insoluble support or solid support, etc.) refers to any solid or semisolid or insoluble support to which a molecule of interest (e.g., a biological molecule, organic molecule or biospecific ligand) is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacryl-amide, silicon, rubber, and other materials used as supports for solid phase syntheses, affinity separations and purifications, hybridization reactions, immunoassays and other such applications. The matrix herein can be particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads,” are often, but not necessarily, spherical. Such reference, however, does not constrain the geometry of the matrix, which can be any shape, including random shapes, needles, fibers, and elongated. Roughly spherical “beads,” particularly microspheres that can be used in the liquid phase, also are contemplated. The “beads” can include additional components, such as magnetic or paramagnetic particles (see, for example, Dynabeads (Dynal, Oslo, Norway)) for separation using magnets as long as the additional components do not interfere with the methods and analyses herein.

As used herein, matrix or support particles refer to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μmm³ or less and can be order of cubic microns. Such particles are collectively called “beads.”

As used herein, the abbreviations for any protective groups, amino acids and other compounds are, unless indicated otherwise, in accord with their common usage, recognized abbreviations, or the IUPAC-IUB Commission on Biochemical Nomenclature (1972) Biochem., 11: 942-944.

B. Interferon-Gamma (IFN-γ)

Cytokines regulate the initiation, maintenance and suppression of immune responses against foreign antigens and tumors. Sub-populations of immune cells produce particular cytokines. This regulation is mediated by CD4+ helper cells that are subdivided into distinct subsets based upon the type of cytokines they produce. Among these sub-populations are Th1 cells and Th2 cells. Each produce a characteristic array of cytokines. Th1 cells produce an array of cytokines, including IFN-γ. Th1 cytokines, such as IFN-γ, enhance cell-mediated immunity, inhibit humoral immunity and result in a protective effect against pathogens that are primarily removed through cell-mediated immunity. Th1 cells produce IFN-γ and promote cell-mediated immune responses and viral neutralizing antibody responses of the IgG2a isotype, while Th2 cells produce IL-4 and stimulate B-cell proliferation and differentiation promoting predominantly IgG1 and IgE antibody production. Progression of many diseases is associated with a switch from a Th1 to a Th2 profile. Administration of Th1 cytokines has been shown to have an effective protective and immunomodulatory effect.

Interferon-gamma (IFN-γ) is a lymphorme (cytokine) produced in animals, including mammals. In general, IFN-γ is produced by immune cells including activated T-cells and NK cells. IFN-γ, and the cells that produce it, play a pivotal role in host defense by exerting anti-viral, antiproliferative and immunoregulatory effects on a variety of cells types. Activity of the cytokine varies depending on the origin of the cell on which it acts. For example, anti-viral activity is observed on many cells of non-hematopoietic origin.

1. IFN-γ Structure and Function

IFN-γ polypeptides are heterogeneous polypeptides, are made of varying amino acid sequence lengths and include, but are not limited to, recombinantly produced polypeptide, synthetically produced polypeptide and IFN-γ extracted from cells and tissues such as, for example, T lymphocyte and natural killer (NK) cells. Generally, IFN-γ is produced as a larger polypeptide that is matured to a smaller polypeptide upon cleavage of the signal sequence. Mature IFN-γ polypeptides are typically 124-146, amino acids in length after cleavage of the signal sequence. The precursur form of human IFN-γ is a 166 amino acid polypeptide (for example, SEQ ID NOS: 370 and 371), and includes a signal sequence (i.e. amino acids 1-20) that is cleaved, resulting in a mature polypeptide, such as for example, a mature polypeptide of 146 amino acids set forth in SEQ ID NOS: 1 and 2, respectively. IFN-γ polypeptides from different species share conserved sequences of amino acids (see, for example, mammalian IFN-γ including gibbon, monkey, baboon, cow, mouse and hamster). Non-human polypeptides include, but are not limited to, those containing a sequence of amino acid sequences set forth in SEQ ID NOS: 380-399.

In addition, IFN-γ is a heterologous protein and exists in forms that are 124-146 amino acids in length such as, for example, 124, 125, 138, 139, 140, 141, 142, 143, 144, 146, 145, 147 and 148 amino acids in length. Such truncations include deletion of 1 up to 18 amino acids at the C terminus while still retaining some activity of the IFN-γ polypeptide. Exemplary IFN-γ polypeptides containing C-terminal truncations are set forth in SEQ ID NOS: 373-377. The heterogeneity of IFN-γ polypeptides stems from natural truncations of IFN-γ that occur at the C-terminus, for example due to proteolytic digestion before and after secretion. For example, the truncation can be effected by endo- and/or exoprotease activity produced by the host cell. In some instances, the proteolytic degradation after secretion is due to the presence of proteolytic enzymes in the culture medium.

Deletion of amino acid residues at the N-terminus of IFN-γ also have been reported. In some instances, deletion of residues at the N-terminus is due to post-translational modification. Depending on the source of IFN-γ, post-translational modification of IFN-γ can involve the removal of the first three amino acids from the mature polypeptide set forth in SEQ ID NO:1, i.e. amino acid residues Cys-Tyr-Cys (The Cytokine FactsBook, (1994) Callard R., and Gearing A., (eds) pp. 157-158). For example, a form of IFN-γ produced from mitogen induction of human peripheral blood lymphocytes is 143 amino acids (SEQ ID NO:379) and includes a deletion of the three N-terminal amino acids Cys-Tyr-Cys as compared to the IFN-γ polypeptide set forth in SEQ ID NO:1. Such an IFN-γ polypeptide can have a pyroglutamate residue at its amino terminus. Removal of the blocked N-terminal residue, pyroglutamate, from the polypeptide can be achieved by treating with pyroglutamate aminopeptidase.

In some instances, recombinant expression of an IFN-γ polypeptide, such as any IFN-γ polypeptide provided herein, in E. coli usually includes the addition of a start codon (AUG) to the N-terminus of a mature polypeptide, which specifies a methionine residue. Such an N-terminal methionine is required for expression in bacteria. Thus, for example, an IFN-γ polypeptide, such as is produced recombinantly, can contain an initial methionine when produced in hosts where the methionine is not intracellularly cleaved. An example of an IFN-γ polypeptide containing an initial methionine is set forth in SEQ ID NO:372.

Truncations of IFN-γ polypeptides vary depending on the method of production and host cell used. For example, truncation of an IFN-γ polypeptide can vary depending on production conditions including, but not limited to, such factors as medium, host cell, and purification conditions. Thus, how an IFN-γ polypeptide is processed is specific to the host cells used to produce the polypeptide. Typically, C-terminal truncation of the IFN-γ polypeptide occur when IFN-γ is produced in mammalian cell lines, however, truncation also can occur in other hosts, such as for example, E. coli. In some instances, a heterogeneous population of IFN-γ polypeptides is produced. For example, the secretion of recombinant IFN-γ from host cells, such as for example CHO cells, can result in heterogeneity due to molecular and structural variability of the polypeptide due to glycosylation heterogeneity, non-covalent multi-molecular formation (i.e. dimerization), and primary structure truncation due to proteolysis. In some instances, IFN-γ produced from CHO cells, for example, exhibits glycosylation heterogeneity whereby 50% of the IFN-γ polypeptide utilize both glycosylation sites, about 40% utilize one glycosylation site, and about 10% are not glycosylated (Hooker et al. (1998) J. Interferon and Cytokine Res. 18: 287-295; Sarenva et al. (1995) Biochem J., 308: 9-14).

For purposes herein, modification of an IFN-γ polypeptide is with reference to an IFN-γ polypeptide having a length of 146 amino acids. It is understood, however, that modified IFN-γ polypeptides provided herein can be produced by methods known to those of skill in the art and can be produced in varying lengths. Thus, the modified IFN-γ polypeptides provided herein include those that are 146 amino acids in length (i.e. containing amino acid modifications in a mature IFN-γ polypeptide set forth in SEQ ID NO: 1 or 2), fragments thereof that are variously truncated at the N- or C-terminus, or combinations thereof. If necessary, IFN-γ polypeptides can be purified to homogeneity to be of any desired length.

For example, methods can be used to obtain or enrich for a more homogeneous population of IFN-γ polypeptides, if necessary. In one example, protease inhibitors can be added to the culture media of host cells used for the expression and production of the polypeptide, in order to limit the proteolysis of the polypeptide that can occur upon secretion into the medium. In another example, purification techniques can be employed to obtain a homogenous population of IFN-γ polypeptides of the desired length. Such purification techniques are well known to those of skill in the art and include, but are not limited to, size exclusion chromatography. A homogenous population of IFN-γ also can be achieved by producing a polypeptide of 132 amino acids in length (corresponding to amino acids 4-135 of the polypeptide set forth in SEQ ID NO:1), see e.g., U.S. Patent Publication No. US 2005/0201982. Such a polypeptide does not undergo C-terminal truncation, or at least is not significantly C-terminally truncated. For example, such a polypeptide can be genetically engineered at the nucleotide level by introducing a stop codon after the codon encoding amino acid residue number 132. The production of an IFN-γ polypeptide of 132 amino acids in eukaryotic host cells, or modified forms thereof such as containing modifications provided herein, results in a homogeneously active polypeptide.

In an additional example, the glycosylation heterogeneity that can be observed in recombinantly produced IFN-γ polypeptides also can be reduced. It has been shown that modification of S102T, based on the numbering of the amino acid polypeptide set forth in SEQ ID NO:1, is required for the optimization of the in vivo N-glycosylation site at position N100 of IFN-γ (see e.g., US Patent Publication No. US 2005/0249703). Thus, any modification provided herein can optionally be combined with a modification of S102T, so as to ensure the higher utilization of the position 100 in vivo N-glycosylation site, resulting in a polypeptide that is more homogeneously glycosylated.

IFN-γ includes related polypeptides from different species including, but not limited to, animals of human and non-human origin, allelic variant isoforms, species isoforms, synthetic molecules from nucleic acids, protein isolated from human tissue and cells, and modified forms thereof, such as for example, truncated forms. Exemplary unmodified mature human IFN-γ polypeptides include, but are not limited to, unmodified and wild-type native IFN-γ polypeptide (such as the polypeptide containing a sequence set forth in SEQ ID NO: 1) and the unmodified and wild-type precursor IFN-γ polypeptide that includes a signal peptide (e.g., the polypeptide that has the sequence set forth in SEQ ID NO: 370), a polymorphic wild-type native IFN-γ polypeptide that has a mutation at Q140R (SEQ ID NO: 2; or its precursor containing a sequence of amino acids set forth in SEQ ID NO: 371). Other exemplary IFN-γ polypeptides include any of the variously truncated IFN-γ polypeptides set forth in any of SEQ ID NOS:372-379 or any of the IFN-γ species of polypeptides set forth in any of SEQ ID NOS: 380-389. Any of these forms can be modified as described herein. For example, a modified IFN-γ polypeptide provided herein include any of the above forms of IFN-γ that have a modification at a position corresponding to a Y2H modification in SEQ ID NO:1.

Distinct regions of the polypeptide participate in IFN-γ receptor binding. The C-terminus of IFN-γ has been implicated in the mediation of activities of the cytokine. Deletions in the C-terminus of IFN-γ can reduce the anti-viral and antiproliferative activities of IFN-γ. This reduction can be mediated in part by a loss of protein structure as well as loss of portions of the molecule that participate in biological function. IFN-γ contains two neutralizing epitopes that participate in anti-viral activity.

IFN-γ is functionally active as a homodimer of two monomer subunits. The monomeric subunits are non-covalently bound to each other in an anti-parallel fashion and form a globular structure. Each subunit contains six alpha helices and low amounts of beta pleated sheets. The dimeric structure is stabilized by the intertwining of helices across the subunit interfaces with multiple intra- (within monomers) and inter- (between monomers) subunit interactions. For purposes of explanation, the first monomer of the dimer is designated “A” and the second monomer of the dimer is designated “B,” whereby the six helices of each monomer are designated A1-A6 and B1-B6, respectively. Amino acids within the helices of each monomer interact with each other, thereby contributing to protein stability within each monomer (i.e., “intrastability”). In addition, interhelical contacts occur between helices of different subunits thereby contributing to protein stability between the monomers (i.e., “interstability”). For example, amino acid residues in helices on monomer A can interact with amino acid residues in helices on monomer B. Amino acid residues attributed to each helix vary slightly depending on the source of structural information. For example, in the broadest example, helices A1 and B1 contain residues 6-18 of the mature IFN-γ polypeptide, helices A2 and B2 contain residues 33-41 of the mature IFN-γ polypeptide, helices A3 and B3 are made up of residues 44-66 of the mature IFN-γ polypeptide, helices A4 and B4 are made up of residues 72-86 of the mature IFN-γ polypeptide, helices A5 and B5 are made up of residues 91-102 of the mature IFN-γ polypeptide and helices A6 and B6 are made up of residues 108-125 where the residues correspond to the positions of a mature IFN-γ polypeptide, such as a polypeptide having an amino acid sequence set forth in SEQ ID NO: 1 (Ealick et al. Science 252: 698 (1001)). In another example, helices A1 and B1 contain residues 7-18 of the mature IFN-γ polypeptide, helices A2 and B2 contain residues 35-36 of the mature IFN-γ polypeptide, helices A3 and B3 are made up of residues 48-62 of the mature IFN-γ polypeptide, helices A4 and B4 are made up of residues 72-84 of the mature IFN-γ polypeptide, helices A5 and B5 are made up of residues 95-99 of the mature IFN-γ polypeptide and helices A6 and B6 are made up of residues 109-117 where the residues correspond to the positions of a mature IFN-γ polypeptide, such as a polypeptide having an amino acid sequence set forth in SEQ ID NO: 1 (See UniProtKB/Swiss-Prot entry P01579). In yet another example, helices A1 and B1 contain residues 6-17 of the mature IFN-γ polypeptide, helices A2 and B2 contain residues 33-37 of the mature IFN-γ polypeptide, helices A3 and B3 are made up of residues 42-63 of the mature IFN-γ polypeptide, helices A4 and B4 are made up of residues 67-85 of the mature IFN-γ polypeptide, helices A5 and B5 are made up of residues 89-99 of the mature IFN-γ polypeptide and helices A6 and B6 are made up of residues 106-112 where the residues correspond to the positions of a mature IFN-γ polypeptide, such as a polypeptide having an amino acid sequence set forth in SEQ ID NO: 1.

IFN-γ is a secreted, glycosylated protein with primarily biantennary complex-type sugar chains (Fukuta et al. (2000) Glycobiology 10: 421-420). Two N-linked glycosylation sites are present at amino acid positions corresponding to N-28 and N-100 of SEQ ID NO: 1 (Saravena et al. (1994) Biochem J. 303: 831-840). Dimers of IFN-γ secreted from leukocytes include fully-glycosylated IFN-γ (4 sites) and lesser glycosylated forms (3, 2, 1 or no sites glycosylated). The extent of glycosylation can contribute to the stability of the protein to protease resistance (Saravena et al. (1995) Biochem J. 308: 9-14). Glycosylation also can contribute to the amount of protein expression, secretion and potency of IFN-γ (Fukuta et al. (2000) Glycobiology 10: 421-420).

IFN-γ is a pleotropic cytokine that regulates immune and inflammatory events. For example, IFN-γ has the ability to enhance the functional activity of macrophages, promote T and B cell differentiation, modulate class I and II MHC antigen expression on a variety of cells, activate natural killer cells and neutrophils and prolong neutrophil survival. IFN-γ stimulates macrophage microbicidal and tumoricidal activity by inducing production of cytokines (such as TNF-α, reactive oxygen and nitrogen intermediates, and indolamine 2,3-dioxygenase); promotes inflammatory responses; stimulates the formation of granulomas which are important in host defense against intracellular pathogens; and induces expression of Fc receptors, the leukocyte adhesion protein LFA1, endotoxin binding sites, and the receptor for the endotoxin-lipopolysaccharide-binding protein complex (CD14). IFN-γ also modulates CD4+ Th cell function by regulating IL-4, IL-5 and IL-10. IFN-γ activates endothelial cells, fibroblasts, epithelial cells, hepatocytes and macrophages to kill intracellular pathogens. Nitric oxide (NO) is induced via induction of pro-inflammatory cytokines, such as IFN-γ, and has become increasingly important in host defense mechanisms.

IFN-γ exerts its activity by binding to one of two forms of IFN-γ receptors at the cell surface: one form of the receptor is found on the surface of non-hematopoietic cells and the other is found on the surface of hematopoietic cells (Fisher et al. J. Biol. Chem. 263: 2632-2637 (1988)). IFN-γ receptors are 90 kDa single chain glycoproteins. The polypeptide chain (IFN-γR1) confers ligand binding and processing activity to cells. In addition to the single polypeptide chain, the IFN-γ receptor appears to require a second component in order to be functionally active. The second component (accessory chain) acts in an accessory manner and has been designated IFN-γR2 or AF-1 (Schreiber et al. Gastroenterol Jpn. 28 (Suppl. 4): 88-94; discussion 95-6 (1993)). This second component of the receptor participates in the mediation of the biological effects and signal transduction of the receptor. (Rhee et al. (1996) J. Biol. Chem. 271: 28947-52). The IFN-γ receptor complex participates with Janus kinases 1 and 2 (Jak-1 and Jak-2) to mediate signal transduction. These kinases associate with the intracellular regions of the receptor polypeptide chains. In response to the ligand (i.e., IFN-γ), IFN-γR1, Jak-1 and Jak-2 are phosphorylated.

Signaling by IFN-γ through its receptor requires Jak1 and Jak2 (i.e., tyrosine kinases) that exist in a pre-formed complex with the receptor before ligand binding. Binding of IFN-γ to its receptor leads to phosphorylation of the receptor and Jak1 and Jak2 on tyrosine residues, thereby activating them. Jak-kinase activation leads to rapid induction of tyrosine phosphorylation and dimerization of the latent transcription factor STAT-1α, and phosphorylated STAT-1α binds to the γ-activation sequence (GAS) of a number of IFN-γ primary and secondary response genes, such as, for example, class II MHC expression (Benveniste and Benos, FASEB J. 9: 1577-1584 (1995)).

2. IFN-γ as a Pharmaceutical

Activities induced by IFN-γ include, but are not limited to, anti-viral, anti-proliferative, anti-tumoricidal, anti-fungal, anti-protozoan, anti-microbial and anti-fibrotic activities. Administration of IFN-γ has been shown to enhance phagocyte functions and improve resistance to infection. IFN-γ is recognized as the most broadly acting anti-microbial-inducing and host defense-enhancing cytokine identified in animal models. In man, treatment with immunoenhancing doses of IFN-γ have been found to be safe and well-tolerated. IFN-γ stimulates the antimicrobial mechanisms of blood monocytes, circulating neutrophils and tissue macrophages and aerosol administration activates alveolar macrophages in a compartmentalized fashion. In patients with cancer, leprosy and AIDS, monocytes are activated by IFN-γ administration, and monocyte HLA-DR expression in trauma patients can be up-regulated (Murray, H W. Intensive Care Med. 22(Suppl 4): S456-461 (1996)). IFN-γ also is administered to subjects suffering from malignant osteopetrosis, an inherited disorder characterized by an osteoclast defect leading to bone overgrowth and deficient phagocyte oxidative mechanisms. IFN-γ administration has been shown to enhance osteoclast function and delay disease progression. Administration of IFN-γ to cancer patients prevents or slows tumor development (Ikeda et al. Cytokine & Growth Factor Reviews 13: 95-109 (2002)).

For example, progression of viral diseases is associated with a switch from a Th1 to a Th2 profile, and administration of Th1 cytokines (for example, IFN-γ) has been shown to have an effective protective and immunomodulatory effect. The inorganic radical nitric oxide (NO) has been implicated in host defense mechanisms of virus infections and is induced by pro-inflammatory cytokines, such as IFN-γ (Akaike et al. Proc Soc Exp Biol Med. 217(1):64-73 (1998)). In addition, IFN-γ is the most broadly-acting antimicrobial-inducing and host defense-enhancing cytokine identified in experimental models of infectious disease, and is well-tolerated in vivo. IFN-γ, stimulates the antimicrobial mechanisms of blood monocytes, circulating neutrophils and tissue macrophages. Protozoan parasites cause intracellular infections that take place primarily in macrophages. IFN-γ provides one of the major signals for activation of macrophages, which are required for killing parasitic protozoans.

Cellular immunity and a Th1 response also are involved in anti-fungal host defense. Cytokines and anti-cytokines that promote Th1 pathways can be protective in vivo and, additionally, can act synergistically with other anti-fungal drugs. IFN-γ is a prominent cytokine that exhibits anti-fungal activity and acts by stimulating macro-phages, monocytes and neutrophils. Macrophages and neutrophils play a role in the stasis or killing of these organisms by using oxygen radicals, cationic proteins, nitric oxide (NO) and peroxides or iron deprivation. Specifically, activation of macrophages and neutrophils during an acquired immune response in response to IFN-γ increases the capacity of these cells to control and kill mycoses infections. Opportunistic fungal pathogens are most detrimental to immunosuppressive subjects such as those taking immunosuppressive regimens prior to, and subsequent to, solid organ or bone marrow transplant, cancer patients, and immunosuppressed HIV patients. Thus, there is a need for immunotherapeutic agents such as IFN-γ that promote strong Th1 immune responses.

IFN-γ therapy can be used to treat a wide variety of cancers, including but not limited to, T cell lymphoma, melanomas, sarcomas, advanced renal cell carcinoma, primary brain tumors, ovarian carcinoma, prostate cancer, pancreatic cancer, basal cell carcinoma. Treatments include, but are not limited to, intraperitoneal and intravenous administration of IFN-γ. Cancer treatments include reduction of metastasis as well as treatment at tumor sites. Modes of administration include, but are not limited to, IFN-γ protein injection or administration of the nucleic acid molecules encoding modified IFN-γ polypeptides provided herein and also can include additional treatments administered similarly or through other routes of administration, including oral administration and inhalation, stem cell engraftment at tumor sites, administration of an adenovirus vector encoding an IFN-γ systemically and/or at the tumor site. IFN-γ also can be expressed in stem cells and stem cell engrafted at the tumor site used for targeted therapy. Patients rendered T cell-deficient by advanced disease due an underlying neoplastic disorder are vulnerable to opportunistic infections that often fail to respond to traditional therapies, but which do respond to immunotherapeutic approaches such as IFN-γ, that activate macrophages and monocytes or enhance T cell function (Murray, H W. Clin. Infec. Dis. 19 (Suppl 2): S407-S413 (1993)).

Osteoclasts are cells of monocyte/macrophage origin that erode bone matrix and regulation of their differentiation is central to the understanding of the pathogenesis and treatment of bone diseases such as osteopetrosis. Briefly, bone-resorbing osteoclasts and bone-forming osteoblasts are essential to maintaining a balance between bone resorption and formation. Malignant osteopetrosis (OP) is an inherited condition characterized by an osteoclast defect. The disease is characterized by bone overgrowth and deficient phagocyte oxidative metabolism. IFN-γ is administered to subjects suffering from malignant osteopetrosis, an inherited disorder characterized by an osteoclast defect leading to bone overgrowth and deficient phagocyte oxidative mechanisms. IFN-γ administration has been shown to enhance osteoclast function and delay disease progression by enhancing superoxide production by phagocytes. Additionally, IFN-γ treatment increases osteoclastic bone resorption in vivo in subjects with malignant osteopetrosis.

IFN-γ is used for the prophylaxis and treatment of chronic granulomatous disease in adults and children. Superoxide production and bacteriocidal activity of the leukocytes from some cases of chronic granulomatous disease are improved by injection of IFN-γ (Yata J. Nippon Rinsho. 50(8):1990-5 (1992)).

The immune system plays a central role in the development of most forms of pulmonary fibrosis. An excess of profibrotic cytokines and/or a deficiency in anti-fibrotic cytokines have been implicated in the pathogenical process of IPF, as has excessive oxidation. The goal of treatment is to decrease lung inflammation and subsequent scarring because once scarring has developed, it is permanent. In IPF, the cytokine pattern of the immune response shifts toward a Th2 type response, which causes lung fibrosis. Thus, Th1 cytokines such as IFN-γ are proective.

The use of IFN-γ in diseases or disorders associated with extreme levels of serum IgE derives from the ability of IFN-γ to regulate production of IgE by B cells. IFN-γ acts directly on differentiated B cells to induce production of antibody isoforms other than IgG4 or IgE. IFN-γ also is a potent inhibitor if IL-4 production.

A well-established link exists between respiratory infection and asthma exacerbation, and viral respiratory tract infections are a major cause of wheezing in infants and adult asthma patients which are greater than non-asthmatics. Asthma is characterized by a strong Th2 response, and the ability of IFN-γ to switch immune responses to Th1 responses has been established.

Allergic pathogenesis is caused by preferential activation of Th2 cells in pre-disposed individuals. Stimulation of allergen-specific T cells by allergen-derived peptides, presented by antigen presenting cells on class II MHC molecules results in differentiation of CD4+ T cells into Th2 cytokine producing cells which produce IL-4, IL-13 and IL-5 which regulate the allergic response (Wills-Karp et al. Nature Reviews Immunology 1: 69-75 (2001)). P Thus, IFN-γ can be used in the treatment of a variety of infections, including, for example, bacterial, viral, fungal and protozoal infections, cancer, bone over-development disorders, chronic granulomatous disease, idiopathic pulmonary fibrosis hyper-IgE states and diseases and disorders in which induction of cell-mediated immunity would be beneficial.

IFN-γ can be administered orally, systemically, buccally, transdermally, intravenously, intramuscularly and subcutaneously and, typically, multiple administrations are used in treatment regimens. The formulations are typically stored in refrigerated (2-8° C.) conditions to ensure retention of activity. Hence, improved IFN-γ stability (half-life) in administered conditions (in vivo ), such as stability in serum, and in vitro (e.g., during production, purification and storage conditions) can improve its utility and efficiency as a drug.

Provided herein are variants of the IFN-γ polypeptide that display improved stability as assessed by resistance to proteases (blood, intestinal, etc.) and/or increased thermal tolerance and/or pH conditions, wherein the mutant variants exhibit increased protein half-life. The mutant variants that exhibit improved stability possess increased stability in administration conditions such as in the bloodstream, gastrointestinal tract, under low pH conditions (e.g., the stomach), mouth, throat, and/or under storage conditions.

C. Exemplary Methods for Modifying IFN-65

Provided herein are methods for increasing stability and half-life of a polypeptide by increasing resistance to proteolysis and/or by increasing thermal tolerance. For example, among modifications of interest for therapeutic proteins are those that increase protection against protease digestion while maintaining the requisite activity. Such changes are useful for producing longer-lasting therapeutic proteins. Provided herein are methods of modifying polypeptides, such as IFN-γ, to increase resistance to proteolysis by proteases (blood, serum, gastrointestinal, etc.), whereby the modified polypeptide exhibits increased half-life in vitro and/or in vivo.

Provided herein are modified IFN-γ polypeptides that display improved stability as assessed by resistance to proteases and/or increased thermal tolerance; the modified polypeptides exhibiting these properties possess increased protein half-life in vitro or in vivo. Also provided herein are the modified polypeptides generated by said methods. P The modified IFN-γ polypeptides (also referred to herein as variants) are more stable compared to unmodified IFN-γ polypeptides. Increasing stability (i.e., the half-life of proteins in vivo) can result in a decrease in the frequency of injections needed to maintain a sufficient drug level in serum, thus leading to: i) higher comfort to, and acceptance by, treated subjects, particularly human subjects, ii) lower doses necessary to achieve comparable biological effects, and iii) as a consequence, an attenuation of the (dose-dependent) secondary effects.

Increased stability of IFN-γ can be achieved, for example, by destruction of protease target residues or sequences and/or (ii) by an increase in thermal tolerance and/or tolerance to pH and/or other denaturing conditions of the protein. Modification of IFN-γ to increase stability can be accomplished while keeping activity unchanged compared to the unmodified or wild-type IFN-γ. Alternatively, modification of IFN-γ stability can be accomplished while increasing activity compared to the unmodified or wild-type IFN-γ. Any methods known in the art can be used to create modified IFN-γ polypeptides. In the methods described herein, modifications are chosen using the method of 2D-scanning mutagenesis as described, for example, in PCT published applications WO 2004/022747 and WO 2004/022593.

In principle, there are several general approaches described for protein-directed evolution based on mutagenesis. Any of these, alone or in combination can be used to modify a polypeptide such as IFN-γ to achieve increased conformational stability and/or resistance to proteolysis. Such methods include random mutagenesis, where the amino acids in the starting protein sequence are replaced by all (or a group) of the 20 amino acids either in single or multiple replacements at different amino acid positions are generated on the same molecule, at the same time. Another method, restricted random mutagenesis, introduces either all of the 20 amino acids or DNA-biased residues. The bias is based on the sequence of the DNA and not on that of the protein in a stochastic or semi-stochastic manner, respectively, within restricted or predefined regions of the protein known in advance to be involved in the activity being “evolved.” Additionally, methods of rational mutagenesis including 1D-scanning, 2D-scanning and 3D-scanning can be used alone or in combination to construct modified IFN-γ or hIFN-γ variants.

1. 1D Scanning (“Rational Mutagenesis”)

Rational mutagenesis, also termed 1D-scanning, is a two-step process and is described in co-pending U.S. application Ser. No. 10/022,249 (U.S. Publication No. 2003/0134351-A1). Briefly, in the first step, full-length amino acid scanning is performed where each and every of the amino acids in the starting protein sequence, such as hIFN-γ (SEQ ID NOS: 1 and 2) is replaced by a designated reference amino acid (e.g., alanine). Only a single amino acid is replaced on each protein molecule at a time. These amino acid positions are referred to as HITs. In the second step, a new collection of molecules is generated such that each molecule differs from each of the others by the amino acid present at the individual HIT positions identified in step 1. All 20 amino acids (19 remaining) are introduced at each of the HIT positions identified in step 1; while each individual molecule contains, in principle, one and only one amino acid replacement. The newly generated mutants that lead to a desired alteration (such as an improvement) in a protein activity are referred to as LEADs. The methods permit, among other things, identification of new unpredicted sequences of amino acids at unpredicted regions along a protein to produce a protein that exhibits a desired altered activity compared to the starting protein. Further, because the selection of the target region (HITs and surrounding amino acids) for the second step is based upon experimental data on activity obtained in the first step no prior knowledge of protein structure and/or function is necessary.

2. 3D Scanning

3D scanning, as described in co-pending U.S. Published Application No. 2004-0132977-A1 and U.S. application Ser. No. 10/658,355 and published PCT applications WO 2004/022747 and WO 2004/022593, is an additional method of rational evolution of proteins based on the identification of potential target sites for mutagenesis (is-HITs). The method uses comparison of patterns of protein backbone folding between structurally related proteins, irrespective of the underlying sequences of the compared proteins. Once the structurally related amino acid positions are identified on the protein of interest, then suitable amino acid replacement criteria, such as PAM analysis, can be employed to identify candidate LEADs for construction and screening.

3. 2D-Scanning (Restricted Rational Mutagenesis)

The 2D-scanning (or restricted rational mutagenesis) methods for protein rational evolution (see, co-pending U.S. application Ser. No. 10/658,355 and U.S. Published Application No. US-2004-0132977-A1 and published International applications WO 2004/022593 and WO 2004/022747) are based on scanning over two dimensions. The first dimension is the amino acid position along the protein sequence, in order to identify is-HIT target positions. The second dimension is scanning the amino acid type selected for replacing a particular is-HIT amino acid position. An advantage of the 2D-scanning methods provided herein is that at least one, and typically the amino acid position and/or the replacing amino acid, can be restricted such that fewer than all amino acids on the protein-backbone are selected for amino acid replacement; and/or fewer than all of the remaining 19 amino acids available to replace an original, such as native, amino acid are selected for replacement.

Based on i) the particular protein properties to be evolved (e.g., resistance to proteolysis), ii) sequence of amino acids of the protein, and iii) the known properties of the individual amino acids, a number of target positions along the protein sequence are selected, in silico, as “is-HIT target positions.” This number of is-HIT target positions is as large as reasonably possible such that all reasonably possible target positions for the particular feature being evolved are included. In particular, embodiments where a restricted number of is-HIT target positions are selected for replacement, the amino acids selected to replace the is-HIT target positions on the particular protein being optimized can be either all of the remaining 19 amino acids or, more frequently, a more restricted group comprising selected amino acids that are contemplated to have the desired effect on protein activity. In another embodiment, so long as a restricted number of replacing amino acids are used, all of the amino acid positions along the protein backbone can be selected as is-HIT target positions for amino acid replacement. Mutagenesis then is performed by the replacement of single amino acid residues at specific is-HIT target positions on the protein backbone (e.g., “one-by-one,” such as in addressable arrays), such that each individual mutant generated is the single product of each single mutagenesis reaction. Mutant DNA molecules are designed, generated by mutagenesis and cloned individually, such as in addressable arrays, such that they are physically separated from each other and that each one is the single product of an independent mutagenesis reaction. Mutant protein molecules derived from the collection of mutant DNA molecules also are physically separated from each other, such as by formatting in addressable arrays. Thus, a plurality of mutant protein molecules is produced. Each mutant protein contains a single amino acid replacement at only one of the is-HIT target positions. Activity assessment is then individually performed on each individual protein mutant molecule, following protein expression and measurement of the appropriate activity. An example of practice of this method is shown in the Examples in which mutant hIFN-γ molecules are produced.

The newly generated proteins that lead to altered, typically improved, target protein activity are referred to as LEADs. This method relies on an indirect search for protein improvement for a particular activity (such as increased resistance to proteolysis), based on amino acid replacement and sequence change at single or, in another embodiment, a limited number of amino acid positions at a time. As a result, optimized proteins, which have modified sequences of amino acids at some regions along the protein that perform better (at a particular target activity or other property) than or different from the starting protein, are identified and isolated.

2D-scanning on IFN-γ was used to generate variants improved in protein stability, including improved resistance to proteolysis. To effect such modifications, amino acid positions were selected using in silico analysis of hIFN-γ.

a. Identifying In-Silico HITs

The 2D-scanning method for directed evolution of proteins includes identifying and selecting (using in silico analysis) specific amino acids and amino acid positions (referred to herein as is-HITs) along the protein sequence that are contemplated to be directly or indirectly involved in the feature being evolved. As noted, the 2D-scanning methods provided include the following two steps. The first step is an in silico search of a target sequence of amino acids of the protein to identify all possible amino acid positions that potentially can be targets for the activity being evolved. This is effected, for example, by assessing the effect of amino acid residues on the property(ies) to be altered on the protein, using any known standard software. The particulars of the in silico analysis is a function of the property to be modified.

Once identified, these amino acid positions or target sequences are referred to as “is-HITs” (in silico HITs). In silico HITs are defined as those amino acid positions (or target positions) that potentially are involved in the “evolving” feature, such as increased resistance to proteolysis or thermal tolerance. The discrimination of the is-HITs among all the amino acid positions in a protein sequence can be made based on the amino acid type at each position in addition to the information on the protein secondary or tertiary structure. In silico HITs constitute a collection of mutant molecules such that all possible amino acids, amino acid positions or target sequences potentially involved in the evolving feature are represented. No strong theoretical discrimination among amino acids or amino acid positions is made at this stage. In silico HIT positions are spread over the full length of the protein sequence. Single or a limited number of is-HIT amino acids are replaced at a time on the target IFN-γ protein, for example, a hIFN-γ protein.

A variety of parameters can be analyzed to determine whether or not a particular amino acid on a protein might be involved in the evolving feature, typically a limited number of initial premises (typically no more than 2) are used to determine the in silico HITs. For example, as described herein, to increase the stability of IFN-γ, including hIFN-γ, the first condition is the nature of the amino acids linked to stability of the molecule such as its potential participation in chemical bridges that can participate in stabilization of the molecule. The second premise is typically related to the specific position of those amino acids along the protein structure.

During the first step of identification of is-HITs according to the methods provided herein, each individual amino acid along the protein sequence is considered individually to assess whether it is a candidate for is-HIT. This search is done one-by-one and the decision on whether the amino acid is considered to be a candidate for a is-HIT is based on (1) the amino acid type; (2) the position in the protein and protein structure if known; and (3) the predicted interaction between that amino acid and its neighbors in sequence and space.

Is-HITs were identified for a number of properties of hIFN-γ that contribute to protein stability. These properties included 1) increasing saline (polar) interactions between helices; 2) increasing H-bond interactions between helices and 3) removal of protease sensitive sites.

b. Identifying Replacing Amino Acids

Once the is-HITs target positions are selected, the next step is identifying those amino acids that will replace the original, such as native, amino acid at each is-HIT position to alter the activity level for the particular feature being evolved. The set of replacing amino acids to be used to replace the original, such as native, amino acid at each is-HIT position can be different and specific for the particular is-HIT position. The choice of the replacing amino acids takes into account the need to preserve the physicochemical properties such as hydrophobicity, charge and polarity of essential (e.g., catalytic, binding, etc.) residues and alter some other property of the protein (e.g., protein stability). The number of replacing amino acids of the remaining 19 non-native (or non-original) amino acids that can be used to replace a particular is-HIT target position ranges from 1 up to about 19 and anywhere in between depending on the properties for the particular modification.

Numerous methods of selecting replacing amino acids (also referred to herein as “replacement amino acids”) are well known in the art. Protein chemists determined that certain amino acid substitutions commonly occur in related proteins from different species. As the protein still functions with these substitutions, the substituted amino acids are compatible with protein structure and function. Often, these substitutions are to a chemically similar amino acid, but other types of changes, although relatively rare, also can occur.

Knowing the types of changes that are most and least common in a large number of proteins can assist with predicting alignments and amino acid substitutions for any set of protein sequences. Amino acid substitution matrices are used for this purpose. A number of matrices are available. A detailed presentation of such matrices can be found in the co-pending U.S. application Ser. No. 10/658,355 and U.S. Published Application No. US-2004-0132977-A1 and published International applications WO 2004/022593 and WO 2004/022747, each of which is incorporated herein in their entirety (where permitted). Such matrices also are known and available in the art, for example in the reference listed below.

In amino acid substitution matrices, amino acids are listed horizontally and vertically, and each matrix position is filled with a score that reflects how often one amino acid would have been paired with the other in an alignment of related protein sequences. The probability of changing amino acid “A” into amino acid “B” is assumed to be identical to the reverse probability of changing “B” into “A”. This assumption is made because, for any two sequences, the ancestor amino acid in the phylogenetic tree is usually not known. Additionally, the likelihood of replacement should depend on the product of the frequency of occurrence of the two amino acids and on their chemical and physical similarities. A prediction of this model is that amino acid frequencies will not change over evolutionary time (Dayhoff et al., Atlas of Protein Sequence and Structure, 5(3): 345-352, 1978). Several exemplary amino acid substitution matrices, including, but not limited to block substitution matrix (BLOSUM) (Henikoff et al., Proc. Natl. Acad. Sci. USA, 89: 10915-10919 (1992)), Jones et al. (Comput. Appl. Biosci., 8: 275-282 (1992)), Gonnet et al. (Science, 256: 1433-1445 (1992)), Fitch (J. Mol. Evol., 16(1): 9-16 (1966)), Feng et al. (J. Mol. Evol., 21: 112-125 (1985)), McLachlan (J. Mol. Biol., 61: 409-424 (1971)), Grantham (Science, 185: 862-864 (1974)), Miyata (J. Mol. Evol., 12: 219-236 (1979)), Rao (J. Pept. Protein Res., 29: 276-281 (1987)), Risler (J. Mol. Biol., 204: 1019-1029 (1988)), Johnson et al (J. Mol. Biol., 233: 716-738 (1993)), and Point Accepted Mutation (PAM) (Dayhoff et al., Atlas Protein Seq. Struct. 5: 345-352 (1978)).

The outcome of the two steps set forth above, which is performed in silico is that: (1) the amino acid positions that are the target for mutagenesis are identified (referred to as is-HITs); and (2) the replacing amino acids for the original, such as native, amino acids at the is-HITs are identified, to provide a collection of candidate LEAD mutant molecules that are expected to perform differently from the native molecule. These are assayed for a desired optimized (or improved or altered) activity.

c. Construction of Mutant Proteins and Biological Assays

Once is-HITs are selected as set forth above, replacing amino acids are introduced. Mutant proteins typically are prepared using recombinant DNA methods and assessed in appropriate biological assays for the particular activity (feature) optimized. An exemplary method of preparing the mutant proteins is by mutagenesis of the original, such as native, gene using methods well known in the art. Mutant molecules are generated one-by-one, such as in addressable arrays, such that each individual mutant generated is the single product of each single and independent mutagenesis reaction. Individual mutagenesis reactions are conducted separately, such as in addressable arrays where they are physically separated from each other. Once a population of sets of nucleic acid molecules encoding the respective mutant proteins is prepared, each is separately introduced one-by-one into appropriate cells for the production of the corresponding mutant proteins. This also can be performed, for example, in addressable arrays where each set of nucleic acid molecules encoding a respective mutant protein is introduced into cells confined to a discrete location, such as in a well of a multi-well microtiter plate. Each individual mutant protein is individually phenotypically characterized and performance is quantitatively assessed using assays appropriate for the feature being optimized (i.e., feature being evolved). Again, this step can be performed in addressable arrays. Those mutants displaying a desired increased or decreased performance compared to the original, such as native molecules are identified and designated LEADs. From the beginning of the process of generating the mutant DNA molecules up through the readout and analysis of the performance results, each candidate LEAD mutant is generated, produced and analyzed individually, such as from its own address in an addressable array. The process is amenable to automation.

D. Modified IFN-γ Polypeptides

Provided herein are variants of IFN-γ (also referred to herein as modified IFN-γ polypeptides) that display improved stability as assessed by resistance to proteases (blood, intestinal, etc.) and/or increased thermal tolerance and/or tolerance to pH conditions. Such variants can have an increased protein half-life in vitro (e.g., during production, purification and storage) and in vivo (e.g., after administration to a subject). In one embodiment, the modified IFN-γ polypeptides provided herein confer at least comparable activity. For example, the modified IFN-γ polypeptides confer at least comparable activity as assessed by IFN-γ-specific cell proliferation, anti-viral activity and/or bioactivity assay compared to the unmodified and/or wild-type native IFN-γ polypeptide. In some instances, the activity of a modified IFN-γ polypeptide is increased or is decreased as compared to an unmodified IFN-γ.

Modified IFN-γ polypeptides provided herein include modified human IFN-γ (hIFN-γ) polypeptides that have been modified at one or more than one amino acid as compared to an unmodified IFN-γ polypeptide. Such polypeptides contain a single mutation compared to a native polypeptide, or contain 2, 3, 4, 5, 6, 7, 8, 9, 10, 11, 12, 13, 14, 15, 16, 17, 18, 19, 20, or more mutations compared to a unmodified polypeptide. In some examples, modified IFN-γ polypeptides provided herein are variants of native IFN-γ. Exemplary unmodified human IFN-γ polypeptides have amino acid sequences set forth in SEQ ID NOS: 1 and 2, or fragments thereof. The hIFN-γ polypeptide can be of any human tissue or cell-type origin. Modified IFN-γ polypeptides provided herein also include variants of IFN-γ of non-human origin. For example, modified IFN-γ polypeptides can be variants of a mammalian IFN-γ, including, cow, sheep, dog, rat, mouse, hamster, rabbit, primate such as monkey, baboon, gibbon, macaque, gorilla, orangutan and chimpanzee, giant panda, marmoset, camel, llama, red deer, horse, bottle-nose dolphin and pig IFN-γ. Exemplary unmodified non-human IFN-γ polypeptides have amino acid sequences set forth in SEQ ID NOS: 380-399. Modified IFN-γ polypeptides also include polypeptides that are hybrids of different IFN-γ polypeptides and also synthetic IFN-γ polypeptides prepared recombinantly or synthesized or constructed by other methods known in the art based upon known polypeptides.

The modified IFN-γ polypeptides provided herein are altered in specific structural features that contribute to protein stability. In particular, such features are altered so that the polypeptides are more stable. The modified IFN-γ polypeptides provided herein include variants that possess increased resistance to proteolysis and/or increased thermal tolerance and/or stability to particular pH conditions. Hence, the IFN-γ variants provided herein offer IFN-γ polypeptides with advantages including a decrease in the frequency of injections needed to maintain a sufficient drug level in serum, thus leading to, for example, higher comfort and acceptance by subjects, lower doses necessary to achieve comparable biological effects, and attenuation of secondary effects.

Structural modifications can be made in IFN-γ by amino acid replacements to increase the conformational stability of IFN-γ. Mutations of any one or more than one amino acid residues in an IFN-γ polypeptide provided herein confer increased protein stability by virtue of a change in the primary sequence of the polypeptide. Other modifications that are or are not in the primary sequence of the polypeptide also can be included, such as, but not limited to, the addition of a carbohydrate moiety due to glycosylation of the polypeptide, the addition of polyethylene glycol (PEG) moiety to the polypeptide, and other such modifications. Typically, modifications provided herein include those that increase the protein stability of IFN-γ while either improving or maintaining the requisite activity (e.g., cell proliferation activity and/or anti-viral activity). These modifications can result in IFN-γ variants with improved stability as assessed by resistance to proteases (blood, intestinal, etc.) and/or increased conformational stability as assessed by increased thermal tolerance and/or resistance to pH conditions. Such modified IFN-γ polypeptides exhibit increased protein half-life compared to native IFN-γ in vitro and/or in vivo. These modifications include destruction of target sequences of amino acids sensitive to proteolysis, modification of hydrophobic patches to increase polar interactions with solvent and increasing polar interactions between particular helices of IFN-γ, and modifications to increase stability around glycosylation sites.

Structural modifications in IFN-γ include combining one, two or more amino acid replacements at different positions within the IFN-γ polypeptide to increase the stability of the IFN-γ polypeptide. Such combinations can be used to improve stability as assessed by resistance to proteases (blood, intestinal, etc.) and/or increased thermal tolerance or tolerance to other denaturing and/or stability disrupting conditions. For example, two or more modifications in one or more categories can be combined, where the categories are selected from, for example, disruption of target sequences sensitive to proteolysis, modification of hydrophobic patches to increase polar interactions with solvent, increasing polar interactions between helices and increasing interactions between helices. In addition, one or more modifications in such categories can be combined with one or more modifications of any known type to increase IFN-γ stability, for example, modifications which remove protease sensitive sites in IFN-γ with modifications of any known type that increase thermal tolerance. Thus, also among the variants provided herein are modified IFN-γ polypeptide with two or more modifications compared to native or wild-type IFN-γ. Modified IFN-γ polypeptides include those with 2, 3, 4, 5, 6, 7, 8, 9, 10, 11, 12, 13, 14, 15, 16, 17, 18, 19, 20 or more modified positions. The two or more modifications can include two or more modifications of the same property, e.g., two modifications that modify IFN-γ (i.e. hIFN-γ) thermal tolerance or two modifications that modify resistance to proteases (blood, intestinal, etc.). In another embodiment, the two or more modifications include combinations of properties that each contribute to IFN-γ stability. For example, an IFN-γ variant can include one or more modifications that alters IFN-γ thermal tolerance and one or more modifications that remove a protease sensitive site. IFN-γ variants carrying replacements at more than one is-HIT sites and displaying improved stability are called super-LEADs. An IFN-γ super-LEAD can for example, contain 2, 3, 4, 5, 6, 7, 8, 9, 10 or more than 10 amino acid changes compared to wild-type or unmodified IFN-γ.

Among the modified IFN-γ polypeptides provided herein are IFN-γ variants modified to: 1) increase interactions between helices; 2) increase interactions between helices of monomers in the IFN-γ dimer; and 3) remove protease sensitive sites. Also included among modified IFN-γ polypeptides provided herein are those IFN-γ variants modified to increase stability of the polypeptide around glycosylation sites. Particular of modifications of IFN-γ polypeptides provided herein are modifications of IFN-γ that have increased stability by increasing the resistance of the modified IFN-γ to proteolysis or increasing the tolerance to temperature or other denaturing agent.

In one embodiment, the regions selected for modification include one or more amino acid modifications in a region corresponding to positions in mature human interferon-γ selected from among amino acid residues corresponding to amino acid positions 1 to 32, 34-36, 38-39, 43-57, 59, 60, 62-63 and 67-146 of interferon-γ set forth in SEQ ID NO: 1. In some examples, the positions selected for modification include one or more amino acid modifications corresponding to amino acid positions 2, 5-12, 14-18, 24, 28-29, 31, 32, 35, 36, 39, 44, 45, 48, 51, 53-55, 57, 59, 60, 62, 63, 71-80, 83, 84, 89-99, 100, 101, 105, 106, 109, 115, 117, 122, 123, 125, 128, 131-134, 137-139, 142, 143 of interferon-γ set forth in SEQ ID NO: 1. In other examples, the positions selected for modification include one or more amino acid modifications corresponding to amino acid positions 2, 7, 8, 10, 11, 14, 16, 27, 35, 36, 39, 44, 45, 51, 53, 54, 59, 72, 76, 80, 91, 92, 94, 98, 115, 117, 123, 125, 128, 131, 132, 134, 137, 138 and 142 of a mature interferon-γ polypeptide set forth in SEQ ID NO:1.

In one embodiment, an IFN-γ polypeptide is modified to include one or more single amino acid replacements compared with unmodified IFN-γ polypeptide, where the replacement positions are not positions that correspond to positions 9, 29, 33, 37, 40, 41, 42, 58, 61, 64-66, 86, 88, 124, and 140 of SEQ ID NO: 1. In one embodiment, such modified IFN-γ polypeptide include a single amino acid replacement compared with the unmodified IFN-γ, where if position 9 is replaced, the replacing amino acid is not Q; if position 28 is replaced, the replacing amino acid is not A, H or S; if position 86 or 88 is replaced, the replacing amino acid is not D; if position 124 is replaced, the replacing amino acid is not P; if position 128 is replaced, the replacing amino acid is not E; or if position 140 is replaced, the replacing amino acid is not R in the amino acid sequence represented by SEQ ID NO: 1.

In another embodiment, an IFN-γ polypeptide is modified to include one or more single amino acid replacements compared with unmodified IFN-γ polypeptide, where the replacement positions are not positions that correspond to positions 5, 6, 9, 12, 15, 17, 18, 24, 28, 29, 31, 32, 48, 55, 57, 60, 62, 63, 71, 73, 74, 75, 77, 78, 83, 84, 89, 90, 93, 95, 96, 97, 100, 101, 105, 106, 109, 122, 133, 139, or 143 of SEQ ID NO:1. In one embodiment, modified IFN-γ polypeptides include single amino acid replacements so long as if positions 5, 6, 12, 17, 24, 62, 71, 74, 75, 77, 78, 89, 93, 96, 105, or 106 are replaced, the replacing amino acid is not cysteine; if position 9 or 28 is replaced, the replacing amino acid is not glutamine or cysteine; if position 15, 83 or 90 is replaced, the replacing amino acid is not serine, cysteine, or threonine; if position 29 is replaced, the replacing amino acid is not phenylalanine, asparagine, tyrosine, glutamine, valine, alanine, methionine, isoleucine, lysine, arginine, threonine, histidine, cysteine, or serine; if position 31 is replaced, the replacing amino acid is not histidine, aspartic acid, alanine, methionine, asparagine, threonine, arginine, serine, or cysteine; if position 18, 32, 55, 57, 60, 63, 84, 95, or 139 is replaced, the replacing amino acid is not valine; if position 48, 73, or 143 is replaced, the replacing amino acid is not asparagine; if position 97 or 122 is replaced, the replacing amino acid is not asparagine or cysteine; if position 100 is replaced, the replacing amino acid is not glutamine; if position 101 is replaced, the replacing amino acid is not phenylalanine, asparagines, glutamine, valine, alanine, methionine, isoleucine, lysine, glycine, arginine, threonine, histidine, cysteine, or serine; if position 109 is replaced, the replacing amino acid is not serine or threonine; and if position 133 is replaced, the replacing amino acid is not asparagine.

In one embodiment, an IFN-γ polypeptide is modified to include one or more single amino acid replacements compared with the unmodified IFN-γ, where the replacement positions are not positions that correspond to positions 33, 37, 40, 41, 42, 58, 61 and 64-66 of SEQ ID NO: 1. In another embodiment, an IFN-γ is modified to include one or more single amino acid replacements compared with the unmodified IFN-γ, where the replacement positions are not positions that correspond to positions represented by the sequence of amino acids of any of SEQ ID NOS: 39, 40, 43, 44, 47-54, 61, 62, 67, 68, and 71-76. For example, a modified IFN-γ polypeptide does not include a polypeptide modified by single amino acid replacement of the amino acid at position L33 by I or V such as is described in SEQ ID NOS: 39 and 40, respectively; or by replacement of the amino acid at position K37 by N or Q such as is described in SEQ ID NOS: 43 and 44, respectively; or by replacement of the amino acid at position K40 by N or Q such as is described in SEQ ID NOS: 47 and 48, respectively; or by replacement of the amino acid at position E41 by Q, H, or N such as is described in SEQ ID NOS: 49, 50, and 51, respectively; or by replacement of the amino acid at position E42 by Q, H, or N such as is described in SEQ ID NOS: 52, 53, and 54, respectively; or by replacement of the amino acid at position K58 by N or Q such as is described in SEQ ID NOS: 61 and 62, respectively; or by replacement of the amino acid at position K61 by N or Q such as is described in SEQ ID NOS: 67 and 68, respectively; or by replacement of the amino acid at position K64 by N or Q such as is described in SEQ ID NOS: 71 and 72, respectively; or by replacement of the amino acid at position D65 by N or Q such as is described in SEQ ID NOS: 73 and 74, respectively; or at by replacement of the amino acid at position D66 by N or Q such as is described in SEQ ID NOS: 75 and 76, respectively.

In one example, the modified IFN-γ polypeptides that exhibit increased protein stability have amino acid replacement(s) corresponding to any one or more amino acid positions including any of positions Y2, D5, P6, Y7, V8, K9, E10, A11, E12, L14, K15, K16, Y17, F18, D24, D27, N28, G29, L31, F32, I35, L36, W39, D44, R45, M48, Q51, V53, S54, F55, F57, L59, F60, N62, F63, K71, S72, V73, E74, T75, I76, K77, E78, D79, M80, K83, F84, K89, K90, K91, R92, D93, D94, F95, E96, K97, L98, T99, N100, Y101, D105, L106, Q109, E115, I117, E122, L123, P125, K128, K131, R132, K133, R134, M137, L138, F139, R142 and R143 of a mature human interferon-γ polypeptide. Exemplary of amino acid replacements at such positions include amino acid replacement(s) corresponding to any one or more of Y2H, Y2I, D5N, D5Q, P6A, P6S, Y7H, Y7I, Y7E, Y7D, Y7K, Y7R, Y7N, Y7Q, Y7S, Y7T, V8E, V8D, V8K, V8R, V8N, V8Q, V8S, V8T, K9N, E10Q, E10H, E10N, A11E, A11D, A11K, A11R, A11N, A11Q, A11S, A11T, E12Q, E12H, E12N, L14I, L14V, L14E, L14D, L14K, L14R, L14N, L14Q, L14S, L14T, K15N, K15Q, K16N, K16Q, Y17H, Y17I, Y17E, Y17D, Y17K, Y17R, Y17N, Y17Q, Y17S, Y17T, F18I, F18E, F18D, F18K, F18R, F18N, F18Q, F18S, F18T, D24N, D24Q, D27N, D27Q, N28S, G29P, L31I, L31V, F32I, I35E, I35D, I35K, I35R, I35N, I35Q, I35S, I35T, L36I, L36V, L36E, L36D, L36K, L36R, L36N, L36Q, L36S, L36T, W39H, W39S, D44N, D44Q, R45H, R45Q, M48E, M48D, M48K, M48R, M48Q, M48S, M48T, Q51E, Q51D, Q51K, Q51R, V53E, V53D, V53K, V53R, V53N, V53Q, V53S, V53T, S54E, S54D, S54K, S54R, S54N, S54Q, S54T, F55E, F55D, F55K, F55R, F55N, F55Q, F55S, F55T, F57I, F57E, F57D, F57K, F57R, F57N, F57Q, F57S, F57T, L59I, L59V, L59E, L59D, L59K, L59R, L59N, L59Q, L59S, L59T, F60I, F60E, F60D, F60K, F60R, F60N, F60Q, F60S, F60T, N62E, N62D, N62K, N62R, F63I, K71N, K71Q, S72E, S72D, S72K, S72R, S72N, S72Q, S72T, V73E, V73D, V73K, V73R, V73Q, V73S, V73T, E74Q, E74H, E74N, T75E, T75D, T75K, T75R, T75N, T75Q, T75S, I76E, I76D, I76K, I76R, I76N, I76Q, I76S, I76T, K77N, K77Q, E78Q, E78H, E78N, D79N, D79Q, M80E, M80D, M80K, M80R, M80N, M80Q, M80S, M80T, K83N, K83Q, F84I, F84E, F84D, F84K, F84R, F84N, F84Q, F84S, F84T, K89N, K89Q, K90N, K90Q, K91N, K91Q, R92H, R92Q, D93N, D93Q, D94N, D94Q, F95I, F95E, F95D, F95K, F95R, F95N, F95Q, F95S, F95T, E96Q, E96H, E96N, K97Q, L98I, L98V, L98E, L98D, L98K, L98R, L98N, L98Q, L98S, L98T, T99E, T99D, T99K, T99R, T99N, T99Q, T99S, N100A, D105N, D105Q, L106I, L106V, Q109E, Q109D, Q109K, Q109R, Q109N, E115Q, E115H, E115N, I117E, E117D, I117K, I117R, I117N, I117Q, I117S, I117T, E122Q, E122H, L123I, L123V, P125A, P125S, K128N, K128Q, K131N, K131Q, R132H, R132Q, K133Q, R134H, R134Q, M137I, M137V, L138I, L138V, F139I, R142H, R142Q, R143H, and R143Q.

In another embodiment, the positions modified correspond to modification of more than one amino acid position, e.g., 2, 3, 4, 5, 6, 7, 8, 9, 10, 11, 12, 13, 14, 15, 16, 17, 18, 19, 20 or more positions. In one embodiment, such modified IFN-γ include two amino acid replacements compared with the unmodified IFN-γ, where the replacement positions are not positions that correspond to combination mutants K9Q-Q140R, N86-Q140R, N88D-Q140R, K128E-Q140R or K133T-Q140R, wherein the IFN-γ has an amino acid sequence represented by SEQ ID NO: 1.

1. Increased Resistance to Proteolysis by Removal of Proteolytic Sites

Among modifications of interest for therapeutic proteins are those that increase protection against protease digestion while maintaining the requisite activity. Such changes are useful for producing longer-lasting therapeutic proteins. The delivery of stable peptide and protein drugs to patients is a major challenge for the pharmaceutical industry. These types of drugs in the human body are constantly eliminated or taken out of circulation by different physiological processes including internalization, glomerular filtration and proteolysis. The latter is often the limiting process affecting the half-life of proteins used as therapeutic agents in per-oral administration and either intravenous or intramuscular injections. Thus, in one aspect, the polypeptides provided herein have been modified to increase resistance to proteolysis, thereby increasing the half-life of the modified polypeptide in vitro (e.g., production, processing, storage, assay, etc.) or in vivo (e.g., serum stability). Thus, the modified polypeptides provided herein are useful as longer-lasting therapeutic proteins.

a. Proteases

Proteases, proteinases or peptidases catalyze the hydrolysis of covalent peptidic bonds. Proteases to which resistance is increased, include those that occur, for example, in body fluids and tissues, such as those that include, but are not limited to, saliva, blood, serum, intestinal, stomach, blood, cell lysates, cells and others. These include proteases of all types, such as, for example, serine proteases and matrix metalloproteinases.

i. Serine Proteases

Serine proteases participate in a range of functions in the body, including blood clotting, inflammation as well as digestive enzymes in both prokaryotes and eukaryotes. Serine proteases are sequence specific. While cascades of protease activations control blood clotting and complement, other proteases are involved in signaling pathways, enzyme activation and degradative functions in different cellular or extracellular compartments.

Serine proteases include, but are not limited, to chymotrypsin, trypsin, elastase, NS3, elastase, factor Xa, Granzyme B, thrombin, trypsin, plasmin, urokinase, tPA and PSA. Chymotrypsin, trypsin and elastase are synthesized by the pancreatic acinar cells, secreted in the small intestine and are responsible for catalyzing the hydrolysis of peptide bonds. All three of these enzymes are similar in structure, as shown through their X-ray structures. Each of these digestive serine proteases targets different regions of the polypeptide chain, based upon the amino acid residues and side chains surrounding the site of cleavage. The active site of serine proteases is shaped as a cleft where the polypeptide substrate binds. Amino acid residues are labeled from N to C term of the polypeptide substrate (Pi, . . . , P3, P2, P1, P1′, P2′, P3′, . . . , Pj) and their respective binding sub-sites (Si, . . . , S3, S2, S1, S1′, S2′, S3′, . . . , Sj). The cleavage is catalyzed between P1 and P1′. Chymotrypsin is responsible for cleaving peptide bonds flanked with bulky hydrophobic amino acid residues. Particular residues include phenylalanine, tryptophan and tyrosine, which fit into a snug hydrophobic pocket. Trypsin is responsible for cleaving peptide bonds flanked with positively charged amino acid residues. Instead of having the hydrophobic pocket of the chymotrypsin, there exists an aspartic acid residue at the back of the pocket. This can then interact with positively charged residues such as arginine and lysine. Elastase is responsible for cleaving peptide bonds flanked with small neutral amino acid residues, such as alanine, glycine and valine. The pocket that is in trypsin and chymotrypsin is now lined with valine and threonine, rendering it a mere depression, which can accommodate these smaller amino acid residues. Serine proteases are ubiquitous in prokaryotes and eukaryotes and serve important and diverse biological functions such as hemostasis, fibrinolysis, complement formation and the digestion of dietary proteins.

ii. Matrix Metalloproteinases

Matrix metalloproteinases (MMPs) are a family of Zn⁺²- and calcium-dependent endopeptidases that degrade components of the extracellular matrix (ECM). In addition, MMPs also can process a number of cell-surface cytokines, receptors and other soluble proteins. They are involved in normal tissue remodeling processes such as wound healing, pregnancy and angiogenesis. Under physiological conditions, MMPs are made as inactive precursors (zymogens) and are processed to their active form. Additionally, the enzymes are specifically regulated by endogenous inhibitors called tissue inhibitors of matrix metalloproteinases (TIMPs). The proteolytic activity of MMPs acts as an effector mechanism of tissue remodeling in physiologic and pathologic conditions, and as modulator of inflammation. The excess synthesis and production of these proteins lead to accelerated degradation of the ECM which is associated with a variety of diseases and conditions such as, for example, bone homeostasis, arthritis, cancer, multiple sclerosis and rheumatoid arthritis. In the context of neuroinflammatory diseases, MMPs have been implicated in processes such as (a) blood-brain barrier (BBB) and blood-nerve barrier opening, (b) invasion of neural tissue by blood-derived immune cells, (c) shedding of cytokines and cytokine receptors, and (d) direct cellular damage in diseases of the peripheral and central nervous system (Leppert et al. Brain Res. Rev. 36(2-3): 249-57 (2001); Borkakoti et al. Prog. Biophys. Mol. Biol. 70(1): 73-94 (1998)).

The activation of ubiquitous plasminogen by urokinase (u-Pa) and tissue-type plasminogen activator (t-Pa) is an initiator of the activation cascade of four classes of MMPs: collagenases, stromelysin, membrane-type metalloproteinases and gelatinases (Cuzner and Opdenakker. J. Neuroimmunol. 94(1-2): 1-14 (1999)). Proteolytic activities of MMPs and plasminogen activators, and their inhibitors, are important in maintaining the integrity of the ECM as cell-ECM interactions influence and mediate a wide range of processes including proliferation, differentiation, adhesion and migration of a variety of cell types. Excessive production of matrix metalloproteinases have been implicated in tissue damage and wound healing, inflammatory disorders, proliferative disorders and autoimmune diseases. (St-Pierre et al. Curr. Drug Targets Inflamm. Allergy 2(3): 206-215 (2003); Opdenakker, G. Verh. K. Acad. Geneeskd. Belg. 59(6): 489-514 (1997)).

Gelatinase B (MMP-9; type IV collagenase) belongs to a sub-family of MMPs that plays an important role in tissue remodeling in normal and pathological inflammatory processes and is the terminal member of the protease cascade which leads to matrix degradation. It cleaves gelatins and other substrates and is involved in matrix remodeling during embryogenesis, tissue remodeling and development. Gelatinase B is secreted by a variety of leukocytes including neutrophils, macrophages, lymphocytes, and eosinophils. Generally, the expression of gelatinase B is regulated, however, neutrophils store gelatinase B in secretory granules for rapid release. The expression of gelatinase B in cells can be induced by a variety of inflammatory stimuli including interleukin-1β, tumor necrosis factor-α, lymphotoxin, interferon beta, and lipopolysaccharides (LPS), and by other agents stimulating cell migration. For example, gelatinase B is up-regulated in pathological states such as invasion of cancer cells and when leukocytes are released from the bone marrow and migrate toward an inflammatory event. After stimulation by inflammatory cytokines, or upon delivery of bi-directional activation signals following integrin-mediated cell-cell or cell-ECM contact, gelatinase B also can be secreted by lymphocytes and stromal cells.

Gelatinase B, like other gelatinases and MMPs, is secreted in a latent inactive form and is converted to an active species by other proteolytic enzymes, including other MMPs. For example, activated gelatinase A can activate progelatinase B in a process that is inhibited by TIMP-1 and TIMP-2 (Fridman et al, Cancer Research, 55:2548-2555 (1995). Progelatinase B also can be converted to an active form via an interacting protease cascade involving plasmin and stromelysin-1 (MMP-3). Plasmin, generated by the endogenous plasminogen activator (uPA), is not an efficient activator of progelatinase B. Plasmin is able to generate active stromelysin-1 from an inactive proform and the activated stromelysin-1 is itself a potent activator of gelatinase B (Hahn-Dantona et al., Ann NY Acad. Sci, 878:372-387 (1999). Latent gelatinase B also can be activated by other proteases including cathepsin G, kallikrein, and trypsin or by incubation with p-aminophenylmercuric acetate (APMA).

Gelatinase B cleaves a variety of substrates including collagen type II, human myelin basis protein, insulin, interferon beta, and others. The substrate recognition specificity of gelatinase B has been studied using a phage display library of random hexamers (Kridel et al., (2001), J. Biol. Chem. 276:20572-20578) and has been empirically assessed on a variety of substrates (Descamps, F J et al., (2003) FASEB, 17(8):887-9; Van Den Steen et al., (2002) FASEB, 16: 379-389; and Nelissen et al. (2003) Brain 126: 1371-1381). Gelatinase B typically has a preference for hydrophobic residues at the P1′ position (the position before which cleavage occurs), such as for example Leucine (L). Other amino acid residues that have been recognized as preferentially cleaved by gelatinase B include Phenylalanine (F), Glutamic Acid (E), Tyrosine (Y), and Glutamine (Q). In some cases, protein glycosylation can affect gelatinase B cleavage.

b. Properties of IFN-γ Variants by Removal of Proteolytic Sites

Among the modified IFN-γ polypeptides provided herein are IFN-γ variants modified to remove protease sensitive sites. Particular of modifications of IFN-γ polypeptides provided herein are modifications of IFN-γ that have increased stability by increasing the resistance of the modified IFN-γ to proteolysis. In one example, the modified IFN-γ polypeptides exhibiting increased stability are human IFN-γ polypeptides. The 2D-scanning methodology was used to identify the amino acid changes on hIFN-γ that lead to an increase in stability when challenged either with proteases (blood, intestinal, etc.), blood lysate or serum. Increasing protein stability to proteases (blood, lysate, intestinal serum, etc.), is contemplated herein to provide a longer in vivo half-life for the particular protein molecules and, thus, a reduction in the frequency of necessary administrations to subjects.

Provided herein are IFN-γ molecules that maintain a requisite activity without substantial change and have been rendered less susceptible to digestion by blood and intestinal proteases and therefore display a longer half-life in blood circulation. Such IFN-γ molecules include modified IFN-γ polypeptides with sufficient activity for therapeutic application(s). In one example, activity of modified IFN-γ, such as hIFN-γ, is assessed in an assay by measuring the capacity of the modified IFN-γ to modulate cell proliferation or anti-viral activity when added to the appropriate cells. Prior to the measurement of activity, IFN-γ molecules, for example hIFN-γ molecules, can be challenged with proteases (blood, intestinal, etc.) including conditions mimicking administered conditions, such as serum, blood, saliva, or digestive assays (in vitro assays), and/or administered to a subject such as a mouse or human (in vivo assays) during different incubation or post-injection times. The activity measured, corresponds then to the residual activity following exposure to the proteolytic mixtures. Activity can be compared with an unmodified IFN-γ as a measurement of the effect of the modification on protease stability, for example resistance to proteases, and on the activity. In one example, the unmodified IFN-γ is a wild-type native IFN-γ. In another example, the unmodified IFN-γ is a variant form of IFN-γ that was used as a starting material to introduce further modifications. Modified IFN-γ polypeptides also can be compared with any known IFN-γ polypeptide in any assay known in the art to compare protease sensitivity, and/or any other activity.

Provided herein are modified IFN-γ polypeptides with increased resistance to one or more proteases and thereby, the half-life of the polypeptide. In exemplary embodiments, the half-life of the IFN-γ mutants provided herein is increased by an amount including, but not limited to, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more, when compared to the half-life of the unmodified IFN-γ in either in vivo (human blood, human serum, saliva, digestive fluid, the intestinal tract, etc.), or an in vitro mixture containing one or more proteases. In other embodiments, the half-life of the IFN-γ mutants provided herein is increased by an amount, including but not limited to, at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more, when compared to the half-life of the unmodified IFN-γ in either in vivo (human blood, human serum, saliva, digestive fluid, the intestinal tract, etc.) or an in vitro mixture containing one or more proteases. In an exemplary embodiment, such IFN-γ variants are generated by modifying a human IFN-γ polypeptide. In one such exemplary embodiment, IFN-γ variants are generated by modifying the human IFN-γ polypeptide such as set forth in SEQ ID NOS: 1 and 2.

c. Generation of IFN-γ Variants by Removal of Proteolytic Sites

In an example of generating variants exhibiting increased stability by removal of proteolysis sites, hIFN-γ was modified. The first step in the design of IFN-γ mutants (i.e. hIFN-γ mutants) resistant to proteolysis includes identifying sites vulnerable to proteolysis along the protein sequence. Based on a list of selected blood, intestinal or any other type of proteases considered (Table 2), the complete list of all amino acids and sequences of amino acids in hIFN-γ that can be targeted by those proteases was first determined in silico. The protease targets (amino acids or sequences of amino acids along the IFN-γ polypeptide) are named in silico HITs (is-HITs). Since protease mixtures in the body are quite complex in composition, it can be expected that the majority of the residues in a given protein sequence can be targeted for proteolysis.

The second step in the design of IFN-γ mutants, such as hIFN-γ mutants, that are resistant to proteolysis includes identifying the appropriate replacing amino acids such that if they replaced the natural amino acids in a IFN-γ polypeptide (i.e. hIFN-γ) at is-HITs, the protein (i) becomes resistant to proteolysis; and (ii) elicits a level of activity at least comparable to the wild-type IFN-γ or hIFN-γ polypeptide. The choice of the replacing amino acids must consider the broad target specificity of certain proteases and the need to preserve the physicochemical properties such as hydrophobicity, charge and polarity of essential (e.g., catalytic, binding, etc.) residues in IFN-γ.

“Point Accepted Mutation” (PAM; Dayhoff et al., 1978) can be used as part of the 2D scanning approach. PAM values, originally developed to produce alignments between protein sequences, are available in the form of probability matrices that reflect an evolutionary distance between amino acids. Conservative substitutions of a residue in a reference sequence are those substitutions that are physically and functionally similar to the corresponding reference residues, i.e., that have a similar size, shape, electric charge, and/or chemical properties, including the ability to form covalent or hydrogen bonds and other such interactions. Conservative substitutions show the highest scores fitting with the PAM matrix criteria in the form of accepted point mutations. The PAM250 matrix is used in the frame of 2D-scanning to identify candidate replacing amino acids for the is-HITs in order to generate conservative mutations without affecting protein function. At least the two amino acids with the highest values in PAM250 matrix corresponding to conservative substitutions or accepted point mutations were chosen for replacement at each is-HIT. The replacement of amino acids by cysteine residues is explicitly avoided since this change potentially leads to the formation of intermolecular disulfide bonds.

Briefly, using the algorithm PROTEOL (on-line at infobiogen.fr and at bioinfo.hku.hk/services/analyseq/cgi-bin/proteol_in.pl), a list of residues along the hIFN-γ protein of 146 amino acids (SEQ ID NO: 1), which can be recognized as substrate for proteases (blood, intestinal, etc.) in Table 2 was established. The algorithm generates a proteolytic digestion map based on a list of proteases, the proteolytic specificity of the proteases and the polypeptide amino acid sequence that is entered.

TABLE 2

| | Amino Acid | Protease or chemical |
|---|---|---|
| Abbreviation | Position | Treatment |
| AspN | D | Endoproteinase Asp-N |
| Chymo | (F, W, Y, M, L)˜P | Chymotrypsin |
| Clos | R | Clostripain |
| CnBr | M | Cyanogen Bromide |
| IBzO | W | IodosoBenzoate |
| Myxo | K | Myxobacter |
| NH₂OH | N G | Hydroxylamine |
| pH2.5 | D P | pH 2.5 |
| ProEn | P | Proline Endopeptidase |
| Staph | E | Staphylococcal Protease |
| Tryp | (K, R)˜P | Trypsin |
| TrypK | K˜P | Trypsin (Arg blocked) |
| TrypR | R˜P | Trypsin (Lys blocked) |

Table 2 shows the in silico identification of amino acid positions that are targets for proteolysis using selected proteases and chemical treatment.

Modified IFN-γ polypeptides provided herein exhibit increased resistance to proteolysis by proteases, including those that occur, for example, in body fluids and tissues, such as those that include, but are not limited to, saliva, blood, serum, intestinal, stomach, blood, cell lysates, cells and others. Modifications can include resistance to one or more proteases including, but not limited to, pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, and trypsin, luminal pepsin, microvillar endopeptidase, dipeptidyl peptidase, enteropeptidase, hydrolase, NS3, elastase, factor Xa, Granzyme B, thrombin, trypsin, plasmin, urokinase, tPA and PSA.

Resistance to proteolysis refers to any amount of decreased cleavage of a target amino acid residues of a modified polypeptide by a protease compared to cleavage of an unmodified polypeptide by the same protease under the same conditions. Modified IFN-γ polypeptides provided herein exhibit increased resistance to proteolysis exhibits, for example, at least 1%, 2%, 3%, 4%, 5%, 6%, 7%, 8%, 9%, 10%, . . . 20%, . . . 30%, . . . 40%, . . . 50%, . . . 60%, . . . , 70%, . . . 80%, . . . 90%, 91%, 92%, 93%, 94%, 95%, 96%, 97%, 98%, 99%, or 100% more resistance to proteolysis than an unmodified IFN-γ polypeptide. In some examples, modified IFN-γ polypeptides exhibit 110%, 120%, 130%, 140%, 150%, 200%, 300%, 400%, 500%, or more resistance to proteolysis as compared to the unmodified IFN-γ polypeptide.

Modified polypeptides that are more resistant to gelatinase B are provided. Modifications include those that render polypeptides more resistant to cleavage by a gelatinase B than an unmodified polypeptide that is cleaved by gelatinase B. Non-limiting modifications in an IFN-γ polypeptide that confer increased resistance to gelatinase B can be rationally determined based on the known substrate specificity of gelatinase B (see e.g. Descamps, F J et al., (2003) FASEB, 17(8):887-9). For example, based on the cleavage of gelatinase B substrates, the following amino acids were identified as target amino acids: Phenylalanine (F), Leucine (L), Glutamic Acid (E), Tyrosine (Y), and Glutamine (Q). The precise amino acids cleaved in a polypeptide by gelatinase B can be determined empirically, if needed. Hence, provided herein are polypeptides that are modified at amino acids that are susceptible to cleavage by gelatinase B. Modified polypeptides can be modified at each of residues F, L, E, Y, Q, or a combination thereof, or at amino acids near these regions, thereby rendering the modified peptide more resistant to proteolysis by gelatinase B than the unmodified polypeptide. The modified polypeptides can be at least 0.5%, 1%, 5%, 10%, 15%, 20%, 25%, 30%, 35%, 40%, 45%, 50%, 55%, 60%,65%, 70%, 75%, 80%, 85%, 90%, 95%, 96%, 97%, 98%, 99%, or 100% more resistant to proteolysis by gelatinase B than the unmodified polypeptide. In some instances, the modified polypeptides are 110%, 120%, 130%, 140%, 150%, 200%, 300%, 400%, 500%, or more resistant to cleavage by gelatinase B. In one embodiment, resistance to protease can be empirically tested by any of the assays described herein.

Is-HITS were identified and LEADS created for higher resistance to proteolysis of hIFN-γ. The native amino acids at each of the is-HIT positions and replacing amino acids for increased resistance to proteolysis can include, but are not limited to replacing any of Y, A, L, S, T, I, V, F, Q and M by any of E, D, K, R, N, Q, S and T. Is-HITS and LEADs can include modifications at regions susceptible to proteolysis.

In one example, the position or positions selected for modification include one or more of positions corresponding to amino acid positions Y2, D5, P6, Y7, K9, E10, E12, L14, K15, K16, Y17, F18, D24, D27, N28, L31, F32, L36, W39, D44, R45, F57, L59, F60, F63, K71, E74, K77, E78, D79, K83, F84, K89, K90, K91, R92, D93, D94, F95, E96, K97, L98, Y101, D105, L106, E115, E122, L123, P125,, K128, K131, R132, K133, R134, M137, L138, F139, R142, and R143 of mature human interferon-γ set forth as SEQ ID NO: 1. In one embodiment, an IFN-γ of non-human origin (e.g., bovine, sheep or monkey IFN-γ) is modified. Such alignments and selection of positions can be performed with any IFN-γ polypeptide by aligning it with hIFN-γ and selecting corresponding positions for modification.

In one embodiment, positions corresponding to hIFN-γ are selected (is-HITS) and amino acid replacements are made (LEADs) with increased resistance to proteolysis that include, but are not limited to replacements corresponding to Y2H, Y2I, D5N, D5Q, P6A, P6S, Y7H, Y7I, K9N, K9Q, E10Q, E10H, E10N, E12Q, E12H, E12N, L14I, L14V, K15N, K15Q, K16N, K16Q, Y17H, Y17I, F18I, F18V, D24N, D24Q, D27N, D27Q, N28Q, N28S, L31I, L31V, F32I, F32V, L36I, L36V, W39H, W39S, D44N, D44Q, R45H, R45Q, F57I, F57V, L59I, L59V, F60I, F60V, F63I, F63V, K71N, K71Q, E74Q, E74H, E74N, K77N, K77Q, E78Q, E78H, E78N, D79N, D79Q, K83N, K83Q, F84I, F84V, K89N, K89Q, K90N, K90Q, K91N, K91Q, R92H, R92Q, D93N, D93Q, D94N, D94Q, F95I, F95V, E96Q, E96H, E96N, K97N, K97Q, L98I, L98V, Y101H, Y101I, D105N, D105Q, L106I, L106V, E115Q, E115H, E115N, E122Q, E122H, E122N, L123I, L123V, P125A, P125S, K128N, K128Q, K131N, K131Q, R132H, R132Q, K133N, K133Q, R134H, R134Q, M137I, M137V, L138I, L138V, F139I, F139V, R142H, R142Q, R143H, and R143Q, where the replacements are made compared to the sequence of amino acids set forth in SEQ ID NO: 1. In one embodiment, the mutated IFN-γ polypeptides have an amino acid sequence as represented in any one of SEQ ID NOS: 3-38, 41, 42, 45, 46, 55-60, 63-66, 69, 70 and 77-149. In reference to such mutants, the first amino acid (one-letter abbreviation) corresponds to the amino acid that is replaced, the number corresponds to position in the hIFN-γ polypeptide sequence with reference to SEQ ID NO: 1, and the second amino acid (one-letter abbreviation) corresponds to the amino acid selected that replaces the first amino acid at that position. The IFN-γ polypeptides employed for modification can be any IFN-γ polypeptide, including other mammalian IFN-γ polypeptides. Corresponding positions, as assessed by appropriate alignment, are identified and modified as described herein.

Among the modified IFN-γ polypeptides provided herein are IFN-γ polypeptides that are resistant to one or more than one protease. All mutants are designed so that a single site of proteolysis is destroyed. The particular site for proteolysis can be the target for one (or more) proteases. Thus, modification of the site can render a polypeptide resistant to one or more proteases. In some instances, the site targeted for mutation can be a site that for structural or other reasons can protect the entire molecule from proteolysis irrespective of the particular protease that attacks those particular sites. In one non-limiting example, glycosylation generally protects a molecule from proteolysis. Thus, modification of a target site to create a glycosylation site can in some instances protect the entire molecule from proteolysis.

Provided herein are modified IFN-γ polypeptides that exhibit increased protease resistance to more than one protease as compared to an unmodified IFN-γ polypeptide. In one example, the more than one proteases are selected from among pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, and trypsin, luminal pepsin, microvillar endopeptidase, dipeptidyl peptidase, enteropeptidase, hydrolase, NS3, elastase, factor Xa, Granzyme B, thrombin, trypsin, plasmin, urokinase, tPA and PSA. For example, a modified IFN-γ polypeptide can exhibit protease resistance to 2, 3, 4, 5, 6, 7, 8, 9, 10, or more proteases. These proteases can be present in a protease cocktail in vitro, or can be present in vivo such as for example in serum or in the gastrointestinal tract. In one example, modified IFN-γ polypeptides provided herein exhibit increased protease resistance to a cocktail of three proteases including endoproteinase Glu-C, trypsin, and α-chymotrypsin. Exemplary of such modified polypeptides include F57V, F63I, F63V, K89N, K89Q, K90N, K90Q, E96Q, E96H, E96N, L98I, L98V, D105N, D105Q, L106I, L106V, P125A, P125S, K133N, K133Q, R134H, R134Q, M137V, L138V, F139I, F139V, R142H, R142Q, R143H, and R143Q.

d. Additional IFN-γ Modifications

In particular embodiments, variant IFN-γ polypeptides also can be generated that have one or more amino acids at one or more is-HIT sites that have been replaced by candidate LEAD amino acids. Those mutant proteins carrying one or more mutations at one or more is-HITs, and that display improved protease resistance are called LEADs (one mutation at one is-HIT) and super-LEADs (mutations at more than one is-HIT).

In additional embodiments, mutant molecules that display improved protease resistance, such as LEADs and super-LEADs, can be further modified with additional mutations, such as any of those described herein that confer increased protein stability. For example, protease-resistant LEADs and super-LEADs can be modified to contain mutations including: 1) increasing interactions between helices; 2) increasing interactions between helices of an IFN-γ dimer; and 3) other modification of resistance to proteolysis. In addition, any of the modified polypeptides provided herein can be further modified, such as with any known modification of IFN-γ known in the art as described below.

e. Assessment of IFN-γ Variants with Increased Resistance to Proteolysis

Increased resistance to proteolysis of IFN-γ variants can be assessed by any methods known in the art to assess protein stability, protease sensitivity and resistance and/or IFN-γ activity. In one example, protease resistance is measured by incubating a modified IFN-γ polypeptide with one or more proteases and then assessing residual activity compared to an untreated control. A modified IFN-γ can be compared with an unmodified and/or wild-type native IFN-γ treated under similar conditions to determine if the particular variant retains more activity than the unmodified IFN-γ. Activity can be assessed by any methods known in the art, for example by measuring anti-viral and anti-proliferation activities.

Kinetic studies of protease resistance also can be used to assess a modified IFN-γ polypeptide. For example, a modified IFN-γ polypeptide is incubated with one or more proteases and samples are taken over a series of time-points. At each time point, the proteases are inactivated and the samples are then tested for IFN-γ activity. In one embodiment, the modified polypeptide is at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% more resistant to proteolysis. In some instances, a modified IFN-γ polypeptide is 110%, 120%, 130%, 140%, 150%, 200%, 300%, 400%, 500%, or more resistant to proteolysis.

In one embodiment where the protease is gelatinase B, the modified polypeptide is resistant to cleavage at, for example, hydrophobic amino acids such as L, F, E, Y, and Q, which are preferred sites of cleavage by gelatinase B. In one embodiment, the modified polypeptide is at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% more resistant to proteolysis by gelatinase B at each of residues L, F, E, Y, Q, or a combination thereof. In another example, the modified polypeptide is 110%, 120%, 130%, 140%, 150%, 200%, 300%, 400%, 500%, or more resistant to proteolyisis by gelatinase B. In one embodiment, resistance to protease can be empirically tested by any of the assays described herein.

In one exemplary embodiment, IFN-γ variants, such as hIFN-γ variants, are assessed for protease resistance with a mixture of proteases and proteolytic conditions including pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, and trypsin, luminal pepsin, microvillar endopeptidase, dipeptidyl peptidase, enteropeptidase, hydrolase, NS3, elastase, factor Xa, Granzyme B, thrombin, trypsin, plasmin, urokinase, tPA and PSA. The resistance of the mutant IFN-γ polypeptides compared to wild-type IFN-γ against enzymatic cleavage can be analyzed by mixing IFN-γ polypeptides with proteases. After exposure to proteases, anti-viral and anti-proliferative activities can be assessed. For example, a cytopathic effects (CPE) assay can be used to assess anti-viral activity of modified IFN-γ polypeptides compared to unmodified IFN-γ polypeptides. Specifically, anti-viral activity of IFN-γ can determined by the capacity of the modified IFN-γ polypeptides to protect HeLa cells against EMC (mouse encephalo-myocarditis) virus-induced cytopathic effects. Anti-proliferative activity of IFN-γ can be determined by assessing the capacity of the modified IFN-γ polypeptides to inhibit proliferation of Daudi cells compared to unmodified IFN γ polypeptides.

2. Increased Tolerance to Denaturing Agents (i.e. Thermal Tolerance)

Increased protein stability can include increased protein-half-life under one or more conditions including, but not limited to, increased temperature, particular pH conditions and/or exposure to denaturing ingredients. Among the modified IFN-γ polypeptides, provided herein are IFN-γ polypeptides modified to increase stability in vitro and/or in vivo. Such modifications can include increased interactions between helices, and increased interactions between helices in an IFN-γ dimer.

a. Properties of Stable IFN-γ Variants

In one example, the modified IFN-γ polypeptides that exhibit increased stability are human IFN-γ polypeptides. The 2D-scanning methodology can be used to identify the amino acid changes on IFN-γ that lead to improved stability. For example, as described herein, to increase the stability of hIFN-γ, the first condition is the nature of the amino acids linked to stability of the molecule such as its potential participation in chemical bridges that can participate in stabilization of the molecule. The second premise is typically related to the specific position of those amino acids along the protein structure. Several structural modifications can be made in IFN-γ by amino acid replacements to increase the conformational stability of IFN-γ while either improving or maintaining the requisite activity, such as, but not limited to cell proliferation and/or anti-viral activity. In exemplary embodiments, these modifications result in stability variants with improved (increased) stability, particularly in vivo upon administration as a therapeutic. Improved stability can be assessed for example by improved thermal tolerance, improved stability in the presence of denaturation agents and/or improved stability under particular pH conditions. Such IFN-γ variants exhibit increased protein half-life compared to an unmodified and/or wild-type native IFN-γ. These modifications include, for example, modifications to increase interactions with and between particular helices in the IFN-γ monomer and/or dimer. Generally, because IFN-γ is organized as a dimer, intra-molecule and inter-molecule stability of the polypeptide is a consideration when designing modifications. The dimer is not like a monomer simply duplicated, because each monomer is mixed with each other. Thus, in some instances, a stabilized intra-molecule could lead to difficulties to form a proper dimer.

In one embodiment, the half-life of the IFN-γ variants provided herein is increased by an amount at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more, when compared to the half-life of an unmodified IFN-γ exposed to particular conditions. Such conditions can be for example, but not limited to, thermal conditions between 20° C. and 45° C. In one example, thermal tolerance is assessed at room temperature (i.e., about 25° C.). In another example, thermal tolerance is assessed at a mammalian body temperature, e.g., about 37° C. for humans or when administered to a subject. In other embodiments, the half-life of the IFN-γ variants provided herein is increased by an amount of at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more, when compared to the half-life of an unmodified IFN-γ exposed to particular thermal conditions between 20° C. and 45° C., for example, incubation at room temperature (i.e., about 25° C.) and/or mammalian body temperature (i.e., human body temperature at about 37° C.).

In one example, the position or positions selected for modification include one or more positions corresponding to Y7, V8, A11, L14, Y17, F18, I35, L36, M48, Q51, V53, S54, F55, F57, L59, F60, N62, S72, V73, T75, I76, M80, F84, F95, L98, T99, Q109, and I117 positions of SEQ ID NO: 1. In one embodiment, the modified IFN-γ is a human IFN-γ. In one embodiment, an IFN-γ of non-human origin (e.g., bovine, sheep or monkey IFN-γ) is modified. Such alignments and selection of positions can be performed with any IFN-γ polypeptide by aligning it with hIFN-γ and selecting corresponding positions for modification.

In one embodiment, the modification corresponds to one or more of replacements corresponding to Y7E, Y7D, Y7K, Y7R, Y7N, Y7Q, Y7S, Y7T, V8E, V8D, V8K, V8R, V8N, V8Q, V8S, V8T, A11E, A11D, A11K, A11R, A11N, A11Q, A11S, A11T, L14E, L14D, L14K, L14R, L14N, L14Q, L14S, L14T, Y17E, Y17D, Y17K, Y17R, Y17N, Y17Q, Y17S, Y17T, F18E, F18D, F18K, F18R, F18N, F18Q, F18S, F18T, I35E, I35D, I35K, I35R, I35N, I35Q, I35S, I35T, L36E, L36D, L36K, L36R, L36N, L36Q, L36S, L36T, M48E, M48D, M48K, M48R, M48N, M48Q, M48S, M48T, Q51E, Q51D, Q51K, Q51R, V53E, V53D, V53K, V53R, V53N, V53Q, V53S, V53T, S54E, S54D, S54K, S54R, S54N, S54Q, S54T, F55E, F55D, F55K, F55R, F55N, F55Q, F55S, F55T, F57E, F57D, F57K, F57R, F57N, F57Q, F57S, F57T, L59E, L59D, L59K, L59R, L59N, L59Q, L59S, L59T, F60E, F60D, F60K, F60R, F60N, F60Q, F60S, F60T, N62E, N62D, N62K, N62R, S72E, S72D, S72K, S72R, S72N, S72Q, S72T, V73E, V73D, V73K, V73R, V73N, V73Q, V73S, V73T, T75E, T75D, T75K, T75R, T75N, T75Q, T75S, I76E, I76D, I76K, I76R, I76N, I76Q, I76S, I76T, M80E, M80D, M80K, M80R, M80N, M80Q, M80S, M80T, F84E, F84D, F84K, F84R, F84N, F84Q, F84S, F84T, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, T99S, Q109E, Q109D, Q109K, Q109R, I117E, I117D, I117K, I117R, I117N, I117Q, I117S, and I117T replacements in SEQ ID NO: 1. In one embodiment, the mutant IFN-γ polypeptide is represented by the amino acid sequence of any one of SEQ ID NOS: 150-239, 241-350, 354-361.

i. Creation of Intra-Molecular Stability

IFN-γ polypeptides can be modified such that interactions between helices in a monomer lead to increased intra-molecular stability (intrastability) and, therefore, increased overall protein stability. For example, the pI of IFN-γ of 9.45 indicates a highly charged protein. Increasing polar interactions between helices can reduce the risk of protein denaturation and/or increase the stability. Generally, modified IFN-γ polypeptides retain at least one activity of an unmodified IFN-γ polypeptide. In one embodiment, to improve stability of the IFN-γ, molecular bonds are created between confronted structurally adjacent helices to increase stability of the overall structure. As described above, an IFN-γ homodimer contains two monomer subunits containing six helices each, denoted A1-A6 or B1-B6, that is stabilized by the interactions between helices within and across the subunit interface. Interactions between helices of a monomer can contribute to protein stability of an IFN-γ. In one non-limiting example, one or more of amino acid residues from any of helices 1-6 on a monomer can be modified to increase intra-molecular stability. The amino acids and amino acid positions selected as is-HITs are such that they are oriented in the region where the helices face each other. Solvent accessibility can be considered for selection of is-HITs.

Is-HITS are identified and LEADS created for higher intra-molecular stability of IFN-γ. The native amino acids at each of the is-HIT positions can include, but are not limited to positions corresponding to Y7, A11, L14, Y17, L36, V53, F57, F60, S72, V73, T75, I76, M80, and F84 of human IFN-γ set forth in SEQ ID NO: 1. Such positions can be chosen for example, as positions to stabilize the interaction between helix 1 and helix 4, helix 2 and helix 3, and/or between helix 3 and helix 4 of IFN-γ, where both helices are located in the same monomer (i.e., intra-stability). In one embodiment, modifications stabilize the interaction between helix 1 and helix 4. For example, modifications can be made in helix 1 at amino acid residues corresponding to any one or more of positions Y7, A11, L14 and Y17, and/or in helix 4 at residues corresponding to any one or more of positions S72, T75 and I76. In another embodiment, modifications can be made in an IFN-γ polypeptide to stabilize the interaction between helix 2 and helix 3 by modifying the amino acid residue corresponding to position L36. In another exemplary embodiment, modifications that can stabilize the interaction between helix 3 and helix 4 can include modifications made in helix 3 at amino acid residues corresponding to any one or more of positions V53, F57 and F60, and/or in helix 4 at residues corresponding to any one or more of positions V73, M80, and F84.

In another embodiment, modified IFN-γ polypeptides encompass modifications that increase polar interactions with other amino acid residues and result in increased intra-stability of the polypeptide. The native amino acid at each of the is-HIT positions is replaced by residues increasing polar interaction with other amino acids selected from among, but not limited to, amino acid residues E, D, K, R, N, Q, S and T. In one embodiment, positions corresponding to IFN-γ are selected (is-HITS) and amino acid replacements are made (LEADs), such as to provide increased stability that include, but are not limited to, replacements corresponding to Y7E, Y7D, Y7K, Y7R, Y7N, Y7Q, Y7S, Y7T, A11E, A11D, A11K, A11R, A11N, A11Q, A11S, A11T, L14E, L14D, L14K, L14R, L14N, L14Q, L14S, L14T, Y17E, Y17D, Y17K, Y17R, Y17N, Y17Q, Y17S, Y17T, L36E, L36D, L36K, L36R, L36N, L36Q, L36S, L36T, V53E, V53D, V53K, V53R, V53N, V53Q, V53S, V53T, F57E, F57D, F57K, F57R, F57N, F57Q, F57S, F57T, F60E, F60D, F60K, F60R, F60N, F60Q, F60S, F60T, S72E, S72D, S72K, S72R, S72N, S72Q, S72T, V73E, V73D, V73K, V73R, V73N, V73Q, V73S, V73T, T75E, T75D, T75K, T75R, T75N, T75Q, T75S, I76E, I76D, I76K, I76R, I76N, I76Q, I76S, I76T, M80E, M80D, M80K, M80R, M80N, M80Q, M80S, M80T, F84E, F84D, F84K, F84R, F84N, F84Q, F84S, and F84T of SEQ ID NO: 1. In one example, such amino acid replacements are made in human IFN-γ such as set forth in SEQ ID NOS: 1 and 2.

ii. Increasing Interactions Between Helices of the Dimer

IFN-γ forms a homodimer where helices between monomeric subunits interact to form a stable dimer. Provided herein are methods for generating and producing IFN-γ and variant IFN-γ polypeptides with increased stability due to interactions between structurally adjacent helices in different monomers of the dimeric IFN-γ (i.e., inter-stability).

The increase in stability can include increased protein half-life in vitro and/or increased protein half-life in vivo. As described herein, the methods for designing and generating highly stable, longer lasting proteins, or proteins having a longer half-life include: i) identifying some or all possible target sites on the protein sequence that can participate in the interaction of helices (these sites are referred to herein as is-HITs); ii) identifying appropriate replacing amino acids, specific for each is-HIT, such that upon replacement of one or more of the original (such as native, amino acids at that specific is-HIT), they can be expected to increase the is-HIT's stability while at the same time, maintaining or improving the requisite activity and specificity of the protein (candidate LEADs); and/or iii) systematically introducing the specific replacing amino acids (candidate LEADs) at every specific is-HIT target position to generate a collection containing the corresponding mutant candidate lead molecules. Mutants are generated, produced and phenotypically characterized one-by-one, such as in addressable arrays, such that each mutant molecule contains initially an amino acid replacement at only one is-HIT site. In particular embodiments, such as in subsequent rounds, mutant molecules also can be generated that contain multiple-HIT sites that have been replaced by candidate LEAD amino acids (super-LEADs). In additional embodiments, these candidate LEADs can be further modified with additional mutations that confer protein stability, such as those described herein. For example, the candidate LEADs can be modified to include increasing interactions between helices of the monomer and/or removing protease sensitive sites.

IFN-γ polypeptides can be modified such that interactions between one or more helices across monomers of an IFN-γ homodimer lead to increased inter-molecular stability (interstability) and, therefore, increased overall protein stability. Generally, modified IFN-γ polypeptides retain at least one activity of an unmodified IFN-γ polypeptide. In one embodiment, to improve stability of the IFN-γ, modifications can be made in particular amino acid residues to strengthen molecular bonds between confronted structurally adjacent helices on opposing monomers. As described above, the first monomer of the dimer is designated “A” and the second monomer of the dimer is designated “B,” whereby the six helices of each monomer are designated A1-A6 and B1-B6, respectively. In one non-limiting example, one or more of amino acid residues from helices A1-A6 of one monomer and one or more of amino acid residues from helices B1-B6 of the second monomer can be modified to increase inter-molecular stability. The amino acids and amino acid positions selected as is-HITs are such that they are oriented in the region where a helix of monomer A faces a helix of monomer B. Solvent accessibility can be considered for selection of is-HITs.

Modifications also can be made to an IFN-γ polypeptide to add charges and/or increase polar interactions between helices across monomers of the dimer. In one embodiment, the 2D-scanning process for protein evolution is used to make amino acid modifications to add charges and/or increase polar interactions between helices on opposing monomers, for example between helix A1 and helix B6 (where A helices represents the helices of one IFN-γ in the dimer and B helices represent helices of the second IFN-γ of the dimer), helix A2 and helix B5, helix A3 and helix B5, and/or helix A3and helix B6, thus increasing polypeptide stability. For example, modifications to increase interstability between helix A1 and helix B6 can include modifications of any one of amino acid residues corresponding to positions Y7, V8, A11, L14 and F18 of helix A1 and/or amino acid residue corresponding to position I117 of helix B6. In another example, modifications to increase interstability between helix A3 and helix B5 can include modifications of any one of amino acid residues corresponding to positions M48, Q51, S54, F55 and N62 of helix A3 and/or any one of amino acid residues corresponding to positions F95, L98 and T99 of helix B5. In another example, modifications to increase interstability between helix A3 and helix B6 can include modifications of any one of amino acid residues corresponding to position L59 of helix A3 and/or the amino acid residue corresponding to position Q109 of helix B6. In another example, stability can be increased between helices A2 and B5 by modification of any of amino acid residues corresponding to positions I35 of helix A2 and/or L98 of helix B5.

In one embodiment, positions are chosen to be modified by selected residues that increase polar interactions with other amino acids to improve interaction between helices of the dimer. Modifications include one or more of positions corresponding to Y7, V8, A11, L14, F18, I35, M48, Q51, S54, F55, L59, N62, F95, L98, T99, Q109, and I117 positions of SEQ ID NO: 1. The native amino acid at each of the is-HIT positions is replaced by residues that increase polar interaction with other amino acids such as, for example, E, D, K, R, N, Q, S and T. In one embodiment, positions corresponding to IFN-γ are selected (is-HITS) and amino acid replacements are made (LEADs) with increased stability that include, but are not limited to replacements corresponding to Y7E, Y7D, Y7K, Y7R, Y7N, Y7Q, Y7S, Y7T, V8E, V8D, V8K, V8R, V8N, V8Q, V8S, V8T, A11E, A11D, A11K, A11R, A11N, A11Q, A11S, A11T, L14E, L14D, L14K, L14R, L14N, L14Q, L14S, L14T, F18E, F18D, F18K, F18R, F18N, F18Q, F18S, F18T, I35E, I35D, I35K, I35R, I35N, I35Q, I35S, I35T, M48E, M48D, M48K, M48R, M48N, M48Q, M48S, M48T, Q51E, Q51D, Q51K, Q51R, S54E, S54D, S54K, S54R, S54N, S54Q, S54T, F55E, F55D, F55K, F55R, F55N, F55Q, F55S, F55T, L59E, L59D, L59K, L59R, L59N, L59Q, L59S, L59T, N62E, N62D, N62K, N62R, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, T99S, Q109E, Q109D, Q109K, Q109R, I117E, I117D, I117K, I117R, I117N, I117Q, I117S, I117T replacements in SEQ ID NO: 1. In one example, such amino acid replacements are made in human IFN-γ such as set forth in SEQ ID NOS: 1 and 2.

iii. Stability in Sites Near Glycosylation Sites of IFN-γ

Provided herein are methods for generating and producing IFN-γ and variant IFN-γ polypeptides with increased stability around glycosylation sites. IFN-γ has 2 potential N-linked glycosylation sites corresponding to amino acid positions N28 and N100 of SEQ ID NO: 1. The extent of glycosylation can contribute to the stability of the protein to protease resistance. For example, N28 is important for the stability of the polypeptide; mutation of amino acids at position N28 of a mature IFN-γ polypeptide to, for example, amino acids A, H, or S, have shown a severe decrease in anti-viral activity and a production loss.

In one example, the two glycosylation sites are modified. In another embodiment, the potential N-linked glycosylation position, N28 and/or N100 is modified and one or more surrounding positions is modified. In another example, one or more surrounding positions is modified but the potential N-linked glycosylation position is not modified. For example, in one embodiment, modifications are selected at positions surrounding one or both of the N-linked glycosylation sites to increase stability of the overall structure of IFN-γ. In one embodiment, the region selected for modification surrounds the positions corresponding to N28 of SEQ ID NO: 1. In another embodiment, the region selected for modification surrounds the positions corresponding to N100 of SEQ ID NO: 1. The amino acids and amino acid positions selected as is-HITs are such that they are in the region near to one or both of the N-linked glycosylation sites.

Provided herein are a modified IFN-γ polypeptide with modification of both N-glycosylation sites (i.e. modification of positions corresponding to N28 and N100 of SEQ ID NO: 1). Exemplary of such modifications is a polypeptide where both N-linked glycosylation positions (i.e., N28 and N100) are modified, wherein the replacing amino acid is alanine as represented by the amino acid sequence of SEQ ID NO: 369. Also, provided herein are modified IFN-γ polypeptides exhibiting increased stability around the N28 glycan site. Such modifications require consideration of the location of the N28 glycan site, which is located in a long loop (15 aa between H1 and H2). This loop is exposed to solvent. Consequently, certain modifications, such as for example modifications that would increase charge at the site, would render the polypeptide more accessible to proteases. Provided herein is a mutant polypeptide modified to add a K at this glycan site followed by a P (i.e. N28K/G29P). This combination will avoid digestion by trypsin and also will rigidify the loop and possibly also decrease the chance of aggregation. Thus, in another embodiment, two or more modifications are made in IFN-γ, such as N28K-G29P (SEQ ID NO: 368).

Provided herein are modified IFN-γ polypeptides exhibiting increased stability around the N100 glycan site. N100 is located at the C-terminus of H5; this area is less flexible than the area where the N28 glycan site is located and the hydrophobic vicinity is already buried. Stability around this site can be achieved by modifying interactions of helix 5 of one monomer, for example, with helix 3 of a second monomer, such as is described above for modifications that alter the inter-stability of the IFN-γ polypeptide. For example, stability in sites near glycosylation sites also can be achieved by making modifications between one or more amino acids of helix A3 and/or one or more amino acids of helix B5 (where A helices represents the helices of one IFN-γ in the dimer and B helices represent helices of the second IFN-γ of the dimer). For example, modifications in one or more amino acids of helix A3 and/or one or more amino acids of helix B5 includes modification of one or more amino acid residues corresponding to positions M48, Q51, S54, E55 and N62 of helix A3 and/or modification of one or more amino acid residues corresponding to positions F95, L98 and T99 of helix B5. Glycosylation sites can contribute to the stability and/or activity of IFN-γ.

Is-HITS are identified and LEADS are created for higher stability of IFN-γ. The native amino acid at each of the is-HIT positions can include, but are not limited to positions corresponding to N28, G29, M48, Q51, S54, F55, N62, F95, L98, T99, and N100 of SEQ ID NO: 1. In one example, residues corresponding to Y, A, L, S, T, I, V, F and M are replaced by either E, D, K, R, N, Q, S or T, and residues corresponding to Q and N are replaced by either E, D, K or R. In one embodiment, positions corresponding to IFN-γ are selected (is-HITS) and one or more amino acid replacements are made (LEADs) with increased stability that include, but are not limited to, replacements corresponding to M48E, M48D, M48K, M48R, M48N, M48Q, M48S, M48T, Q51E, Q51D, Q51K, Q51R, S54E, S54D, S54K, S54R, S54N, S54Q, S54T, F55E, F55D, F55K, F55R, F55N, F55Q, F55S, F55T, N62E, N62D, N62K, N62R, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, and T99S of SEQ ID NO: 1.

In one embodiment, the mutant IFN-γ polypeptide is represented by the amino acid sequence of any one of SEQ ID NOS: 214-225, 234-239, 241-249, 274-277, and 324-346. In one example, such amino acid replacements are made in human IFN-γ such as set forth in SEQ ID NOS: 1 and 2.

b. Assessment of Stability of IFN-γ Variants

Stability of IFN-γ variants can be assessed by any methods known in the art to assess protein stability, denaturation and/or IFN-γ activity. In one example, thermal tolerance is selected to assess the stability of IFN-γ. The kinetics of thermal tolerance are measured, for example, by testing activity at particular temperatures, e.g., between 20° C. and 45° C. In one example, thermal tolerance is assessed at 37° C. Briefly, a modified IFN-γ polypeptide is incubated at the selected temperature; and samples are taken over time-points and assessed for residual activity compared to an untreated control. Typically, modified IFN-γ polypeptides retain at least one activity of an unmodified IFN-γ polypeptide. Such assessment can include, for example, a cell proliferation assay for IFN-γ activity. Thermal tolerance can be assessed based on the ability of a modified IFN-γ polypeptide to maintain activity over time at a particular temperature compared to the ability of an unmodified IFN-γ to maintain activity in similar treatments.

3. Super-LEADs and Additional IFN-γ Modifications

IFN-γ modification also can include combining two or more modifications. For example, two or more LEADs can be added in one molecule. For example, any one or more modifications provided herein to confer increase protein stability to an IFN-γ polypeptide can be combined. Modifications that increase proteolysis resistance can be added to other modifications provided herein or known in the art to increase proteolysis resistance. Modifications that increase stability, such as for example to denaturing conditions or modifications that stabilize the polypeptide around glycosylation sites, can be added to other modifications provided herein or known in the art to increase protein stability. Thus, in one example, modifications that increase stability, such as for example to denaturing conditions or modifications that stabilize the polypeptide around glycosylation sites, can be added to modifications provided herein or known in the art to increase proteolysis resistance. Modifications that increase protease resistance and/or stability also can be added to modifications to IFN-γ that alter other functionalities including activity, receptor interactions, modifications that affect post-translation protein modifications and any other known modifications in the art.

A number of techniques are known for adding more than one modification to the same polypeptide including, but not limited to, 3D-scanning, additive directional mutagenesis and oligonucleotide-mediated mutagenesis.

In one example, Additive Directional Mutagenesis (ADM) can be used to assemble on a single mutant protein multiple mutations present on the individual LEAD molecules, so as to generate super-LEAD mutant proteins (see co-pending U.S. application Ser. No. 10/658,355, U.S. Published Application No. US-2004-0132977-A1 and published PCT applications WO 2004/022747 and WO 2004/022593). ADM is a repetitive, multi-step process where, at each step after the creation of the first LEAD (i.e., modified) protein, a new LEAD mutation is added onto the previous LEAD protein to create successive super-LEAD proteins. For example, a population of sets of nucleic acid molecules encoding a collection of new super-LEAD molecules is generated, tested and phenotypically characterized individually in addressable arrays. Super-LEAD molecules are such that each molecule contains a variable number and type of modifications. Those molecules displaying further improved fitness for the particular feature being evolved, are referred to as super-LEADs. Super-LEADs can be generated by other methods known to those of skill in the art and tested by the high throughput methods herein. For purposes herein a super-LEAD typically has activity with respect to the function or activity of interest that differs from the improved activity of a LEAD by a desired amount, such as at least 10%, 20%, 30%, 40%, 50%, 60%, 70%, 80%, 90%, 100%, 150%, 200% or more from at least one of the LEAD mutants from which it is derived. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than at least one of the LEAD molecules from which it is derived. As with LEADs, the change in the activity for super-LEADs is dependent upon the activity that is being evolved. The desired alteration, which can be either an increase or a reduction in activity, depend upon the function or property of interest.

Another method that can be employed to generate two or more mutations in the same polypeptide is using oligonucleotide-mediated mutagenesis referred to as “multi-overlapped primer extensions” (see co-pending U.S. application Ser. No. 10/658,355, U.S. Publication No. US-2004-0132977-A1 and published PCT applications WO 2004/022747 and WO 2004/022593). This method can be used for the rational preparation of LEADs to form super-LEADS. This method allows the simultaneous introduction of several mutations throughout a small protein or protein-region of known sequence. Overlapping oligonucleotides of typically around 70 bases in length (since longer oligonucleotides lead to increased error) are designed from the DNA sequence (gene) encoding the LEAD proteins in such a way that they overlap with each other on a region of typically around 20 bases. Although typically about 70 bases are used to create the overlapping oligonucleotides, the length of additional overlapping oligonucleotides for use can range from about 30 bases up to about 100 bases. Likewise, although typically the overlapping region of the overlapping oligonucleotides is about 20 bases, the length of other overlapping regions for use herein can range from about 5 bases up to about 40 bases. These overlapping oligonucleotides (including or excluding point mutations) act as template and primers in a first step of PCR (using a proofreading polymerase, such as Pfu DNA polymerase, to avoid unexpected mutations) to create small amounts of full-length gene. The full-length gene resulting from the first PCR is then selectively amplified in a second step of PCR using flanking primers, each one tagged with a restriction site in order to facilitate subsequent cloning. One multi-overlapped extension process yields a full-length, multi-mutated nucleic acid molecule encoding a candidate super-LEAD protein containing multiple mutations therein derived from LEAD mutant proteins.

IFN-γ super-LEAD polypeptides provided herein are multi-mutants, each including mutations that provide increased resistance to proteolysis, mutations that all provide increased thermal tolerance or mutations that result in an increased resistance to proteolysis and increased thermal tolerance.

4. Further Modifications

Any one of the modified IFN-γ polypeptides provided herein can contain one or more further modification. The further modification can be any known modification of an IFN-γ polypeptide, such as a modification that alters the stability, immunogenicity, glycosylation, or other property of an IFN-γ polypeptide. The modification also can be a modification that stabilizes an IFN-γ polypeptide by modification of cysteine residues for the creation of disulfide bonds, or modifications of an IFN-γ polypeptide that creates sites for structural modification of the polypeptide, such as for example, sites for PEGylation.

Exemplary further modifications of an IFN-γ polypeptide that can be combined with any one or more of the modifications provided herein includes modification in one or more of positions corresponding to positions 5, 9, 28, 33, 37, 40, 41, 42, 58, 61, 64-66, 86, 88, 124, 127, 128, 133, and 140 in a polypeptide having a sequence of amino acids set forth in SEQ ID NO:1. For example, the modified interferon-γ polypeptide can have further amino acid modifications corresponding to any one or more of D5N, K9Q, N28S, N28A, N28H, L33I, L33V, K37N, K37Q, K40N, K40Q, E41H, E41N, E41Q, E42Q, E42H, E42N, K58N, K58Q, K61N, K61Q, K64N, K64Q, D65N, D65Q, D66N, D66Q, N86D, N88D, S124P, K128E, K133T and Q140R.

In addition, a further modification of a modified IFN-γ polypeptide provided herein can be a modification in an IFN-γ polypeptide known in the art. Such modifications, include but are not limited to, those described in any one or more of U.S. application Publication No. US 2005/0201982, US 2006/008872US 2003/013840, US 2004/0132977; International Patent Publication No. WO 2004/005341; Sareneva et al. (1995) Biochem J. 308:9; Lunn et al. (1992) Protein Engineering, 5:249; Lunn et al. (1992) Protein Engineering, 5:253. For example, a further modification in an IFN-γ polypeptide can include a modification to optimize glycosylation sites of the polypeptide. Such a modification includes an amino acid modification corresponding to S102T with reference to the amino acid sequence set forth in SEQ ID NO:1. Non-limiting examples of further modification(s) in a modified IFN-γ polypeptide provided herein can be any one or more amino acid modification corresponding to any of Q3C, D5C, P6C, K9C, E12C, N13C, K15S, K15T, K16C, Y17C, F18V, N19C, G21S, G21T, G21N, G21C, H22C, D24C, N28Q, N28C, G29F, G29N, G29Y, G29Q, G29V, G29A, G29M, G29I, G29K, G29R, G29T, G29H, G29C, F32V, G34H, G34D, G34A, G34M, G34N, G34T, G34R, G34S, G34C, K37C, N38C, K40S, K40T, K40C, E41N, E41C, E42C, S43C, K46E, K46D, K46Q, M48N, I51N, F55V, F57V, K58C, F60V, N62C, F63V, K64S, K64T, K64C, D65C, D66N, D66C, Q67C, S68C, Q70N, Q70C, K71C, V73N, E74C, T75C, K77C, E78C, N81C, V82C, K83S, K83T, K83C, F84V, F85N, F85V, N86C, S87C, N88S, N88T, N88C, K89C, K90S, K90T, K90C, D93C, F95V, E96C, K97N, K97C, N100Q, Y101F, Y101N, Y101Q, Y101V, Y101A, Y101M, Y101I, Y101K, Y101G, Y101R, Y101T, Y101H, Y101C, Y101S, S102T, V103H, V103D, V103A, V103M, V103N, V103T, V103R, V103S, V103C, T104C, D105C, L106C, N107C, Q109S, Q109T, H114D, H114P, H114T, H114N, H114L, H114Y, E122N, E122C, A127N, K133N, F139V, and R143N based on the numbering of amino acids in SEQ ID NO:1.

In some examples, the further modification can be structural modifications that do not alter the primary sequence of the polypeptide. Such further modifications include chemical derivitation of the polypeptide (i.e., acetylation or carboxylation), glycosylation, phosphorylation, PEGylation, albumination, or other structural modifications known in the art. In addition, modified polypeptides provided herein also can be further modified using ordinary chemical techniques so as to further improve their resistance to proteolytic degradation, to optimize solubility properties, or to render the polypeptide more suitable as a therapeutic agent. For example, the backbone of the peptide may be cyclized to enhance stability (see Friedler et al. (2000) J. Biol. Chem. 275:23783-23789). Analogs may be used that include residues other than naturally occurring L-amino acids, e.g., D-amino acids or non-naturally occurring synthetic amino acids.

E. Production of IFN-γ Polypeptides

1. Expression Systems

IFN-γ polypeptides (modified and unmodified) can be produced by any methods known in the art for protein production, including the introduction of nucleic acid molecules encoding IFN-γ into a host cell, host animal and expression from nucleic acid molecules encoding IFN-γ in vitro. Expression hosts include E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Expression in eukaryotic hosts can include expression in yeasts such as Saccharomyces cerevisae and Pichia Pastoria, insect cells such as Drosophila cells and lepidopteran cells, plants and plant cells such as tobacco, corn, rice, algae and lemna. Eukaryotic cells for expression also include mammalian cells lines such as Chinese hamster ovary (CHO) cells. Eukaryotic expression hosts also include production in transgenic animals, for example, including production in milk and eggs.

Many expression vectors are available for the expression of IFN-γ. The choice of expression vector is influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vectors in the cells.

a. Prokaryotic Expression

Prokaryotes, especially E. coli, provide a system for producing large amounts of IFN-γ (see, for example, Platis et al. (2003) Protein Exp. Purif. 31(2): 222-30; and Khalizzadeh et al. (2004) J. Ind. Microbiol. Biotechnol. 31(2): 63-69). Transformation of E. coli is a simple and rapid technique well known to those of skill in the art. Expression vectors for E. coli can contain inducible promoters that are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λP_{L} promoter.

IFN-γ can be expressed in the cytoplasmic environment of E. coli. The cytoplasm is a reducing environment and for some molecules, this can result in the formation of insoluble inclusion bodies. Reducing agents such as dithiolthreotol and β-mercaptoethanol and denaturants (e.g., such as guanidine-HCl and urea) can be used to resolubilize the proteins. An alternative approach is the expression of IFN-γ in the periplasmic space of bacteria which provides an oxidizing environment and chaperonin-like and disulfide isomerases leading to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility. Typically, temperatures between 25° C. and 37° C. are used. Mutations also can be used to increase solubility of expressed proteins. Typically, bacteria produce aglycosylated proteins. Thus, if proteins require glycosylation for function, glycosylation can be added in vitro after purification from host cells.

b. Yeast

Yeasts such as Saccharomyces cerevisae, Schizosaccharomyces pombe, Yarrowia lipolytica, Kluyveromyces lactis and Pichia pastoris are useful expression hosts for IFN-γ (see for example, Skoko et al. (2003) Biotechnol. Appl. Biochem. 38(Pt3):257-65). Yeast can be transformed with episomal replicating vectors or by stable chromosomal integration by homologous recombination. Typically, inducible promoters are used to regulate gene expression. Example of such promoters include GAL1, GAL7 and GAL5 and metallothionein promoters such as CUP1. Expression vectors often include a selectable marker such as LEU2, TRP1, HIS3 and URA3 for selection and maintenance of the transformed DNA. Proteins expressed in yeast are often soluble and co-expression with chaperonins, such as Bip and protein disulfide isomerase, can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site (e.g., the Kex-2 protease) can be engineered to remove the fused sequences from the polypeptides as they exit the secretion pathway. Yeast also is capable of glycosylation at Asn-X-Ser/Thr motifs.

c. Insects and Insect Cells

Insects and insect cells, particularly using a baculovirus expression system, are useful for expressing polypeptide such as IFN-γ (see, for example, Muneta et al. (2003) J. Vet. Med. Sci. 65(2):219-23). Insect cells and insect larvae, including expression in the haemolymph, express high levels of protein and are capable of most of the post-translational modifications used by higher eukaryotes. Baculoviruses have a restrictive host range which improves the safety and reduces regulatory concerns of eukaryotic expression. Typically, expression vectors use a promoter such as the polyhedrin promoter of baculovirus for high level expression. Commonly used baculovirus systems include baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express IFN-γ polypeptides. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha-fetoprotein, alpha 1-antitrypsin, beta-globin, myelin basic protein, myosin light chain-2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include, but are not limited to, CHO, Balb/3T3, HeLa, MT2, mouse NS0 (non-secreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42). Also, mammalian cells, such as for example, CHO cells, can be modified to express sialyltransferase, e.g. 1,6-sialyltransferase, e.g. as described in U.S. Pat. No. 5,047,335, in order to provide improved glycosylation of the IFN-γ polypeptide variant.

e. Plants

Transgenic plant cells and plants can be used for the expression of IFN-γ. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce proteins (see, for example, Mayfield et al. (2003) PNAS 100:438-442). Because plants have different glycosylation patterns than mammalian cells, this can influence the choice to produce hIFN-γ in these hosts.

2. Purification

Methods for purification of IFN-γ polypeptides from host cells depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary further the proteins can be extracted and further purified using standard methods in the art.

IFN-γ can be purified using standard protein purification techniques known in the art including but not limited to, SDS-PAGE, size fraction and size exclusion chromatography, ammonium sulfate precipitation and ionic exchange chromatography. Affinity purification techniques also can be used to improve the efficiency and purity of the preparations. For example, antibodies, receptors and other molecules that bind IFN-γ can be used in affinity purification. Expression constructs also can be engineered to add an affinity tag such as a myc epitope, GST fusion or His₆ and affinity purified with myc antibody, glutathione resin and Ni-resin, respectively, to a protein. Purity can be assessed by any method known in the art including gel electrophoresis and staining and spectrophotometric techniques.

3. Fusion Proteins

Fusion proteins containing a targeting agent and a modified IFN-γ protein also are provided. Pharmaceutical compositions containing such fusion proteins formulated for administration by a suitable route are provided. Fusion proteins are formed by linking in any order the modified IFN-γ and an agent, such as an antibody or fragment thereof, growth factor, receptor, ligand and other such agent for directing the mutant protein to a targeted cell or tissue. Linkage can be effected directly or indirectly via a linker. The fusion proteins can be produced recombinantly or chemically by chemical linkage, such as via heterobifunctional agents or thiol linkages or other such linkages. The fusion proteins can contain additional components, such as E. coli maltose binding protein (MBP) that aid in uptake of the protein by cells (see, International PCT application No. WO 01/32711).

Fusion proteins of two or more IFN-γ polypeptides also are provided. For example, fusion proteins are formed by linking in any order a modified IFN-γ polypeptide monomer with another modified IFN-γ polypeptide monomer containing the same or different modification. Fusion proteins also can be formed by linking homo- or hetero-dimers of IFN-γ with another homo- or hetero-dimer that is the same of different. In all instances, linkage can be effected directly or indirectly via a linker. The fusion proteins can be produced recombinantly or chemically by chemical linkage, such as via heterobifunctional agents or thiol linkages or other such linkages, such as for example, via a peptide bond or a peptide linker. For example, as mentioned above, IFN-γ generally exists as a dimeric polypeptide. The polypeptide is normally in homodimeric form (e.g. prepared by association of two identical IFN-γ polypeptide molecules). An IFN-γ polypeptide, however, can be provided in single chain form whereby two IFN-γ polypeptide monomers are linked via a peptide bond or a peptide linker. Thus, an IFN-γ polypeptide in single chain form can include two constituent polypeptides that are different, e.g., to enable asymmetric mutagenesis of the polypeptides. In one example, each monomer can contain a different modification that increases the stability of the IFN-γ polypeptide (i.e. one monomer contains a modification that increases protease resistance and the other contains a modification that increases thermal tolerance). In another example, one monomer can contain one or more modifications such as is described herein, and the other monomeric polypeptide can contain one or more other modifications conferring a desired property, such as for example, modifications conferring reduced immunogenicity, increased receptor binding, or any other desired property. In an additional example, one monomer can contain one or more modifications such as is described herein, and the other monomeric polypeptide can contain one or more other modifications providing a desired structural modification, such as for example, a modification providing a PEGylation site, glycosylation site, or other site for any desired structural modification of the polypeptide.

4. Polypeptide Modification

Modified IFN-γ polypeptides can be prepared as naked polypeptide chains or as a complex. For some applications, it can be desirable to prepare modified IFN-γ in a “naked” form without post-translational or other chemical modifications. Naked polypeptide chains can be prepared in suitable hosts that do not post-translationally modify IFN-γ. Such polypeptides also can be prepared in in vitro systems and using chemical polypeptide synthesis. For other applications, particular modifications can be desired including pegylation, albumination, glycosylation, phosphorylation or other known modifications. Modifications can be made in vitro or, for example, by producing the modified IFN-γ in a suitable host that produced such modifications.

5. Nucleotide Sequences

Nucleic acid molecules encoding modified IFN-γ polypeptides or the fusion protein operationally linked to a promoter, such as an inducible promoter for expression in mammalian cells also are provided. Such promoters include, but are not limited to, CMV and SV40 promoters; adenovirus promoters, such as the E2 gene promoter, which is responsive to the HPV E7 oncoprotein; a PV promoter, such as the PBV p89 promoter that is responsive to the PV E2 protein; and other promoters that are activated by the HIV or PV or oncogenes.

Modified IFN-γ polypeptides provided herein also can be delivered to cells in gene transfer vectors. The transfer vectors can encode additional therapeutic agent(s) for treatment of diseases or disorders, such as treatments for viral infection, inherited disorders and others for which IFN-γ is administered. Transfer vectors encoding modified IFN-γ polypeptides can be used systemically by administering the nucleic acid to a subject. For example, the transfer vector can be a viral vector, such as an adenoviral vector. Vectors encoding IFN-γ also can be incorporated into stem cells and such stem cells administered to a subject, for example, by transplanting or engrafting the stem cells at sites for therapy.

F. Assessing IFN-γ Activities

IFN-γ activities and properties can be assessed in vitro and/or in vivo. Assays for such assessment are known to those of skill in the art and are known to correlate tested activities and results to therapeutic and in vivo activities. In vivo assays include IFN-γ assays in animal models as well as administration to humans. For example, the activity of IFN-γ variants can be assessed in vivo and compared to unmodified and/or wild-type IFN-γ activities. IFN-γ variants also can be tested in vivo to assess an activity or property, such as stability (e.g., half-life) and therapeutic effect.

1. In Vitro Assays

Exemplary in vitro assays include assays to assess polypeptide stability and activity. Stability assays include assays that assess protease resistance or thermal stability or other physical property indicative of stability of the polypeptide in vivo or in vitro. Stability also can be assessed by protein structure and conformational assays known in the art. Assays for activity include measurement of IFN-γ interaction with its receptor and cell-based assays to determine the effect of IFN-γ polypeptides variants on cellular pathways.

IFN-γ polypeptides can be tested for anti-viral activity. In one non-limiting example, confluent cells are trypsinized and plated at a density of 2×10⁴ cells/well in DMEM 5% SVF medium on Day 0. Cells are incubated with IFN-γ and/or an IFN-γ variant for 24 hours at 37° C. prior to challenge with virus. When virus induced CPE is near maximum in untreated cells after an incubation of 16 h, the number of viral particles in each well is determined by RT-PCR quantification of viral mRNA using lysates of infected cells. RNA from cell extracts is purified after a DNAse/proteinase K treatment (Applied Biosystems), and the CPE is evaluated using Uptibleu (Interchim) and MTS (Promega) methods which are based on detecting bio-reductions produced by the metabolic activity of cells in a fluorometric and colorimetric manner, respectively. A standard curve for virus quantification is produced for comparison.

In another example of an anti-viral assay, IFN-γ and IFN-γ polypeptide variants are compared in an assay that measures viral CPE. For example, anti-viral activity of IFN-γ is determined by the capacity of the IFN-γ or variant to protect HeLa cells against virus-induced cytopathic effects. HeLa cells (2×10⁵ cells/ml) are seeded in flat-bottomed 96-well plates containing 100 μl/well of Dulbecco's MEM-GlutamaxI-sodium pyruvate medium supplemented with 5% SVF and 0.2% of gentamicin. Cells are grown at 37° C. in an atmosphere of 5% CO₂ for 24 hours. Two-fold serial dilutions of interferon samples are made with MEM complete media into 96-Deep-Well plates with final concentration ranging from, for example, 6000 to 2.926 pg/ml. The medium is aspirated from each well and 100 μl of interferon dilutions are added to HeLa cells. Each interferon sample dilution are assessed in triplicate. The two last rows of the plates are filled with 100 μl of medium without interferon dilution samples in order to serve as controls for cells with and without virus. After 24 hours of growth, a 1/1000 virus dilution solution is placed in each well except for the cell control row. Plates are returned to the CO₂ incubator for 48 hours. Then, the medium is aspirated and the cells are stained for 1 hour with 100 μl of Metilene Blue staining solution to determine the proportion of intact cells. Plates are washed in a distilled water bath and the cell bound dye is extracted using 100 μl of ethylene-glycol mono-ethyl-ether (Sigma). The absorbance of the dye is measured using an ELISA plate reader (Spectramax). The anti-viral activity of IFN-γ samples (expressed as number of IU/mg of proteins, i.e. specific activity) is determined from the concentration needed for 50% protection of the cells against virus-induced cytopathic effects.

The specific activity of modified IFN-γ polypeptides can be compared to an unmodified IFN-γ polypeptide. In some instances the specific activity is unchanged compared to an unmodified IFN-γ polypeptide. Exemplary of modified IFN-γ polypeptides that exhibit a specific activity that is unchanged compared to an unmodified IFN-γ polypeptide include D5Q, Y7H, and E12N. In other cases, the specific activity is decreased compared to an unmodified IFN-γ polypeptide. Exemplary of modified IFN-γ polypeptides that exhibit a decreased specific activity include D5N, K9N, E10N, E12Q, E12H, L14I, L14V, K15N, K15Q, K16N, F18V, L36I, L36V, E42Q, E42H, E42N, R45H, R45Q, F57I, F57V, K58N, K58Q, L59I, L59V, E74Q, E74H, E74N, K77N, K77Q, E78Q, E78H, E78N, K83N, K83Q, R92H, R92Q, E96Q, K97N, L98V, Y101H, and D105Q. In another example, the specific activity is increased compared to an unmodified IFN-γ polypeptide. Exemplary of modified IFN-γ polypeptides that exhibit an increased specific activity include P6A, P6S, Y7I, K9Q, E10Q, E10H, K16Q, Y17H, Y17I, F18I, L33I, L33V, K37N, K37Q, K40N, K40Q, E41Q, E41H, E41N, F60I, K61N, K61Q, F63I, F63V, K64Q, D65N, D66N, D66Q, K71N, K71Q, D79N, K89N, K89Q, K90N, K90Q, E96H, E96N, K97Q, L98I, Y101I, D105N, L106I, L106V, P125A, P125S, K128N, K128Q, R132H, R132Q, K133N, K133Q, R134H, R134Q, M137V, L138V, F139I, F139V, R142H, R142Q, R143H, and R143Q.

In some examples, the unmodified, wild-type IFN-γ or modified IFN-γ can be exposed to conditions that affect protein stability, such as for example, exposure to proteases, temperature or pH. The exposure can occur in vitro or in vivo. For example, a modified IFN-γ polypeptide can be preincubated with a protease or protease cocktail for increasing amounts of time, followed by quenching of the protease activity such as with EDTA or by the addition of an anti-protease solution (ROCHE). In one example, the protease can be pancreatin (SIGMA, i.e., 2.5% w/w) which is a mixture of digestive enzymes including amylase, trypsin, chymotrypsin, and lipase. In another example, the protease cocktail can be a mixture of proteases including endoproteinase Glu-C, 200 μg/ml; trypsin 400 μg/ml and α-chymotrypsin 400 μg/ml. The protease-treated IFN-γ can then be tested for its anti-viral activity in the assay described above to determine if it exhibits residual biological activity following protease treatment. In another example, the pharmacokinetics of a modified or unmodified IFN-γ polypeptide can be assessed to determine if in vivo conditions affect the biological activity of an IFN-γ polypeptide, such as is described below.

2. Non-Human Animal Models

Non-human animal models are useful tools to assess activity and stability of IFN-γ variants. For example, non-human animals can be used as models for a disease or condition. Non-human animals can be injected with disease and/or phenotype-inducing substances prior to administration of IFN-γ variants to monitor the effects on disease progression. Genetic models also are useful. Animals, such as mice, can be generated which mimic a disease or condition by the overexpression, underexpression or knock-out of one or more genes. Such animals can be generated by transgenic animal production techniques well-known in the art or using naturally-occurring or induced mutant strains. Examples of useful non-human animal models of diseases associated with IFN-γ include, but are not limited to, rat models of hepatic fibrosis (including animals induced by dimethylnitrosamine and carbon tertrachloride), transgenic mice expressing IFN-γ and/or an IFN-γ variant in particular cell types (e.g., pancreatic islet cells), and allergen challenged mice. These non-human animal models can be used to monitor activity of IFN-γ variants compared to a wild type IFN-γ polypeptide.

Animal models can further be used to monitor stability, half-life and clearance of modified IFN-γ polypeptides. For example, such in vivo assays can be used to determine the pharmacokinetics of modified IFN-γ polypeptides following various routes of administration. Such assays can be useful for comparing modified IFN-γ polypeptides and for calculating doses and dose regimens for further non-human animal and human trials. For example, a modified IFN-γ polypeptide can be injected into the tail vein of mice (i.e. intravenous). Blood samples are then taken at time-points after injection (such as minutes, hours and days afterwards) and then the level of the modified IFN-γ polypeptides in bodily samples including, but not limited to, serum or plasma can be monitored at specific time-points for example by ELISA or radioimmunoassay. Similar assays also can be performed following intraperitoneal, intraduodenal, intraportal, intragastric, or per os (i.e. oral administration) of modified IFN-γ polypeptides.

In one example, bioavailability of modified IFN-γ polypeptides can be assessed following intraduodenal administration to determine the stability of IFN-γ polypeptides to conditions in the intestine, such as for example, to gastrointestinal proteases including, but not limited to, chymotrypsin or trypsin, and to pH effects. For example, similar conditions would be expected following oral administration of a polypeptide. In such an example, rats can be administered with 2 mg polypeptide/rat via an intraduodenal catheter. Blood samples can be collected at different times and the level of IFN-γ can be monitored by ELISA.

In another example, modified IFN-γ polypeptides can be assessed following per os administration to determine the stability of IFN-γ polypeptides to conditions encountered upon administration of the polypeptides orally by mouth. For animal studies, such as in rats, the oral application can be done by gavage. Oral gavage is a technique used to deliver drugs directly to the stomach. This is used to mimic human consumption where drugs are often swallowed directly rather than consumed throughout a feeding period. This technique also ensures that a known dose of drug is administered directly. Specialised ball tipped needles or flexible catheters is used. The ball at the end of gavage needle protects the oropharyngeal tissues and makes inadvertent endotracheal passage less likely. Considerations in performing oral gavage are the length and diameter of the needle, and the size of the bulb. Typically, gavage volumes should not exceed 1% of body weight (for example, a 20 gram mouse may have 0.2 ml administered). Generally, for rats a 1 ml volume is acceptable. In some cases, gavage of the test polypeptide can be preceded by injection by gavage of a solution of 3% sodium bicarbonate.

In another example, the pharmacokinetics of a modified or unmodified IFN-γ polypeptide can be assessed to determine if in vivo conditions affect the biological activity of an IFN-γ polypeptide. In vivo conditions that can affect protein stability include temperature (i.e. such as at 37° C.), pH changes, exposure to proteases, etc. For example, unmodified wild-type IFN-γ or modified IFN-γ polypeptides can be administered by any of various routes of administration (i.e., intravenous, intraperitoneal, subcutaneous, per os, intraduodenal, intragastric) of an animal or human. Blood samples can be drawn over time. Serial dilutions of the collected plasma can be tested for an IFN-γ activity, such as anti-viral activity. For example, serial dilutions of the collected plasma can be added to HeLa cells to assess for protection of cytopathic effects following infection with ECMV.

Modified IFN-γ polypeptides can be tested for therapeutic effectiveness using animal models for HCV. In one non-limiting example, an animal model such as a chimpanzee is injected with a modified IFN-γ preparation following injection of HCV. Viral load is monitored over time compared to control animals not injected with IFN-γ and/or animals injected with unmodified IFN-γ. Additional immune responses such as induction of Th2 (e.g., IL-4) or Th1 (e.g., IFN-γ) cytokines can be monitored. To determine the ability of freshly purified CD4 positive cells to express IFN-γ and IL-4 an intracellular cytokine (ICC) staining procedure using an Internal Cellular Cytokine (ICC) kit (BioErgonomics, St. Paul, Minn.) can be performed. According to the manufacturer's recommendation, PBMC are stimulated for 20 hours in T-cell activation medium, stained first by surface anti-CD4 antibodies, fixed, permeated and then stained with intracellular anti-IFN-γ and anti-IL-4 antibodies. Samples are analyzed by flow cytometry and results are presented as percentages of IFN-γ and IL-4 expressing cells in CD4+ T cell subset.

Modified IFN-γ polypeptides can be tested for therapeutic effectiveness using animal models for HIV. In one non-limiting example, an animal model such as a HIV1-challenged SCID mouse model for HIV is injected with a modified IFN-γ preparation following injection of HIV1 virus into the model. CD4+ lymphocytes are monitored over time compared to control animals not injected with IFN-γ and/or animals injected with unmodified IFN-γ. Additional immune responses such as induction of Th2 or Th1 cytokines can be monitored.

Modified IFN-γ polypeptides can be tested for therapeutic effectiveness using animal models for MDV. In one non-limiting example, an animal model such as a chicken model for MDV is injected with a modified IFN-γ preparation following injection of MDV into the model. CD4+ lymphocytes are monitored over time compared to control animals not injected with IFN-γ and/or animals injected with unmodified IFN-γ.

Modified IFN-γ polypeptides can be tested for therapeutic effectiveness using animal models for Coxsackievirus B2. In one non-limiting example, an animal model such as a mouse model for Coxsackievirus B3 is injected with a modified IFN-γ preparation following injection of Coxsackievirus B3 into the model. Viral load is monitored over time compared to control animals not injected with IFN-γ and/or animals injected with unmodified IFN-γ.

Modified IFN-γ polypeptides can be tested for therapeutic effectiveness using animal models for tuberculosis. In one non-limiting example, an animal model such as a BALB/c mouse model is administered (e.g., aerosol or intravenous injection) a modified IFN-γ preparation following injection of M. tuberculosis into the model. Bacterial load, cytokine production (Th1 and Th2), body weight, mucus production and lung lesions are monitored over time compared to control animals not injected with IFN-γ and/or animals injected with an unmodified IFN-γ.

Modified IFN-γ polypeptides can be tested for therapeutic effectiveness using animal models for M. leprae and dosages deduced therefrom. In one non-limiting example, an animal model such as a BALB/c mouse model is administered a modified IFN-γ preparation following injection of M. leprae into the model. Bacterial load and cytokine production (Th1 and Th2) are monitored over time compared to control animals not injected with IFN-γ and/or animals injected with an unmodified IFN-γ.

Modified IFN-γ polypeptides can be tested for therapeutic effectiveness using animal models for M. avium. In one non-limiting example, an animal model such as a BALB/c mouse model is administered a modified IFN-γ preparation following injection of M. avium into the model. Bacterial load and cytokine production (Th1 and Th2) are monitored over time compared to control animals not injected with IFN-γ and/or animals injected with an unmodified IFN-γ.

Modified IFN-γ polypeptides can be tested for therapeutic effectiveness using animal models for aspergillosis. In one non-limiting example, an animal model such as a mouse model is administered a modified IFN-γ preparation following injection of Aspergillus into the model. Lung capacity and other symptoms are monitored over time compared to control animals not injected with IFN-γ and/or animals injected with an unmodified IFN-γ.

Modified IFN-γ polypeptides can be tested for therapeutic effectiveness using animal models for candidiasis. In one non-limiting example, an animal model such as a C57B1/6 mouse model is administered a modified IFN-γ preparation following injection of C. albicans into the model. Presence of the fungus in areas such as the mouth and vagina, for example, are monitored over time compared to control animals not injected with IFN-γ and/or animals injected with an unmodified IFN-γ.

Modified IFN-γ polypeptides can be tested for therapeutic effectiveness using animal models for PCP. In one non-limiting example, an animal model such as a BALB/c mouse model is depleted of CD4+ T cells, and is infected by administration of intratracheal inoculations with P. carinii cysts. PCP also can be induced in rats by administering steroids prior to administration of P. carinii. Following establishment of infection, mice or rats are administered an aerosolized preparation containing modified IFN-γ. Prophylaxis of the modified IFN-γ polypeptides also can measured by administering the polypeptides or nucleic acids provided herein prior to administration of P. carinii. Pneumocystosis and lung capacity are monitored over time compared to control animals not injected with IFN-γ and/or animals injected with an unmodified IFN-γ. Lung tissue can be analyzed by histology and intensity of P. carinii infection scored based on inflammatory cell accumulation and level of cysts present.

Modified IFN-γ polypeptides can be tested for therapeutic effectiveness using animal models for coccidioidomycosis. The DBA/2 strain of mice are resistant to C. immitus infection due to innate cell-mediated (Th1) responses, whereas BALB/c mice are susceptible due to innate humoral (Th2) responses. In one non-limiting example, a susceptible animal model such as a BALB/c mouse model is administered an intranasal instillation of 20 C. immitis arthroconidia suspended in pyrogen-free saline or a systemic infection can be established by injecting the mice intraperitoneally with 400 arthroconidia suspended in pyrogen-free saline. Following establishment of infection, a modified IFN-γ preparation can be administered intranasally, intraperitoneally, intramuscularly, etc. Lungs, livers, and spleens are removed and homogenized and disease progression are monitored by assessing numbers of colony forming units (CFU) of C. immitis on mycobiotic medium over time. The effect of the modified IFN-γ polypeptides can be compared to BALB/c animals not injected with IFN-γ and/or BALB/c animals injected with an unmodified IFN-γ. Responses also can be measured compared to DBA/2 mice as controls.

Modified IFN-γ polypeptides can be tested for therapeutic effectiveness using animal models for histoplasmosis. In one non-limiting example, an animal model such as a BALB/c mouse model is administered H. capsulatum yeasts intranasally (either single exposure to initiate disease or re-exposure for recurrent infections) followed by administration of an aerosolized preparation containing modified IFN-γ. Lung capacity and function, colony forming units in lungs livers and spleens, as well as other symptoms, are monitored over time compared to control animals not injected with IFN-γ and/or animals injected with an unmodified IFN-γ.

Modified IFN-γ polypeptides can be tested for therapeutic effectiveness using animal models for cryptococcal meningitis. In one non-limiting example, an animal model such as a SCID mouse model is administered a modified IFN-γ preparation following intraveneous injection of C. neoformans into the model. Dissemination of fungus into lungs, livers and spleens is monitored over time compared to control animals not injected with IFN-γ and/or animals injected with an unmodified IFN-γ. In another example, mice are infected intratracheally with live yeast cells and the histological changes in the lungs and the number of microorganisms in the lung and brain are compared in mice treated with a modified IFN-γ preparation compared to control animals not injected with IFN-γ and/or animals injected with an unmodified IFN-γ.

Modified IFN-γ polypeptides can be tested for therapeutic effectiveness using animal models for the chronic inflammatory granulomatous reaction caused by P. brasiliensis. In one non-limiting example, an animal model such as a C57B1/6 mouse model is administered a modified IFN-γ preparation following intravenous injection of P. brasiliensis into the model. Dissemination of fungus into lungs, livers and spleens, granuloma formation and nitric oxide formation are monitored over time compared to control animals not injected with IFN-γ and/or animals injected with an unmodified IFN-γ.

Modified IFN-γ polypeptides can be tested for therapeutic effectiveness using animal models for cancers. In one non-limiting example, an animal model such as a BALB/c mouse model for colon cancer is injected with a modified IFN-γ preparation following tumor implantation into the model. Tumor size and metastasis is monitored over time compared to control animals not injected with IFN-γ and/or animals injected with unmodified IFN-γ. Additional immune responses such as induction of cell death and stimulation of natural killer cells can be monitored.

3. Clinical Assays

Many assays are available to assess activity of IFN-γ for clinical use. Such assays can include assessment of receptor binding, receptor activation, protein stability and half-life in vivo and phenotypic assays. Exemplary assays include, but are not limited to, in vitro IFN-γ bioassays suitable for clinical use such as radioreceptor assays and cell proliferation, anti-infection and anti-viral assays. Phenotypic assays and assays to assess the therapeutic effect of IFN-γ treatment include assessment of blood levels of IFN-γ (e.g. measurement of serum IFN-γ prior to administration and time-points following administrations including, after the first administration, immediately after last administration, and time-points in between, correcting for the body mass index (BMI)), phenotypic response to IFN-γ treatment including amelioration of symptoms over time compared to subjects treated with an unmodified and/or wild type IFN-γ or placebo.

G. Formulation/Packaging/Administration

Pharmaceutical compositions containing an optimized cytokine produced using methods described herein, including IFN-γ variant (modified) polypeptides, modified IFN-γ fusion proteins or encoding nucleic acid molecules, can be formulated in any conventional manner by mixing a selected amount of the polypeptide with one or more physiologically acceptable carriers or excipients. Selection of the carrier or excipient is within the skill of the administering profession and can depend upon a number of parameters. These include, for example, the mode of administration (i.e., systemic, oral, nasal, pulmonary, local, topical or any other mode) and disorder treated. The pharmaceutical compositions provided herein can be formulated for single dosage (direct) administration or for dilution or other modification. The concentrations of the compounds in the formulations are effective for delivery of an amount, upon administration, that is effective for the intended treatment. Typically, the compositions are formulated for single dosage administration. To formulate a composition, the weight fraction of a compound or mixture thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

1. Administration of Modified IFN-γ Polypeptides

The polypeptides can be formulated as the sole pharmaceutically active ingredient in the composition or can be combined with other active ingredients. The polypeptides can be targeted for delivery, such as by conjugation to a targeting agent, such as an antibody. Liposomal suspensions, including tissue-targeted liposomes, also can be suitable as pharmaceutically acceptable carriers. These can be prepared according to methods known to those skilled in the art. For example, liposome formulations can be prepared as described in U.S. Pat. No. 4,522,811. Liposomal delivery also can include slow release formulations, including pharmaceutical matrices such as collagen gels and liposomes modified with fibronectin (see, for example, Weiner et al. (1985) J Pharm Sci. 74(9): 922-5).

The active compound is included in the pharmaceutically acceptable carrier in an amount sufficient to exert a therapeutically useful effect in the absence of undesirable side effects on the subject treated. The therapeutically effective concentration can be determined empirically by testing the compounds in known in vitro and in vivo systems, such as the assays provided herein. The active compounds can be administered by any appropriate route, for example, oral, nasal, pulmonary, parenteral, intravenous, intradermal, subcutaneous, or topical, in liquid, semi-liquid or solid form and are formulated in a manner suitable for each route of administration.

The modified IFN-γ and physiologically acceptable salts and solvates can be formulated for administration by inhalation (either through the mouth or the nose), oral, transdermal, pulmonary, parenteral or rectal administration. For administration by inhalation, the modified IFN-γ can be delivered in the form of an aerosol spray presentation from pressurized packs or a nebulizer with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of e.g., gelatin for use in an inhaler or insufflator, can be formulated containing a powder mix of a therapeutic compound and a suitable powder base such as lactose or starch.

For pulmonary administration to the lungs, the modified IFN-γ can be delivered in the form of an aerosol spray presentation from a nebulizer, turbonebulizer, or microprocessor-controlled metered dose oral inhaler with the use of a suitable propellant. Generally, particle size is small, such as in the range of 0.5 to 5 microns. In the case of a pharmaceutical composition formulated for pulmonary administration, detergent surfactants are not typically used. Pulmonary drug delivery is a promising non-invasive method of systemic administration. The lungs represent an attractive route for drug delivery, mainly due to the high surface area for absorption, thin alveolar epithelium, extensive vascularization, lack of hepatic first-pass metabolism, and relatively low metabolic activity.

For oral administration, the pharmaceutical compositions can take the form of, for example, tablets, pills, liquid suspensions, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The active ingredient present in the capsule can be in, for example, liquid or lyophilized form. The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically-acceptable saline, pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations also can contain buffer salts, flavoring, coloring and sweetening agents as appropriate.

Preparations for oral administration can be suitably formulated to give controlled release of the active compound. For buccal administration the compositions can take the form of tablets or lozenges formulated in a conventional manner.

The modified cytokines exhibit increased resistance to proteolysis and half-life in in the gastrointestinal tract. Thus, preparations for oral administration can be suitably formulated without the use of protease inhibitors, such as a Bowman-Birk inhibitor, a conjugated Bowman-Birk inhibitor, aprotinin and camostat. In other examples, the preparations for oral administration can be formulated with the use of protease inhibitors.

The compositions for oral administration can be formulated, for example, as gastro-resistant capsules or tablets. Such gastro-resistant capsules are modified release capsules that are intended to resist the gastric fluid and to release their active ingredient or ingredients in the intestinal fluid. They are prepared by providing hard or soft capsules with a gastro-resistant shell (enteric capsules) or by filling capsules with granules or with particles covered with a gastro-resistant coating.

The enteric coating is typically, although not necessarily, a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating to achieve delivery of the active ingredient to the lower gastrointestinal tract. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers, such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, met acrylic acid, methyl acrylate, ammonium methylacrylate, ethyl acrylate, methyl methacrylate and/or ethyl methacrylate (e.g., those copolymers sold under the trade name EUDRAGIT); vinyl polymers and copolymers, such as polyvinyl pyrrolidone (PVP), polyvinyl acetate, polyvinyl acetate phthalate, vinyl acetate crotonic acid copolymer, and ethylene-vinyl acetate copolymers; and shellac (purified lac). Combinations of different coating materials may also be used to coat a single capsule. Exemplary of such gastro-resistant capsules are hard gelatin capsules (sold by Torpac or Capsugel) size 9, coated with cellulose acetate phthalate (CAP) at 12% in acetone.

The enteric coating provides for controlled release of the active agent, such that drug release can be accomplished at some generally predictable location in the lower intestinal tract below the point at which drug release would occur without the enteric coating. The enteric coating also prevents exposure of the hydrophilic therapeutic agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated capsules can permit optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The coating may, and preferably does, contain a plasticizer to prevent the formation of pores and cracks that would permit the penetration of the gastric fluids. Suitable plasticizers include, but are not limited to, triethyl citrate (CITROFLEX 2), triacetin (glyceryl triacetate), acetyl triethyl citrate (CITROFLEC A2), CARBOWAX 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will typically contain less than about 50% by weight, preferably less than about 30% by weight, and more preferably, about 10% to about 25% by weight, based on the total weight of the coating, of a plasticizer, particularly dibutyl phthalate, polyethylene glycol, triethyl citrate and triacetin. The coating may also contain other coating excipients, such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating may be applied to the capsule or tablet using conventional coating methods and equipment. For example, an enteric coating may be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6^{th} Edition (Media, Pa.: Williams & Wilkins, 1995). The coating thickness, as noted above, must be sufficient to ensure that the oral dosage form remains intact until the desired site of topical delivery in the lower intestinal tract is reached.

The modified IFN-γ polypeptides can be formulated as a depot preparation. Such long-acting formulations can be administered by implantation (for example, subcutaneously or intramuscularly) or by intramuscular injection. Thus, for example, the therapeutic compounds can be formulated with suitable polymeric or hydrophobic materials (for example as an emulsion in an acceptable oil) or ion exchange resins, or as sparingly soluble derivatives, for example, as a sparingly soluble salt.

The modified IFN-γ can be formulated, for example, for parenteral administration by injection (e.g., by bolus injection or continuous infusion). Formulations for injection can be presented in unit dosage form (e.g., in ampoules or in multi-dose containers) with an added preservative. The compositions can take such forms as suspensions, solutions or emulsions in oily or aqueous vehicles and can contain formulatory agents such as suspending, stabilizing and/or dispersing agents. Alternatively, the active ingredient can be in powder-lyophilized form for constitution with a suitable vehicle, e.g., sterile pyrogen free water, before use.

The active agents can be formulated for local or topical application, such as for topical application to the skin (transdermal) and mucous membranes, such as in the eye, in the form of gels, creams, and lotions and for application to the eye or for intracisternal or intraspinal application. Such solutions, particularly those intended for ophthalmic use, can be formulated as 0.01%-10% isotonic solutions and pH about 5-7 with appropriate salts. The compounds can be formulated for topical application (see, for example, U.S. Pat. Nos. 4,044,126, 4,414,209 and 4,364,923, which describe aerosols for delivery of a steroid useful for treatment of inflammatory diseases, particularly asthma each of which is incorporated herein by reference in its entirety).

The concentration of active compound in the drug composition depends on absorption, inactivation and excretion rates of the active compound, the dosage schedule, and amount administered as well as other factors known to those of skill in the art. As described further herein, dosages can be determined empirically using dosages known in the art for administration of unmodified interferon-γ, and comparisons of properties and activities (e.g., stability and activities) of the modified IFN-γ compared to the unmodified and/or native IFN-γ.

The compositions, if desired, can be presented in a package, in a kit or dispenser device, that can contain one or more unit dosage forms containing the active ingredient. The package, for example, contains metal or plastic foil, such as a blister pack. The pack or dispenser device can be accompanied by instructions for administration. The compositions containing the active agents can be packaged as articles of manufacture containing packaging material, an agent provided herein, and a label that indicates the disorder for which the agent is provided.

Among the modified IFN-γ polypeptides provided herein are IFN-γ polypeptides modified to increase stability to conditions amendable to oral delivery. Oral delivery can include administration to the mouth and/or gastrointestinal tract. Such modifications can include increased protein-half life under one or more conditions such as exposure to saliva, exposure to proteases in the gastrointestinal tract, and exposure to particular pH conditions, such as the low pH of the stomach and/or pH conditions in the intestine. Modifications can include resistance to one or more proteases including pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, and trypsin, luminal pepsin, microvillar endopeptidase, dipeptidyl peptidase, enteropeptidase, hydrolase, NS3, elastase, factor Xa, Granzyme B, thrombin, trypsin, plasmin, urokinase, tPA and PSA. Modifications also can include increasing overall stability to potentially denaturing or conformation-altering conditions such as thermal tolerance, and tolerance to mixing and aeration (e.g., chewing).

IFN-γ modified for suitability to oral delivery can be prepared using any of the methods described herein. For example, 2D- and 3D-scanning mutagenesis methods for protein rational evolution (see, co-pending U.S. application Ser. No. 10/658,355 and U.S. Publication No. US-2004-0132977-A1 and published International applications WO 2004/022593 and WO 2004/022747) can be used to prepare modified cytokines. Modification of IFN-γ for suitability for oral delivery can include removal of proteolytic digestion sites in a cytokine and/or increasing the overall stability of the cytokine structure. Such IFN-γ variants exhibit increased protein half-life compared to an unmodified and/or wild-type native cytokine in one or more conditions for oral delivery. For example, a modified IFN-γ can have increased protein half-life and/or bioavailability in the mouth, throat (e.g., through the mucosal lining), the gastrointestinal tract or systemically.

In one embodiment, the half-life of the modified IFN-γ polypeptides provided herein is increased by an amount of at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500% or more, compared to the half-life of a native cytokine exposed to one or more conditions for oral delivery. In other embodiments, the half-life of the modified IFN-γ polypeptides provided herein is increased by an amount of at least 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more, compared to the half-life of native cytokine exposed to one or more conditions for oral delivery.

In one example, half-life of the modified IFN-γ polypeptide is assessed by increased half-life in the presence of one or more proteases such as pepsin, trypsin, chymotrypsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, and trypsin, luminal pepsin, microvillar endopeptidase, dipeptidyl peptidase, enteropeptidase, hydrolase, NS3, elastase, factor Xa, Granzyme B, thrombin, trypsin, plasmin, urokinase, tPA and PSA. The modified cytokine can be mixed with one or more proteases and then assessed for activity and/or protein structure after a suitable reaction time. Assessment of half-life also can include exposure to increased temperature, such as the body temperature of a subject; exposure to gastric juices and/or simulated gastric juices; exposure to particular pH conditions and/or a combination of two or more conditions. Following exposure to one or more conditions, activity and/or assessment of protein structure can be used to assess the half-life of the modified cytokine in comparison to an appropriate control (i.e., an unmodified and/or wildtype cytokine protein).

The modified IFN-γ polypeptides can be formulated for oral administration, such as in tablets, capsules, liquids or other suitable vehicle for oral administration. In some examples, the capsules or tablets are formulated with an enteric coating to be gastro-resistant. Preparation of pharmaceutical compositions containing a modified IFN-γ for oral delivery can include formulating modified IFN-γ polypeptides with oral formulations known in the art and described herein. The compositions as formulated do not require addition of protease inhibitors and/or other ingredients that are necessary for stabilization of unmodified and wild-type IFN-γ polypeptides upon exposure of proteases, pH and other conditions of oral delivery. For example, such compositions exhibit stability in the absence of compounds such as actinonin or epiactinonin and derivatives thereof; Bowman-Birk inhibitor and conjugates thereof; aprotinin and camostat.

Additionally, because modified IFN-γ polypeptides provided herein exhibit increased protein stability, there is more flexibility in the administration of pharmaceutical compositions than their unmodified counterparts. Typically, orally ingested polypeptides are administered in the morning before eating (i.e., before digestive enzymes are activated). The modified polypeptides provided herein exhibit protease resistance to digestive enzymes and can offer the ability to administer pharmaceutical compositions containing a modified IFN-γ polypeptide at other periods during the day and under conditions when digestive enzymes are present and active.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Tablets and capsules also can be coated with an enteric coating. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol or fractionated vegetable oils); and preservatives (e.g., methyl or propyl p hydroxybenzoates or sorbic acid). The preparations also can contain buffer salts, flavoring, coloring and/or sweetening agents as appropriate.

Preparations for oral administration can be formulated to give controlled or sustained release or for release after passage through the stomach or in the small intestine of the active compound. For oral administration the compositions can take the form of tablets, capsules, liquids, lozenges and other forms suitable for oral administration. Formulations suitable for oral administration include lozenges and other formulations that deliver the pharmaceutical composition to the mucosa of the mouth, throat and/or gastrointestinal tract. Lozenges can be formulated with suitable ingredients including excipients for example, anhydrous crystalline maltose and magnesium stearate. As noted, modified polypeptides described herein exhibit resistance to blood or intestinal proteases and can be formulated without additional protease inhibitors or other protective compounds. Preparations for oral administration also can include a modified IFN-γ resistant to proteolysis formulated with one or more additional ingredients that also confer proteases resistance, or confer stability in other conditions, such as particular pH conditions.

2. Administration of Nucleic Acids Encoding Modified IFN-γ Polypeptides (Gene Therapy)

Also provided are compositions of nucleic acid molecules encoding the IFN-γ polypeptides and expression vectors encoding them that are suitable for gene therapy. Rather than deliver the protein, nucleic acid can be administered in vivo, such as systemically or by other route, or ex vivo, such as by removal of cells, including lymphocytes, introduction of the nucleic therein, and reintroduction into the host or a compatible recipient.

IFN-γ polypeptides can be delivered to cells and tissues by expression of nucleic acid molecules. IFN-γ polypeptides can be administered as nucleic acid molecules encoding IFN-γ polypeptides, including ex vivo techniques and direct in vivo expression. Nucleic acids can be delivered to cells and tissues by any method known to those of skill in the art. The isolated nucleic acid sequences can be incorporated into vectors for further manipulation. As used herein, vector (or plasmid) refers to discrete elements that are used to introduce heterologous DNA into cells for either expression or replication thereof. Selection and use of such vehicles are well within the skill of the artisan.

Methods for administering IFN-γ polypeptides by expression of encoding nucleic acid molecules include administration of recombinant vectors. The vector can be designed to remain episomal, such as by inclusion of an origin of replication or can be designed to integrate into a chromosome in the cell. IFN-γ polypeptides also can be used in ex vivo gene expression therapy using non-viral vectors. For example, cells can be engineered to express an IFN-γ polypeptide, such as by integrating an IFN-γ polypeptide encoding-nucleic acid into a genomic location, either operatively linked to regulatory sequences or such that it is placed operatively linked to regulatory sequences in a genomic location. Such cells then can be administered locally or systemically to a subject, such as a patient in need of treatment.

Viral vectors, including, for example adenoviruses, herpes viruses, retroviruses and others designed for gene therapy can be employed. The vectors can remain episomal or can integrate into chromosomes of the treated subject. An IFN-γ polypeptide can be expressed by a virus, which is administered to a subject in need of treatment. Viral vectors suitable for gene therapy include adenovirus, adeno-associated virus, retroviruses, lentiviruses and others noted above. For example, adenovirus expression technology is well-known in the art and adenovirus production and administration methods also are well known. Adenovirus serotypes are available, for example, from the American Type Culture Collection (ATCC, Rockville, Md.). Adenovirus can be used ex vivo, for example, cells are isolated from a patient in need of treatment, and transduced with an IFN-γ polypeptide-expressing adenovirus vector. After a suitable culturing period, the transduced cells are administered to a subject, locally and/or systemically. Alternatively, IFN-γ polypeptide-expressing adenovirus particles are isolated and formulated in a pharmaceutically-acceptable carrier for delivery of a therapeutically effective amount to prevent, treat or ameliorate a disease or condition of a subject. Typically, adenovirus particles are delivered at a dose ranging from 1 particle to 1014 particles per kilogram subject weight, generally between 106 or 108 particles to 1012 particles per kilogram subject weight. In some situations it is desirable to provide a nucleic acid source with an agent that targets cells, such as an antibody specific for a cell surface membrane protein or a target cell, or a ligand for a receptor on a target cell.

The nucleic acid molecules can be introduced into artificial chromosomes and other non-viral vectors. Artificial chromosomes, such as ACES (see, Lindenbaum et al. Nucleic Acids Res. 2004 Dec. 7;32(21):e172) can be engineered to encode and express the isoform. Briefly, mammalian artificial chromosomes (MACs) provide a means to introduce large payloads of genetic information into the cell in an autonomously replicating, non-integrating format. Unique among MACs, the mammalian satellite DNA-based Artificial Chromosome Expression (ACE) can be reproducibly generated de novo in cell lines of different species and readily purified from the host cells' chromosomes. Purified mammalian ACEs can then be re-introduced into a variety of recipient cell lines where they have been stably maintained for extended periods in the absence of selective pressure using an ACE System. Using this approach, specific loading of one or two gene targets has been achieved in LMTK(−) and CHO cells.

Anther method for introducing nucleic acids encoding the modified IFN-γ polypeptides is a two-step gene replacement technique in yeast, starting with a complete adenovirus genome (Ad2; Ketner et al. (1994) Proc. Natl. Acad. Sci. USA 91: 6186-6190) cloned in a Yeast Artificial Chromosome (YAC) and a plasmid containing adenovirus sequences to target a specific region in the YAC clone, an expression cassette for the gene of interest and a positive and negative selectable marker. YACs are of particular interest because they permit incorporation of larger genes. This approach can be used for construction of adenovirus-based vectors bearing nucleic acids encoding any of the described modified IFN-γ polypeptides for gene transfer to mammalian cells or whole animals.

The nucleic acids can be encapsulated in a vehicle, such as a liposome, or introduced into a cell, such as a bacterial cell, particularly an attenuated bacterium or introduced into a viral vector. For example, when liposomes are employed, proteins that bind to a cell surface membrane protein associated with endocytosis can be used for targeting and/or to facilitate uptake, e.g. capsid proteins or fragments thereof tropic for a particular cell type, antibodies for proteins which undergo internalization in cycling, and proteins that target intracellular localization and enhance intracellular half-life.

For ex vivo and in vivo methods, nucleic acid molecules encoding the IFN-γ polypeptide is introduced into cells that are from a suitable donor or the subject to be treated. Cells into which a nucleic acid can be introduced for purposes of therapy include, for example, any desired, available cell type appropriate for the disease or condition to be treated, including but not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof.

For ex vivo treatment, cells from a donor compatible with the subject to be treated or cells from the subject to be treated are removed, the nucleic acid is introduced into these isolated cells and the modified cells are administered to the subject. Treatment includes direct administration, such as, for example, encapsulated within porous membranes, which are implanted into the patient (see, e.g., U.S. Pat. Nos. 4,892,538 and 5,283,187 each of which is herein incorporated by reference in its entirety). Techniques suitable for the transfer of nucleic acid into mammalian cells in vitro include the use of liposomes and cationic lipids (e.g., DOTMA, DOPE and DC-Chol) electroporation, microinjection, cell fusion, DEAE-dextran, and calcium phosphate precipitation methods. Methods of DNA delivery can be used to express IFN-γ polypeptides in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

In vivo expression of an IFN-γ polypeptide can be linked to expression of additional molecules. For example, expression of an IFN-γ polypeptide can be linked with expression of a cytotoxic product such as in an engineered virus or expressed in a cytotoxic virus. Such viruses can be targeted to a particular cell type that is a target for a therapeutic effect. The expressed IFN-γ polypeptide can be used to enhance the cytotoxicity of the virus.

In vivo expression of an IFN-γ polypeptide can include operatively linking an IFN-γ polypeptide encoding nucleic acid molecule to specific regulatory sequences such as a cell-specific or tissue-specific promoter. IFN-γ polypeptides also can be expressed from vectors that specifically infect and/or replicate in target cell types and/or tissues. Inducible promoters can be use to selectively regulate IFN-γ polypeptide expression.

Nucleic acid molecules, as naked nucleic acids or in vectors, artificial chromosomes, liposomes and other vehicles can be administered to the subject by systemic administration, topical, local and other routes of administration. When systemic and in vivo, the nucleic acid molecule or vehicle containing the nucleic acid molecule can be targeted to a cell.

Administration also can be direct, such as by administration of a vector or cells that typically targets a cell or tissue. For example, tumor cells and proliferating cells can be targeted cells for in vivo expression of IFN-γ polypeptides. Cells used for in vivo expression of an IFN-γ polypeptide also include cells autologous to the patient. Such cells can be removed from a patient, nucleic acids for expression of an IFN-γ polypeptide introduced, and then administered to a patient such as by injection or engraftment.

Polynucleotides and expression vectors provided herein can be made by any suitable method. Further provided are nucleic acid vectors comprising nucleic acid molecules as described above, including a nucleic acid molecule comprising a sequence of nucleotides that encodes the polypeptide as set forth in any of SEQ ID NOS: 3-38, 41, 42, 45, 46, 55-60, 63-66, 69, 70, 77-239, 241-350, 354-361, 368 and 369 or a fragment thereof. Further provided are nucleic acid vectors comprising nucleic acid molecules as described above and cells containing these vectors.

H. Therapeutic Treatments

IFN-γ polypeptides have therapeutic activity alone or in combination with other agents. The modified polypeptides provided herein are designed to retain therapeutic activity but to exhibit modified properties, particularly increased stability. Such modified properties, for example, can improve the therapeutic effectiveness of the polypeptides and/or can provide for additional routes of administration, such as oral administration. The modified IFN-γ polypeptides and encoding nucleic acid molecules provided herein can be used for treatment of any condition for which unmodified IFN-γ is employed. This section provides exemplary uses of and administration methods. These described therapies are exemplary and do not limit the applications of IFN-γ.

IFN-γ is used for treatment of many diseases and disorders, such as, but not limited to, infections such as viral, bacterial, fungal and protozoa infections, proliferative disorders such as cancer, malignant osteopetrosis, and arterial conditions, chronic granulomatous disease, idiopathic pulmonary fibrosis, and hyper IgE states.

Patients rendered T cell-deficient by advanced disease due an underlying immunosuppressive therapy or disorder (e.g., HIV, a neoplastic disease, solid organ transplant, etc.) are vulnerable to opportunistic infections that often fail to respond to traditional therapies, but which do respond to immunotherapeutic approaches, such as IFN-γ, that include activation of macrophages and monocytes or enhancement of T cell function (Murray, H W. Clin. Infec. Dis. 19(Suppl 2): S407-S413 (1993)).

The modified IFN-γ polypeptides provided herein can be used in various therapeutic as well as diagnostic methods in which IFN-γ is employed. Such methods include, but are not limited to, methods of treatment of physiological and medical conditions described and listed below. Modified IFN-γ polypeptides provided herein can exhibit improvement in in vivo activities and therapeutic effects compared to wild-type IFN-γ, including lower dosage to achieve the same effect, a more sustained therapeutic effect and other improvements in administration and treatment.

The modified IFN-γ polypeptides described herein exhibit increased protein stability and improved half-life. Thus, modified IFN-γ polypeptides can be used to deliver longer-lasting, more stable therapies. Examples of therapeutic improvements using modified IFN-γ polypeptides include, but are not limited to, lower dosages, fewer and/or less frequent administrations, decreased side effects and increased therapeutic effects.

Treatment of diseases and conditions with modified IFN-γ polypeptides can be effected by any suitable route of administration using suitable formulations as described herein including, but not limited to, injection, pulmonary, oral and transdermal administration. If necessary, a particular dosage and duration and treatment protocol can be empirically determined or extrapolated. For example, exemplary doses of recombinant and native IFN-γ polypeptides can be used as a starting point to determine appropriate dosages. Modified IFN-γ polypeptides that are more stable and have an increased half-life in vivo, can be effective at reduced dosage amounts and or frequencies. For example, because of the improvement in properties such as serum stability, dosages can be lower than comparable amounts of unmodified IFN-γ. Dosages for unmodified IFN-γ can be used as guidance for determining dosages for modified IFN-γ. Factors such as the level of activity and half-life of the modified IFN-γ in comparison to the unmodified IFN-γ can be used in making such determinations. Particular dosages and regimens can be empirically determined.

The modified IFN-γ proteins herein, and nucleic acids encoding modified IFN-γ polypeptides can be used for treatment of hepatitis C virus infections and associated fibrosis. The modified IFN-γ polypeptides herein provide increased protein stability and improved half-life. Thus, modified IFN-γ can be used to deliver longer lasting, more stable anti-viral therapies. Examples of therapeutic improvements using modified IFN-γ polypeptides include, for example, but are not limited to, lower dosages, fewer and/or less frequent administrations, decreased side effects and increased therapeutic effects.

Dosage levels and regimens can be determined based upon known dosages and regimens, and, if necessary can be extrapolated based upon the changes in properties of the modified polypeptides and/or can be determined empirically based on a variety of factors. Such factors include body weight of the individual, general health, age, the activity of the specific compound employed, sex, diet, time of administration, rate of excretion, drug combination, the severity and course of the disease, and the patient's disposition to the disease and the judgment of the treating physician. The active ingredient, the polypeptide, typically is combined with a pharmaceutically effective carrier. The amount of active ingredient that can be combined with the carrier materials to produce a single dosage form or multi-dosage form can vary depending upon the host treated and the particular mode of administration.

Upon improvement of a patient's condition, a maintenance dose of a compound or compositions can be administered, if necessary; and the dosage, the dosage form, or frequency of administration, or a combination thereof can be modified. In some cases, a subject can require intermittent treatment on a long-term basis upon any recurrence of disease symptoms or based upon scheduled dosages.

The following are some exemplary conditions for which IFN-γ has been used as a treatment agent alone or in combination with other agents.

1. Infections

IFN-γ boosts cells and families of cells and activities of the immune system. IFN-γ has been used for the treatment of infections including bacterial, viral, fungal and parasitic protozoan infections and can be used for treatment of immunocompromised subjects who are especially susceptible to viral, bacterial, fungal or protozoa infections, or a combination thereof, due to the diminished capacity of the immune system to fight off infections.

Treatments of infections include treatment of viral infections such as HCV and HIV; bacterial infections including tuberculosis, leprosy and non-tuberculosis mycobacteria infections; fungal infections including aspergillosis, and candidiasis; and parasitic protozoan infections including toxoplasmosis.

a. Viral Infections

Free radical molecular species play a role in various viral diseases. For example, pro-inflammatory cytokines, such as IFN-γ, induce the inorganic radical nitric oxide (NO) production from macrophages, and nitric oxide has been implicated in host defense mechanisms of viral infections (Akaike et al. Proc. Soc. Exp. Biol. Med. 217(1): 64-73 (1998)).

i. Hepatitis C Virus

IFN-γ therapy has been used for treatment of fibrotic liver diseases, such as chronic hepatitis C viral (HCV) infection, which are characterized by fibrosis of the liver. Inadequate Th1 immunity, as well as weak hepatitis C virus- (HCV-) specific T-cell responses at sites of inflammation are associated with failure to clear the virus and modulate the chronic nature of the disease. Several lines of evidence indicate that HCV is not directly cytopathic for infected host cells and that the immune response plays an important role in the pathogenesis of liver damage. Specifically, insufficient T helper and cytotoxic T lymphocyte (CTL) responses and lower production of cytokines, such as IFN-γ, are the main causes for the lack of Th1 responses during infection. IFN-γ has been shown to inhibit HCV virion production (via inhibiting viral RNA and protein synthesis), enhance immune lysis of HCV-infected cells, inhibit hepatic fibrosis (liver disease) by affecting TGF-β and inhibit HCV-induced carcinogenesis (Cecere et al. Panminerva Med. 46(3): 171-187 (2004); Missale et al. Dig. Liver Dis. 36(11): 703-711 (2004)). IFN-γ has been detected in livers of chimpanzees that cleared or controlled HCV infection (Kakimi K., Hum. Cell 16(4): 191-197 (2003)). Immunotherapy with IFN-γ has been used to treat and control chronic hepatitis C infections. Thus, the modified IFN-γ polypeptides herein, and nucleic acids encoding modified IFN-γ polypeptides can be used in treatment of liver disease, such as in HCV therapies.

ii. Human Immunodeficiency Virus (HIV)

Defective immunological function of cells of the macrophage lineage contributes to the pathogenesis of HIV-1 infection, and progression of HIV is associated with a switch from a Th1 to a Th2 profile. In vitro, IFN-γ has restorative properties for diseases that are categorized as exhibiting a Th2 profile, and has been shown to activate T cells and stimulate nitric oxide production from macrophages. In the case of HIV, functional restoration and protective properties of the immune system and anti-viral activity have been associated with administration of IFN-γ (Brodie et al. J. Immunol. 153(12): 5790-5801 (1994); Kedzierska et al. J. Clin. Virol. 26(2): 247-263 (2003)).

Gene therapy protocols have been used to transfer of IFN-γ genes. For example, myeloid U937 cells transfected with expression vectors containing the IFN-γ genes under the control of the long terminal repeat (LTR) sequences of HIV1 were shown to be strongly resistant against in vitro and in vivo (i.e., HIV1-challenged SCID mice) HIV1 infection. Cellular resistance is correlated with induction of IFN-γ (Leissner et al. Ann. Biol. Clin. (Paris) 56(2): 167-173 (1998)).

Administration of recombinant IFN-γ has been used to treat infections in immunocompromised patients, including patients with HIV or AIDS. For example, administration of recombinant IFN-γ to AIDS patients having acute cryptococcal meningitis infections has been shown to decrease viral load (Clemons et al. Antimicrobial Agents and Chemotherapy 45(3): 686-689 (2001)). IFN-γ therapy has been used in treatments of fungal pathogen infections including, but not limited to, invasive aspergillosis and candidemia which are opportunistic infections in AIDS patients (Youza et al. An. Med. Interna. 17(2): 86-87 (2000); Jones-Carson et al. Nat. Med. 1(6): 552-557 (1005); Kullberg et al. J. Infect. Dis. 168(2): 436-443 (1993)).

The modified IFN-γ proteins herein, and nucleic acids encoding modified IFN-γ polypeptides can be used to control HIV infection and progression as well as treat opportunistic infections that are common in HIV patients.

iii. Marek's Disease Virus

IFN-γ and nitric oxide (NO) have been shown to be important for controlling viral replication during the lytic phase of infection and for prevention of reactivation of Marek's disease virus replication in latently-infected and transformed cells. Nitric oxide induced by IFN-γ has been shown to inhibit replication of MDV in vitro and in vivo as has been shown for herpes simplex virus (HSV; Schat and Xing, Dev. Comp. Immunol. 24(2-3): 201-221 (2000); Xing and Schat, J. Virol. 74(8): 3605-3612 (2000)). Thus, the modified IFN-γ polypeptides provided herein can be used to treat Marek's disease virus.

iv. Coxsackievirus B3

Administration of IFN-γ has a therapeutic effect in treatment of Coxsackievirus B3 (a member of the picornavirus family), one of the causes of virus-induced acute or chronic heart disease (Henke et al. Expert Rev. Vaccines 2(6): 805-815 (2003)). Thus, the modified IFN-γ proteins herein, and nucleic acids encoding modified IFN-γ polypeptides can be used to treat viruses such as Coxsackievirus B3.

b. Bacterial Infections

i. Tuberculosis

The important role of Th1 responses (e.g., IFN-γ) in resistance to M. tuberculosis comes from children defective in IFN-γ production and from animal (mouse) models. Inhibition or killing of M. tuberculosis is induced in murine macrophages after exposure to IFN-γ. IFN-γ has been shown to prevent and/or treat tubercular infections through enhancement of T cell function and macrophage activation and can be administered to treat pulmonary tuberculosis, including multi-drug resistant forms to decrease lung fibrosis (Koh et al. J. Korean Med Sci. 19: 167-71 (2004); Toossi, Z. Cytokines Cell. Mol. Ther. 4(2): 105-112 (1998); and Rook et al. Eur. Respir. J. 17: 537-557 (2001)). Thus, the modified IFN-γ proteins herein, and nucleic acids encoding modified IFN-γ polypeptides can be used in M. tuberculosis therapies.

ii. Leprosy

Aerosol administration of IFN-γ activates alveolar macrophages; and monocytes from IFN-γ-treated leprotic patients all respond with an activated phenotype. IFN-γ has been administered to treat patients with leprosy (Bermudez and Kaplan, Trends Microbiol. 3(1): 22-7 (1995); Murray, H W. Intensive care Med. 22(Supl 4): S456-461 (1996)). Thus, the modified IFN-γ proteins herein, and nucleic acids encoding modified IFN-γ polypeptides can be used in M. leprae therapies.

iii. Non-Tuberculosis Mycobacterium

The most frequent bacterial infections in patients infected with HIV and suffering from AIDS are non-tubercolous mycobacterial infections (Payen et al. Rev. Mal. Respir. 14(Suppl 5): 142-151 (1997)). Mycobacterium avium complex (MAC) infects most, if not all, HIV-positive patients.

In vivo, a correlation has been found between resistance of mice to M. avium infection and levels of IFN-γ expression (Appelberg, R. Immunobiology 191(4-5): 520-525 (1994). Non-tuberculosis mycobacteria (NTM) infections have been treated by administering IFN-γ polypeptides and IFN-γ polypeptides also have been used as a prophylactic measure (Hallstrand et al. (2004) Eur. Respir. J. 24: 367-70; Famularo et al, Ann Ital Med Int. 9(4):249-54 (1994)). Administration of IFN-γ by ultrasonic nebulization, for example, results in sustained clearance of the bacteria from the airways and stabilization of lung function. Thus, the modified IFN-γ proteins herein, and nucleic acids encoding modified IFN-γ polypeptides can be used in NTM therapies, such as prophylaxis or treatment of disseminated MAC infections.

c. Fungal Infections

Opportunistic fungal pathogens are most detrimental to immunosuppressive subjects such as those taking immunosuppressive regimens prior to, and subsequent to, solid organ or bone marrow transplant, cancer patients, and immunosuppressed HIV patients. Fungal pathogens for which IFN-γ is an effective therapeutic agent include, but are not limited to, Aspergillus species, Candida species, Coccidioides immitis, Histoplasma capsulatum, Pneumocystis carinii, and Cryptococcus neoformans.

i. Aspergillosis

Aspergillus spores release factors that can suppress the synthesis of pro-inflammatory Th1 cytokines, and Th2 reactivity has been shown to lead to disease progression. IFN-γ has been shown to reduce the frequency of Aspergillus species infections in subjects having chronic granulomatous disease and HIV (Youza et al. An. Med. Interna. 17(2): 86-87 (2000); Jones-Carson et al. Nat. Med. 1(6): 552-557 (1005); Kullberg et al. J. Infect. Dis. 168(2): 436-443 (1993)). IFN-γ also has been shown to prevent the deleterious effects of agents such as dexamethasone on anti-fungal activity of human monoctyes that act against Aspergillus (Roilides et al. J. Med. Vet. Mycol. 34(1): 63-69 (1996)). Thus, the modified IFN-γ proteins herein, and nucleic acids encoding modified IFN-γ polypeptides can be used in Aspergillus therapies.

ii. Candidiasis

Neutrophils and monocytes are involved in the non-specific clearance of yeasts such as Candida albicans and Th1 responses are protective via release or administration of IFN-γ. IFN-γ has been effective as a modulator of resistance against C. albicans in vitro and against disseminated candidiasis in an in vivo mouse model (Altamura et al. Curr. Drug Targets Immune Endocr. Metabol. Disord. 1(3): 189-197 (2001); Kullbert et al. Biotherapy 7(3-4): 195-210 (1994)). In one instance of an HIV-infected patient with oropharyngeal candidiasis, subcutaneous IFN-γ injection was efficacious in improving symptoms and signs of oropharyngeal candidiasis (Bodasing et al. J. Antimicrob. Chemother. 50: 765-766 (2002)). IFN-γ induces macrophage nitric oxide production and regulates macrophage function in vivo to enhance resistance to mucosal candidiasis, and activates polymorphonuclear leukocytes to induce host resistance to acute disseminated C. albicans infections in mice (Jones-Carson et al. Nat. Med. 1(6): 552-557 (1005); Kullberg et al. J. Infect. Dis. 168(2): 436-443 (1993)).

While IFN-γ has been shown to have a therapeutic effect on mucosal and non-mucosal (e.g., gastrointestinal) candidiasis, it appears that overproduction of IFN-γ may be involved in the acute pathology of fungal septic shock (Cenci et al. J. Immunol. 161: 3543-3550 (1998); Lavigne et al. J. Immunol. 160: 284-292 (1998)). Thus, there is a need for modified IFN-γ polypeptides that can be given at lower doses.

The modified IFN-γ polypeptides provided herein, and nucleic acids encoding modified IFN-γ polypeptides can be used in candidiasis therapies, and can provide increased protein stability and improved half-life. Thus, modified IFN-γ can be used to deliver longer lasting, more stable anti-fungal therapies at lower dosages.

iii. Pneumocystis jiroveci Pneumonia

Pneumocystis jiroveci (formerly known as Pneumocystis carinii) is a fungus that is the causative agent of Pneumocystis carinii pneumonia (PCP). PCP in AIDS patients is due, in part, to impaired local release of IFN-γ from lung lymphocytes and subsequent failure to activate macrophages. Tissue production of IFN-γ has a protective role in pneumonia and reduces the intensity of P. carinii infection in vivo (Steele et al. Infection and Immunity 70(9): 5208-5215 (2002); Beck et al. Infect. Immun. 59(11): 3859-3862 (1991)). IFN-γ also has a prophylactic effect in vivo (Shear et al. J. Acquir. Immune Defic. Syndr. 3(10): 943-948 (1990)). IFN-γ-mediated resistance is, in part, due to inhibition of P. carinii attachment to alveolar epithelial cells caused by IFN-γ-induced decreases in alveolar integrin expression (Pottratz and Weir. Eur. J. Clin. Invest. 27(1): 17-22 (1997)). Thus, the modified IFN-γ proteins herein, and nucleic acids encoding modified IFN-γ polypeptides can be used to treat patients having pneumonia caused by P. carinii infection.

iv. Coccidioidomycosis

Th2 responses have been shown to exacerbate disease progression caused by Coccidioides immitis infections whereas Th1 responses mediated by IFN-γ are protective and play an important role in resistance to C. immitis (Magee and Cox. Infect. Immun. 63(9): 3514-3519 (1995)). For example, critically ill human patients infected with disseminated coccidioidomycosis, for which conventional anti-fungal therapy (e.g., amphotericin B) was not successful, receiving IFN-γ therapy exhibited improvement in symptoms and discharge from the hospital (Kuberski et al. Chin. Infect. Dis. 38(6): 910-912 (2004)). Thus, the modified IFN-γ proteins herein, and nucleic acids encoding modified IFN-γ polypeptides can be used in coccidioidomycosis therapy.

v. Histoplasmosis

In vitro studies have demonstrated that IFN-γ activates murine peritoneal macrophages to inhibit intracellular growth of H. capsulatum as a result of increased nitric oxide levels. IFN-γ is an important cytokine in the protective immune response in mice against H. capsulatum. Injection of IFN-γ in vivo pre-infection and/or post-infection prolongs survival of infected subjects and causes a significant reduction in fungal burden in the spleen over untreated controls. Thus, IFN-γ has been used prophylactically and therapeutically in initial treatment of histoplasmosis and in re-current infections (Clemons et al. Microbes Infect. 3(1): 3-10 (2001)). Thus, the modified IFN-γ proteins herein, and nucleic acids encoding modified IFN-γ polypeptides can be used in histoplasmosis therapy.

vi. Cryptococcal Meningitis

Protective responses in in vivo murine cryptococcosis models have been demonstrated to be due to the effect of IFN-γ. Studies have shown that IFN-γ, administered prophylactically and therapeutically, or only therapeutically, is efficacious in treating C. neoformans infections in the brain, lungs and liver (Biondo et al. Infection and Immunity 71(9): 5412-5417 (2003); Clemons et al. Antimicrob Agents Chemother. 45(3): 686-689 (2001)). Thus, the modified IFN-γ proteins herein, and nucleic acids encoding modified IFN-γ polypeptides can be used in therapy of cryptococcal meningitis.

vii. Paracoccidioides brasiliensis

Activated macrophages, neutrophils and natural killer cells are able to kill or inhibit the growth of P. brasiliensis in vitro; and in vivo macrophages and lymphocytes control the disease through TNF, IFN-γ and nitric oxide production. The absence of IFN-γ leads to incipient granulomas which are unable to control spread of the fungus (Suoto et al. Am. J. Pathology 156(5): 1811-1820 (2000); Suoto et al. Am. J. Pathology 163(2): 583-590 (2003); Calvi et al. Microbes Infect. 5(2): 107-113 (2003)). Thus, the modified IFN-γ proteins herein, and nucleic acids encoding modified IFN-γ polypeptides can be used in therapy of the chronic inflammatory granulomatous reaction caused by P. brasiliensis.

d. Parasitic Protozoa

Protozoan parasites are characterized as intracellular infections that take place primarily in macrophages. IFN-γ provides one of the major signals for activation of macrophages, which are required for killing parasitic protozoans. Intracellular growth of Toxoplasma gondii in human fibroblasts in vitro is inhibited by IFN-γ, and IFN-γ has been recognized as playing an important role in protection against Toxoplasma gondii in vivo (Subauste and Remington. Eur. J. Clin. Microbiol. Infect. Dis. 10(2): 58-67 (1991); and Brown et al. Adv. Exp. Med. Biol. 294: 425-435 (1991)). Thus, the modified IFN-γ proteins herein, and nucleic acids encoding modified IFN-γ polypeptides can be used in therapy of toxoplasmosis.

2. Proliferative Disorders

A number of proliferative diseases and disorders are targets for IFN-γ therapy. Exemplary proliferative diseases and disorders include, for example, cancers such as T cell lymphoma, melanomas, sarcomas, advanced renal cell carcinoma, primary brain tumors, ovarian carcinoma, prostate cancer, pancreatic cancer, basal cell carcinoma; bone disorders such as malignant osteopetrosis; atherosclerosis, neointimal hyperplasia, restenosis. The modified IFN-γ proteins herein, and nucleic acids encoding modified IFN-γ polypeptides can be used in therapies for proliferative diseases and disorders. The modified IFN-γ polypeptides provided herein increase protein stability and improved half-life and, thus, can be used to deliver longer lasting, more stable therapies. Examples of therapeutic improvements using modified IFN-γ polypeptides include, for example, but are not limited to, lower dosages, fewer and/or less frequent administrations, decreased side effects and increased therapeutic effects.

a. Cancer Therapy

IFN-γ therapy can be used to treat a wide variety of cancers, including but not limited to, T cell lymphoma, melanomas, sarcomas, advanced renal cell carcinoma, primary brain tumors, ovarian carcinoma, prostate cancer, pancreatic cancer, basal cell carcinoma. Treatments include, but are not limited to, intraperitoneal and intravenous administration of IFN-γ. Cancer treatments include reduction of metastasis as well as treatment at tumor sites. Modes of administration include, but are not limited to, IFN-γ protein injection or administration of the nucleic acid molecules encoding modified IFN-γ polypeptides provided herein and also can include additional treatments administered similarly or through other routes of administration, including oral administration and inhalation, stem cell engraftment at tumor sites, administration of an adenovirus vector encoding an IFN-γ systemically and/or at the tumor site. IFN-γ also can be expressed in stem cells and stem cell engrafted at the tumor site used for targeted therapy. Patients rendered T cell-deficient by advanced disease due an underlying neoplastic disorder are vulnerable to opportunistic infections that often fail to respond to traditional therapies, but which do respond to immunotherapeutic approaches such as IFN-γ, that activate macrophages and monocytes or enhance T cell function (Murray, H W. Clin. Infec. Dis. 19(Suppl 2): S407-S413 (1993)).

Administration of IFN-γ to cancer patients has been shown to prevent tumor development (Ikeda et al. Cytokine & Growth Factor Reviews 13: 95-109 (2002)). For example, treatment of epithelial ovarian cancer with recombinant IFN-γ can decrease cell proliferation. In addition, IFN-γ treatment can result in cancer cell apoptosis, anti-proliferation and inhibition of angiogenesis. IFN-γ also can promote host responses to tumors (see, for example, Wall et al. Clin. Cancer Res. 9:2487-96 (2003); Melichar et al. J. Trans Med. 1:5 (2003); Ikeda et al. Cytokine Growth Factor Reviews 13:95-109 (2002); Mocellin et al. (2001) J. Immunotherapy 24:392-407; and Borden et al. (2000) Cancer Biology 10: 125-44). Cancers that can be treated with IFN-γ include, but are not limited to, T cell lymphoma, melanomas, sarcomas, advanced renal cell carcinoma, primary brain tumors, ovarian carcinoma, prostate cancer, pancreatic cancer and basal cell carcinoma. Thus, the modified IFN-γ proteins herein, and nucleic acids encoding modified IFN-γ polypeptides can be used in cancer therapies.

b. Malignant Osteopetrosis

IFN-γ is administered to subjects suffering from malignant osteopetrosis to inhibit bone overgrowth and stimulate phagocyte oxidative metabolism, thereby shifting the balance to bone resorption. Administration of IFN-γ at a dose of 1.5 micrograms per kilogram of body weight per dose three times per week for 6-18 months has been shown to enhance osteoclast function and delay disease progression by enhancing superoxide production by phagocytes. Additionally, IFN-γ treatment increases osteoclastic bone resorption in vivo in subjects with malignant osteopetrosis (Key et al. N. Engl. J. Med. 332(24):1594-9 (1995); Madyastha et al., J. Interferon Cytokine Res. 20(7):645-52 (2000)).

The modified IFN-γ proteins herein, and nucleic acids encoding modified IFN-γ polypeptides can be used in treatment of diseases or disorders in which bone overgrowth occurs, including, but not limited to malignant osteopetrosis. The modified IFN-γ polypeptides herein provide increased protein stability and improved half-life. Thus, modified IFN-γ can be used to deliver longer lasting, more stable therapies. Examples of therapeutic improvements using modified IFN-γ polypeptides include, for example, but are not limited to, lower dosages (e.g., less than 1.5 micrograms per kilogram of body weight per dose), fewer and/or less frequent administrations (e.g., fewer than 3 times per week), decreased side effects and increased therapeutic effects. Dosage can be determined empirically using the assays described herein or also, by using accepted doses of an unmodified IFN-γ as the basis for modifications to dosage regimens.

c. Arterial Conditions

IFN-γ has been shown to inhibit cholesterol accumulation and foam cell formation by down-regulating the scavenger receptor on macrophages. It also inhibits smooth muscle proliferation in culture and the formation of arterial restenosis after angioplasty in experimental animals (Hannson G K, Basic Res Cardiol. 89(Suppl 1):41-46 (1994)).

In an in vivo rat animal model, IFN-γ significantly reduced the development of neointimal hyperplasia following arterial injury (Castronuovo et al. Cardiovasc. Surg. 3(5):463-468 (1995)).

IFN-γ has been shown to significantly inhibit the expression of proliferating cell nuclear antigen (PCNA) by intimal smooth muscle cells (SMCs) in an in vivo animal model, and thus, has therapeutic use for the management of restenosis after percutaneous transluminal angioplasty (Ji et al. Chin Med J. (Engl.). 114(2): 139-142 (2001)).

Thus, the modified IFN-γ proteins herein, and nucleic acids encoding modified IFN-γ polypeptides can be used in treatment of diseases or disorders such as atherosclerosis, neointimal hyperplasia, and restenosis.

3. Chronic Granulomatous Disease (CGD)

IFN-γ is used for the prophylaxis and treatment of CGD in adults and children (Murray H W, Am. J. Med. 97(5): 459-467 (1994); Hague et al. Arch. Dis. Child. 69(4): 443-445 (1993)). Additionally, superoxide production and bacteriocidal activity of the leukocytes from some cases of chronic granulomatous disease are improved by injection of IFN-γ (Yata J. Nippon Rinsho. 50(8): 1990-5 (1992)). Administration of IFN-γ decreases the frequency of serious infections in patients with CGD. The recommended dosage of unmodified IFN-γ in CGD-afflicted children whose body surface area is greater than 0.5 sq m is 50 micrograms/sq. m. given by subcutaneous injection three times a week for life (Bolinger and Taeubel, Clin Pharm. 11(10):834-50 (1992); Ma et al. J. Formos Med. Assoc. 102(3): 189-192 (2003)).

The modified IFN-γ polypeptides herein, and nucleic acids encoding modified IFN-γ polypeptides can be used to prevent and/or treat bacterial and fungal infections associated with CGD. The modified IFN-γ polypeptides herein provide increased protein stability and improved half-life. Thus, modified IFN-γ can be used to deliver longer lasting, more stable therapies. Examples of therapeutic improvements using modified IFN-γ polypeptides include, for example, but are not limited to, lower dosages, fewer and/or less frequent administrations (e.g., less than three times per week), decreased side effects and increased therapeutic effects compared to unmodified IFN-γ. Dosage can be determined empirically using the assays described herein or also, by using accepted doses of an unmodified IFN-γ as the basis for modifications to dosage regimens.

4. Idiopathic Pulmonary Fibrosis

In idiopathic pulmonary fibrosis (IPF), the cytokine pattern of the immune response shifts toward a Th2 type response which causes lung fibrosis. IFN-γ has strong anti-fibrotic properties and administration of IFN-γ to human patients with IPF in clinical trials substantially improves the condition of the patients (Davies and Richeldi, Am. J. Respir. Med. 1(3): 211-224 (2002); Bletry and Somogyi, Ref. Med. Interne 23(Suppl 4): 511s-515s (2002); Britton, Thorax 55(Suppl 1): S37-S40 (2000)). Thus, the modified IFN-γ polypeptides herein, and nucleic acids encoding modified IFN-γ polypeptides can be used to treat fibrosis associated with IPF.

5. Hyper IgE States

a. Atopic Dermatitis

Administration of IFN-γ has been used to relieve symptoms of atopic dermatitis (Bolinger and Taeubel, Clin Pharm. 11(10):834-50 (1992)). Among its effects are a reduction in the number of CD4+ cells, reduced secretion of interleukins such as IL-4 (and thereby, IgE synthesis) and inhibition of the function of Langerhans cells. IFN-γ inhibits IgE synthesis induced by IL-4, increases expression of Fcγ receptors and increases superoxide production by circulating monocytes. Thus, host defense augmentation using IFN-γ in clinical dermatology for treatment of atopic dermatitis either alone or in combination with other agents has therapeutic value (Stadler and Ruszczak, Dermatol. Clin. 11(1): 187-199 (1993; Grassegger and Hopfl, Clin. Exp. Dermatol. 29(6): 584-588 (2004); de Prost, Y. Pediatr. Dermatol. 9(4): 386-389 (1992)). Thus, the modified IFN-γ polypeptides herein, and nucleic acids encoding modified IFN-γ polypeptides can be used to prevent and/or treat atopic dermatitis.

b. Asthma

A link between respiratory infection and asthma exacerbation has been well-established; viral respiratory tract infections are a major cause of wheezing in infants and adult asthma patients which are greater than non-asthmatics. Asthma is characterized by a strong Th2 response, and the ability of IFN-γ to switch immune responses to Th1 responses has been established. IFN-γ has therapeutic value for treating patients with asthma (Message and Johnston, Eur. Respir. J. 18: 1013-1025 (2001)). Thus, the modified IFN-γ polypeptides herein, and nucleic acids encoding modified IFN-γ polypeptides can be used to prevent and/or treat asthmatic exacerbations associated with viral infections.

c. Allergies

Allergic pathogenesis is caused by preferential activation of Th2 cells in pre-disposed individuals. The ability of IFN-γ to inhibit proliferation of Th2 cells, thereby switching the immune response to a Th1 response has been established (Wills-Karp et al. Nature Reviews Immunology 1: 69-75 (2001)). Thus, the modified IFN-γ polypeptides herein, and nucleic acids encoding modified IFN-γ polypeptides can be used to prevent and/or treat allergies.

I. Combination Therapies

Any of the modified IFN-γ polypeptides, and nucleic acid molecules encoding modified IFN-γ polypeptides described herein can be administered in combination with, prior to, intermittently with, or subsequent to, other therapeutic agents or procedures including, but not limited to, other biologics, small molecule compounds and surgery. For any disease or condition, including all those exemplified above, for which IFN-γ is indicated or has been used and for which other agents and treatments are available, IFN-γ can be used in combination therewith. Hence, the modified polypeptides provided herein similarly can be used.

For example, in viral infections, such as treatment for hepatitis, a modified IFN-γ polypeptide can be administered with additional anti-viral compounds (e.g., flavin adenine dinucleotide); or for treatment of HIV, IFN-γ can be administered with additional anti-retroviral drugs (e.g., abacavir, lamivudine and stavudine) for treatment of HIV.

As another example, a modified IFN-γ polypeptide can be used in the treatment of microbial infections with other anti-bacterial treatments such as antibiotics. For example, treatment of M. tuberculosis can include clarithromycin or rifamycin. In pulmonary tuberculosis, therapies include aerosolized administration of a modified IFN-γ polypeptide in combination with other anti-tuberculosis drugs such as the antimicrobial drug isoniazid (INH). In infected individuals, a modified IFN-γ polypeptide and INH can be administered in combination with other anti-tuberculosis drugs such as rifampin, pyrazinamide, and ethambutol, or the combination drug rifater (rifampin, isoniazid, and pyrazinamide). In another example, a modified IFN-γ polypeptide can be administered in a regimen that includes rifabutin as a first-line antibiotic for the treatment of MAC infections in combination with zidovudine, azithromycin or clarithromycin as prophylactic or therapeutic measures.

For example, in fungal infections, such as treatment for aspergillosis, a modified IFN-γ polypeptide can be administered in combination with anti-fungal agents such as dexamethasone, itroconazole, liposomal amphotericin, amphotericin B, or in combination with other cytokines such as granulocyte colony stimulating factor (G-CSF) or granulocyte macrophage colony stimulating factor (GM-CSF). A modified IFN-γ polypeptide can be administered in combination with anti-fungal agents such as nystatin and miconazole, and/or in combination with other cytokines such as granulocyte colony stimulating factor (G-CSF) or granulocyte macrophage colony stimulating factor (GM-CSF) for treatment of candidiasis. In another example, a modified IFN-γ polypeptide can be administered in combination with amphotericin B and bed rest for treatment of coccidioidomycosis and histoplasmosis. In another example, such as for treatment of cryptococcal meningitis, a modified IFN-γ polypeptide can be administered in combination with amphotericin B, slucytosine, and/or fluconazole. A modified IFN-γ polypeptide has been demonstrated to have synergistic activity with amphotericin B against intracellular C. neoformans in animal models. In another example, such as for treatment of PCP, a modified IFN-γ polypeptide can be administered in combination with pentamidine inhalations, a combination of sulfamethoxazole and trimethoprim (co-trimoxazole or TMP-SMX), trimetrexate, dapsone, primaquine, clindamycin or a combination thereof.

In another example, a modified IFN-γ polypeptide can be used in the treatment of cancers with other anti-cancer treatments such as immunotherapeutics such as ribavirun and cyclophosphamide that shift an immune response from a Th2 towards a Th1 cytokine profile, chemotherapeutic compounds, including 5-fluorouracil, cisplatin and doxorubicin, additional cytokines, inhibitors of other cytokines and/or cytokine-regulated pathways, bacillus Calmette-Guérin (BCG) and/or with other procedures such as surgery.

In malignant osteopetrosis therapy, a modified IFN-γ polypeptide can be administered in combination with other procedures including, but not limited to, bone marrow transplants.

In CGD therapy, a modified IFN-γ polypeptide can be administered in combination with other treating agents including, but not limited to, antibiotics and anti-fungal agents such as amphotericin B.

In IPF therapy, a modified IFN-γ polypeptide can be administered in combination with other treating agents including, but not limited to, glucocorticoids and corticosteroids such as Prednisone and/or other medications that suppress the body's immune system. Sometimes, immune suppressing medications such as cyclophosphamide, azathioprine, methotrexate, penicillamine and cyclosporine, or anti-inflammatory medications such as colchicine also can be used.

In asthma patients, a modified IFN-γ polypeptide can be administered in combination with other treating agents such as beta adrenergic agonists and other bronchodilating agents, including albuterol and/or corticosteroids.

In patients having allergies, a modified IFN-γ polypeptide can be administered in combination with tolerization methods with allergen peptides (i.e., allergy shots).

Visceral leishmaniasis (VL) is a severe disease associated with infection of the reticuloendothelial system by Leishmania species that is acquired through sandfly bites. VL also is an opportunistic disease in HIV-infected patients. Agents used to treat VL include, but are not limited to, organic salts of pentavalent antimony, diamidine, paromomycin, allopurinol, amphotericin B formulations and meltefosine. IFN-γ has been recognized as adjunctive treatment in at least one systemic intracellular infection: visceral leishmaniasis (Murray, H W. Intensive Care Med. 22(Suppl 4): S456-461 (1996)). IFN-γ is a potent macrophage activator with synergistic activity with antimonials in mice. Although IFN-γ has limited efficacy when administered alone to humans with VL, as an adjunctive therapy, it accerlates and/or improves the response of patients receiving antimonial therapy (Rosenthal and Marty. J. Postgrad. Med. 49: 61-68 (2003)). Thus, the modified IFN-γ polypeptides herein, and nucleic acids encoding modified IFN-γ polypeptides can be used in adjunctive therapies for visceral leishmaniasis.

The modified IFN-γ polypeptides also, optionally, can be administered with other cytokines such as G-CSF and GM-CSF, including cytokines that have been modified for increased stability. In addition, the modified IFN-γ polypeptides also, optionally, can be administered with protease inhibitors and/or other ingredients that are necessary for stabilization of unmodified and wild-type IFN-γ polypeptides upon exposure of proteases, pH and other conditions of oral delivery. For example, unmodified or modified IFN-γ polypeptides can be administered with compounds such as actinonin or epiactinonin and derivatives thereof; Bowman-Birk inhibitor and conjugates thereof; aprotinin and camostat.

J. Articles of Manufacture and Kits

Pharmaceutical compounds of modified IFN-γ polypeptides or nucleic acids encoding modified IFN-γ polypeptides, or a derivative or a biologically active portion thereof can be packaged as articles of manufacture containing packaging material, a pharmaceutical composition which is effective for treating an IFN-γ-mediated disease or disorder, and a label that indicates that modified IFN-γ polypeptide or nucleic acid molecule is to be used for treating a IFN-γ-mediated disease or disorder.

The articles of manufacture provided herein contain packaging materials. Packaging materials for use in packaging pharmaceutical products are well known to those of skill in the art. See, for example, U.S. Pat. Nos. 5,323,907, 5,052,558 and 5,033,352, each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any IFN-γ-mediated disease or disorder.

Modified IFN-γ polypeptides and nucleic acid molecules also can be provided as kits. Kits can include a pharmaceutical composition described herein and an item for administration. For example a modified IFN-γ can be supplied with a device for administration, such as a syringe, an inhaler, a dosage cup, a dropper, or an applicator. The kit can, optionally, include instructions for application including dosages, dosing regimens and instructions for modes of administration. Kits also can include a pharmaceutical composition described herein and an item for diagnosis. For example, such kits can include an item for measuring the concentration, amount or activity of IFN-γ or an IFN-γ regulated system of a subject.

### K. EXAMPLES

The following examples are included for illustrative purposes only and are not intended to limit the scope of the embodiments provided herein.

### Example 1

### Cloning cDNA Encoding a IFN-γ and Modification

A. Generation of a Vector Containing IFN-γ

To clone IFN-γ, cDNA synthesis was performed using 5 μg of Human Spleen Total RNA (BDBiosciences Clontech, Palo Alto Calif.) with SMART cDNA Synthesis kit (BDBiosciences Clontech) and the following primers (from Eurogentec, Seraing, Belgium):

SMARTSFIA:

(SEQ ID NO:362))

5′AAGCAGTGGTAACAACGCAGAGTGGCCATTATGGCCrGrGrGr3′.
OLIGODTVNSFIB:

(SEQ ID NO:363)

5′ATTCTAGAGGCCGAGGCGGCCGACATGTVN(30)3′
where V=A, G or C and N=A, G, C or T.
PCRPRIMER:

(SEQ ID NO:366)

5′AAGCAGTGGTAACAACGCAGAGT3′

IFN-γ was amplified from the synthesized cDNA by PCR with the following primers (Eurogentec):

IFGFOR [symbol] :

(SEQ ID NO:364)

5′CCC [symbol] ATGAAATATACAAGTTATATCTTGGC3′
IFGREV [symbol] :

(SEQ ID NO:365)

5′GC [symbol] TTACTGGGATGCTCTTCGACCTTGAAACAG3′

The amplified IFN-γ was cloned into the pTOPO-TA vector and confirmed by sequencing. The fragment containing IFN-γ cDNA was then removed from the pTOPO-TA vector by digestion with HindIII and XbaI and cloned into pNAUT digested with HindIII and XbaI to generate pNAUT-IFG-G1 (SEQ ID NO: 367).

B. Generation of IFN-γ Mutants

A collection of pre-designed, targeted mutants was then generated such that each individual mutant was created and processed individually, physically separated from each other and in addressable arrays. 2D-scanning technology, described herein and also described in published U.S. Application No. US-2004-0132977-A1 and U.S. application Ser. No. 10/658,355, was used to design and obtain hIFN-γ mutants with improved resistance to proteolysis and/or improved thermal tolerance. Is-HITs were identified based upon (1) the protein property to be evolved (e.g., resistance to proteolysis or stability); (2) the amino acid sequence; and (3) the properties of individual amino acids.

1. LEADS Created for Higher Resistance to Proteolysis of hIFN-γ

Variants were designed using 2D-scanning to identify positions conferring resistance to proteolysis. Positions selected (is-HITs) on hIFN-γ (SEQ ID NO: 1) were (numbering corresponds to amino acid positions in the mature protein): Y2, D5, P6, Y7, K9, E10, E12, L14, K15, K16, Y17, F18, D24, D27, N28, L31, F32, L33, L36, K37, W39, K40, E41, E42, D44, R45, F57, K58, L59, F60, K61, F63, K64, D65, D66, K71, E74, K77, E78, D79, K83, F84, K89, K90, K91, R92, D93, D94, F95, E96, K97, L98, Y101, D105, L106, E115, E122, L123, P125, K128, K131, R132, K133, R134, M137, L138, F 139, R142, and R143. The native amino acid at each of the is-HIT positions listed above was replaced by residues as shown in Table 3.

TABLE 3

| Amino acid at | Replacing |
|---|---|
| is-HIT | amino acids |
| R | H, Q |
| E | H, Q, N |
| K | Q, N |
| D | N, Q |
| M | I, V |
| P | A, S |
| Y | I, H |
| F | I, V |
| W | H, S |
| L | I, V |

Amino acids at is-HITs (left column of Table 3) were replaced by selected replacing amino acids (right column of Table 3) to produce IFN-γ variants with increased resistance to proteolysis (Table 4). In Table 4 below, the sequence identifier (SEQ ID NO:) for a IFN-γ polypeptide containing the designated substitution is in parenthesis next to each substitution.

TABLE 4
List of h IFN-γ variants for increased resistance to proteolysis

| | | | |
|---|---|---|---|
| Y2H (3) | Y2I (4) | D5N (5) | D5Q (6) |
| P6A (7) | P6S (8) | Y7H (9) | Y7I (10) |
| K9N (11) | K9Q (12) | E10Q (13) | E10H (14) |
| E10N (15) | E12Q (16) | E12H (17) | E12N (18) |
| L14I (19) | L14V (20) | K15N (21) | K15Q (22) |
| K16N (23) | K16Q (24) | Y17H (25) | Y17I (26) |
| F18I (27) | F18V (28) | D24N (29) | D24Q (30) |
| D27N (31) | D27Q (32) | N28Q (33) | N28S (34) |
| L31I (35) | L31V (36) | F32I (37) | F32V (38) |
| L33I (39) | L33V (40) | L36I (41) | L36V (42) |
| K37N (43) | K37Q (44) | W39H (45) | W39S (46) |
| K40N (47) | K40Q (48) | E41Q (49) | E41H (50) |
| E41N (51) | E42Q (52) | E42H (53) | E42N (54) |
| D44N (55) | D44Q (56) | R45H (57) | R45Q (58) |
| F57I (59) | F57V (60) | K58N (61) | K58Q (62) |
| L59I (63) | L59V (64) | F60I (65) | F60V (66) |
| K61N (67) | K61Q (68) | F63I (69) | F63V (70) |
| K64N (71) | K64Q (72) | D65N (73) | D65Q (74) |
| D66N (75) | D66Q (76) | K71N (77) | K71Q (78) |
| E74Q (79) | E74H (80) | E74N (81) | K77N (82) |
| K77Q (83) | E78Q (84) | E78H (85) | E78N (86) |
| D79N (87) | D79Q (88) | K83N (89) | K83Q (90) |
| F84I (91) | F84V (92) | K89N (93) | K89Q (94) |
| K90N (95) | K90Q (96) | K91N (97) | K91Q (98) |
| R92H (99) | R92Q (100) | D93N (101) | D93Q (102) |
| D94N (103) | D94Q (104) | F95I (105) | F95V (106) |
| E96Q (107) | E96H (108) | E96N (109) | K97N (110) |
| K97Q (111) | L98I (112) | L98V (113) | Y101H (114) |
| Y101I (115) | D105N (116) | D105Q (117) | L106I (118) |
| L106V (119) | E115Q (120) | E115H (121) | E115N (122) |
| E122Q (123) | E122H (124) | E122N (125) | L123I (126) |
| L123V (127) | P125A (128) | P125S (129) | K128N (130) |
| K128Q (131) | K131N (132) | K131Q (133) | R132H (134) |
| R132Q (135) | K133N (136) | K133Q (137) | R134H (138) |
| R134Q (139) | M137I (140) | M137V (141) | L138I (142) |
| L138V (143) | F139I (144) | F139V (145) | R142H (146) |
| R142Q (147) | R143H (148) | R143Q (149) | |

2. LEADS Created for Increased Stability of hIFN-γ (i.e. Thermal Tolerance)

Table 5 shows the list of is-HITs for increased stability (i.e., thermal tolerance) identified on hIFN-γ using 2D-scanning. Once the is-HIT target positions were selected, replacing amino acids for each is-HIT target position were identified. Appropriate replacement amino acids, specific for each is-HIT, were generated to maintain or improve the requisite activity of the polypeptide (i.e., IFN-γ cytokine activity) and increased protein stability.

Mutagenesis was performed by replacing single amino acid residues at specific is-HIT target positions one-by-one. Each mutant generated was the single product of an individual mutagenesis reaction. Substituted amino acids were compatible with protein structure and function. To select the candidate replacement amino acids for each is-HIT position, amino acid substitution matrices were used. The native amino acid at each of the is-HIT positions shown in Table 5 was replaced by residues increasing polar interaction with other amino acids: E, D, K, R, N, Q, S, T for replacing amino acids Y, A, L, S, T, I, V, F, and M, and amino acids E, D, K, R, for replacing amino acids Q and N.

The is-HIT positions identified in helices of hIFN-γ (SEQ ID NO: 1) in order to increase stability include positions that increase interactions between helices of a hIFN-γ monomer (termed intra-stability modifications), such as Helix 1 to Helix 4, Helix 2 to Helix 3, and Helix 3 to Helix 4, and interactions between helices of a hIFN-γ dimer (termed inter-stability modifications), including Helix A1 to Helix B6, Helix A2 to Helix B3, Helix A3 to Helix B5, and Helix A3 to Helix B6. In Table 5 below, the sequence identifier (SEQ ID NO:) for a IFN-γ polypeptide containing the designated substitution is in parenthesis next to each substitution.

TABLE 5
List of hIFN-γ mutants exhibiting increased stability

| | | | | | |
|---|---|---|---|---|---|
| Y7E (150) | Y7D (151) | Y7K (152) | Y7R (153) | Y7N (154) | Y7Q (155) |
| Y7S (156) | Y7T (157) | V8E (158) | V8D (159) | V8K (160) | V8R (161) |
| V8N (162) | V8Q (163) | V8S (164) | V8T (165) | A11E (166) | A11D (167) |
| A11K (168) | A11R (169) | A11N (170) | A11Q (171) | A11S (172) | A11T (173) |
| L14E (174) | L14D (175) | L14K (176) | L14R (177) | L14N (178) | L14Q (179) |
| L14S (180) | L14T (181) | Y17E (182) | Y17D (183) | Y17K (184) | Y17R (185) |
| Y17N (186) | Y17Q (187) | Y17S (188) | Y17T (189) | F18E (190) | F18D (191) |
| F18K (192) | F18R (193) | F18N (194) | F18Q (195) | F18S (196) | F18T (197) |
| I35E (198) | I35D (199) | I35K (200) | I35R (201) | I35N (202) | I35Q (203) |
| I35S (204) | I35T (205) | L36E (206) | L36D (207) | L36K (208) | L36R (209) |
| L36N (210) | L36Q (211) | L36S (212) | L36T (213) | M48E (214) | M48D (215) |
| M48K (216) | M48R (217) | M48N (218) | M48Q (219) | M48S (220) | M48T (221) |
| Q51E (222) | Q51D (223) | Q51K (224) | Q51R (225) | V53E (226) | V53D (227) |
| V53K (228) | V53R (229) | V53N (230) | V53Q (231) | V53S (232) | V53T (233) |
| S54E (234) | S54D (235) | S54K (236) | S54R (237) | S54N (238) | S54Q (239) |
| S54S (240) | S54T (241) | F55E (242) | F55D (243) | F55K (244) | F55R (245) |
| F55N (246) | F55Q (247) | F55S (248) | F55T (249) | F57E (250) | F57D (251) |
| F57K (252) | F57R (253) | F57N (254) | F57Q (255) | F57S (256) | F57T (257) |
| L59E (258) | L59D (259) | L59K (260) | L59R (261) | L59N (262) | L59Q (263) |
| L59S (264) | L59T (265) | F60E (266) | F60D (267) | F60K (268) | F60R (269) |
| F60N (270) | F60Q (271) | F60S (272) | F60T (273) | N62E (274) | N62D (275) |
| N62K (276) | N62R (277) | S72E (278) | S72D (279) | S72K (280) | S72R (281) |
| S72N (282) | S72Q (283) | S72T (284) | V73E (285) | V73D (286) | V73K (287) |
| V73R (288) | V73N (289) | V73Q (290) | V73S (291) | V73T (292) | T75E (293) |
| T75D (294) | T75K (295) | T75R (296) | T75N (297) | T75Q (298) | T75S (299) |
| I76E (300) | I76D (301) | I76K (302) | I76R (303) | I76N (304) | I76Q (305) |
| I76S (306) | I76T (307) | M80E (308) | M80D (309) | M80K (310) | M80R (311) |
| M80N (312) | M80Q (313) | M80S (314) | M80T (315) | F84E (316) | F84D (317) |
| F84K (318) | F84R (319) | F84N (320) | F84Q (321) | F84S (322) | F84T (323) |
| F95E (324) | F95D (325) | F95K (326) | F95R (327) | F95N (328) | F95Q (329) |
| F95S (330) | F95T (331) | L98E (332) | L98D (333) | L98K (334) | L98R (335) |
| L98N (336) | L98Q (337) | L98S (338) | L98T (339) | T99E (340) | T99D (341) |
| T99K (342) | T99R (343) | T99N (344) | T99Q (345) | T99S (346) | Q109E (347) |
| Q109D (348) | Q109K (349) | Q109R (350) | Q109N (351) | Q109S (352) | Q109T (353) |
| I117E (354) | I117D (355) | I117K (356) | I117R (357) | I117N (358) | I117Q (359) |
| I117S (360) | I117T (361) | | | | |

The is-HIT positions identified in helices of hIFN-γ (SEQ ID NO: 1) in order to increase stability also include positions surrounding one or more N-linked glycosylation sites at positions N28 and N100. The modifications include residues in Helix A3 to Helix B5 including: M48, Q51, S54, F55, N62, F95, L98, and T99. Residues in Helices A3 and B5 are modified such that amino acids Y, A, L, S, T, I, V, F, and M are replaced by E, D, K, R, N, Q, S, and T, and amino acids Q, N are replaced by E, D, K, and R. Modifications also include the double mutants N28K-G29P (SEQ ID NO: 368) or N28A-N100A (SEQ ID NO: 369).

### Example 2

### Production of Native and Modified IFN-γ Polypeptides (Proteins) in Mammalian Cells

Mutagenesis was performed by replacing single amino acid residues at specific is-HIT target positions one-by-one. Once replacing amino acids were identified, they were systematically introduced to replace the is-HIT loci in the protein and thus, candidate LEADs were produced. Using standard recombinant DNA methods, mutagenesis reactions were performed with the Quickchange kit (Invitrogen) using PNAUT-IFG-G1 (SEQ ID NO: 367) as the template and the presence of the mutation was verified by sequencing. Each mutant generated was the single product of an individual mutagenesis reaction. Substituted amino acids were compatible with protein structure and function.

IFN-γ was produced in Chinese Hamster Ovarian (CHO) cells (obtained from ATCC). CHO cells were cultured in F12K medium (Invitrogen) supplemented with 10% SVF and grown at 37° C. in an atmosphere of 7% CO₂. The production of native IFN-γ or variants thereof was performed by transient transfection. Cells were seeded in 6 well plates at 5×10⁵ cells/well in DMEM supplement with 1% of SVF. After 40 hours, the cells were transfected with plasmid DNA (i.e. pNAUT-IFN-γ mutants, see above) using Perfectin (GTS) in two wells per protein; two wells were mock-transfected as a negative controls for the ELISA and for activity determination. After 48 hours, the supernatants were harvested and the samples were aliquoted in 96 well plates and stored at −20° C. for standardization by ELISA (IFN-γ ELISA, R&D systems, Minneapolis Minn.) or for screening in biological activity assays as described below.

### Example 3

### Evaluation of IFN-γ Variants

For the primary screening, the collection of mutants were individually tested for two criteria in parallel: IFN-γ anti-viral activity and resistance to proteolysis. For both tests, native IFN-γ, produced and treated according to the same protocol used for the IFN-γ mutants, was used throughout the entire process as a reference (control). An international standard IFN-γ (from NIBSC; National Institute for Biological Standards and Control, Hertfordshire, United Kingdom) also was used as a second reference (control). All treatment and testing was done in triplicate.

After the ELISA for IFN-γ (see Example 2), anti-viral activity tests were performed. Serial dilutions were produced and the EC₅₀ (the concentration of IFN-γ necessary to give one-half the maximum response in an anti-viral activity assay) was obtained for each individual mutant. Two-fold serial dilutions of IFN-γ NIBSC samples were made ranging from 6000 to 2.92 pg/ml. Each sample dilution was assessed in triplicate. The anti-viral specific activity of IFN-γ (expressed as number of IU/mg of proteins) was determined based on the concentration needed for 50% protection of the cells against EMC virus-induced cytopathic effects.

For the test, HeLa cells were seeded at 2×10⁴ cells in flat-bottomed 96-well plates containing 100 μl/well of Dulbecco's MEM-GlutamaxI-sodium pyruvate medium supplemented with 10% SVF and 1 mL of gentamicin. Cells were grown at 37° C. in an atmosphere of 5% CO₂ for 24 hours. Two fold serial dilution of mutants and native IFN-γ samples in the range between 6000 and 2.92 pg/ml were prepared in 400 μl of DMEM complete media supplemented with 5% SVF into 96-well plates. Twenty-four (24) hours after seeding the cells, the medium was aspirated from each well and 100 μl of diluted serum samples was added to HeLa cells. Each sample was assessed in triplicate. The two last rows of the plates were filled with 100 μl of medium without interferon dilution samples to serve as controls for cells with (without IFN-γ) and without virus (mock cells).

After 24 hours of growth, a 1/1000 EMC virus dilution solution was placed in each well except for the cell control row (mock cells). Plates were returned to the CO₂ incubator for 40 hours. The medium was discarded, the cells were washed twice with 100 μl of PBS 1× and stained for 1 hour with 80 μl of blue staining solution containing ethanol-formamide-methyl blue mixture to determine the proportion of intact cells. Plates were washed in a distilled water bath and the cell-bound dye was extracted using 80 μl of ethylene-glycol mono-ethyl-ether. The absorbance of the dye was measured using an ELISA plate reader (Spectramax; Molecular Devices) at 660 nm. The anti-viral activity of IFN-γ samples (expressed as EC₅₀ average, pg/ml) was determined as the concentration of IFN-γ needed for 50% protection of the cells against EMC virus-induced cytopathic effects and the specific activity determined. Table 6 below depicts the specific activity (average, IU/mg) of exemplary non-limiting modified IFN-γ polypeptides and native IFN-γ.

TABLE 6
Specific activity of IFN-γ native and
variants by anti-viral activity assay

| | Specific | | Specific | | Specific |
|---|---|---|---|---|---|
| | Activity | | Activity | | Activity |
| Mutation | (IU/mg) | Mutation | (IU/mg) | Mutation | (IU/mg) |
| D5N | 1.05 × 10⁸ | D44N | nd | D94N | nd |
| D5Q | 1.29 × 10⁸ | D44Q | nd | D94Q | nd |
| P6A | 2.56 × 10⁸ | R45H | 1.03 × 10⁸ | F95I | nd |
| P6S | 1.53 × 10⁸ | R45Q | 1.98 × 10⁸ | F95V | nd |
| Y7H | 1.23 × 10⁸ | F57I | 3.14 × 10⁸ | E96Q | 1.01 × 10⁸ |
| Y7I | 1.75 × 10⁸ | F57V | 5.33 × 10⁸ | E96H | 2.44 × 10⁸ |
| K9N | 1.11 × 10⁸ | K58N | 1.00 × 10⁸ | E96N | 2.56 × 10⁸ |
| K9Q | 2.21 × 10⁸ | K58Q | 9.42 × 10⁷ | K97N | 2.22 × 10⁷ |
| E10Q | 1.92 × 10⁸ | L59I | 1.63 × 10⁷ | K97Q | 2.20 × 10⁸ |
| E10H | 2.81 × 10⁸ | L59V | 1.43 × 10⁷ | L98I | 3.80 × 10⁸ |
| E10N | 2.30 × 10⁶ | F60I | 1.63 × 10⁸ | L98V | 1.36 × 10⁷ |
| E12Q | 8.41 × 10⁷ | F60V | nd | Y101H | 2.09 × 10⁶ |
| E12H | 9.26 × 10⁷ | K61N | 2.94 × 10⁸ | Y101I | 2.21 × 10⁸ |
| E12N | 1.26 × 10⁸ | K61Q | 2.20 × 10⁸ | D105N | 2.09 × 10⁸ |
| L14I | 5.98 × 10⁷ | F63I | 3.43 × 10⁸ | D105Q | 3.27 × 10⁷ |
| L14V | 4.08 × 10⁵ | F63V | 2.75 × 10⁸ | L106I | 3.19 × 10⁸ |
| K15N | 3.49 × 10⁶ | K64N | nd | L106V | 3.56 × 10⁸ |
| K15Q | 2.30 × 10⁶ | K64Q | 3.04 × 10⁸ | E115Q | nd |
| K16N | 1.80 × 10⁸ | D65N | 1.81 × 10⁸ | E115H | nd |
| K16Q | 2.33 × 10⁸ | D65Q | 2.75 × 10⁸ | E115N | nd |
| Y17H | 3.99 × 10⁸ | D66N | 2.21 × 10⁸ | E122Q | nd |
| Y17I | 3.15 × 10⁸ | D66Q | 3.58 × 10⁸ | E122H | nd |
| F18I | 1.68 × 10⁸ | K71N | 1.59 × 10⁸ | E122N | nd |
| F18V | 7.08 × 10⁷ | K71Q | 2.02 × 10⁸ | L123I | nd |
| D24N | nd | E74Q | 7.78 × 10⁷ | L123V | nd |
| D24Q | nd | E74H | 3.94 × 10⁷ | P125A | 2.11 × 10⁸ |
| D27N | nd | E74N | 6.82 × 10⁶ | P125S | 2.14 × 10⁸ |
| D27Q | nd | K77N | 5.45 × 10⁷ | K128N | 2.76 × 10⁸ |
| L31I | nd | K77Q | 8.61 × 10⁷ | K128Q | 2.23 × 10⁸ |
| L31V | nd | E78Q | 4.07 × 10⁶ | K131N | nd |
| F32I | nd | E78H | 1.03 × 10⁶ | K131Q | nd |
| F32V | nd | E78N | 3.66 × 10⁵ | R132H | 1.73 × 10⁸ |
| L33I | 1.86 × 10⁸ | D79N | 2.40 × 10⁸ | R132Q | 2.86 × 10⁸ |
| L33V | 1.55 × 10⁸ | D79Q | nd | K133N | 2.72 × 10⁸ |
| L36I | 1.59 × 10⁷ | K83N | 6.19 × 10⁶ | K133Q | 2.56 × 10⁸ |
| L36V | 4.59 × 10⁵ | K83Q | 5.20 × 10⁷ | R134H | 3.42 × 10⁸ |
| K37N | 2.22 × 10⁸ | F84I | nd | R134Q | 2.50 × 10⁸ |
| K37Q | 1.72 × 10⁸ | F84V | nd | M137I | nd |
| W39H | nd | K89N | 4.25 × 10⁸ | M137V | 3.28 × 10⁸ |
| W39S | nd | K89Q | 4.27 × 10⁸ | L138I | nd |
| K40N | 1.90 × 10⁸ | K90N | 3.03 × 10⁸ | L138V | 3.85 × 10⁸ |
| K40Q | 2.15 × 10⁸ | K90Q | 4.23 × 10⁸ | F139I | 3.34 × 10⁸ |
| E41Q | 1.35 × 10⁸ | K91N | nd | F139V | 4.22 × 10⁸ |
| E41H | 3.53 × 10⁸ | K91Q | nd | R142H | 2.88 × 10⁸ |
| E41N | 2.37 × 10⁸ | R92H | 6.12 × 10⁷ | R142Q | 3.47 × 10⁸ |
| E42Q | 1.42 × 10⁷ | R92Q | 8.23 × 10⁷ | R143H | 1.78 × 10⁸ |
| E42H | 3.57 × 10⁷ | D93N | nd | R143Q | 2.37 × 10⁸ |
| E42N | 1.10 × 10⁷ | D93Q | nd | native | 1.25 × 10⁸ |

nd: not determined

### Example 4

### Resistance to Proteolysis Following Protease Treatment

In parallel with the determination of specific activity of IFN-γ mutants described in Example 3, the mutants were treated with protease to determine residual activity remaining after protease treatment. After the ELISA determination, up to 150 μl of supernatant containing 15 ng of native or modified IFN-γ were treated with a mixture of proteases (1% w/w of total proteins in the supernatant). The concentration of total protein in 1% of serum is 600 μg/ml based on the total amount of proteins, and not only on the amount of IFN-γ. IFN-γ mutants were treated with proteases in order to identify resistant molecules. The relative resistance of the mutant IFN-γ molecules compared to native IFN-γ was determined by exposure over time to a mixture of proteases. Protease mixtures were freshly prepared for each assay from stock solutions of endoproteinase Glu-C, 200 μg/ml; trypsin, 400 μg/ml; and α-chymotrypsin, 400 μg/ml (Sigma-Aldrich, St. Louis, Mo.). At various time points (between 5 minutes and 20 hours of incubation with protease) samples were taken and the reaction stopped by adding 10 μl of anti-protease solution (Roche). Samples were stored at −20° C. for the anti-viral activity test (see Example 3). Table 7 below depicts the residual anti-viral activity of exemplary non-limiting modified IFN-γ polypeptides and native IFN-γ following treatment with protease mixture. The resistance to proteolysis is indicated as “no change” or “increased” as compared to the residual anti-viral activity of native IFN-γ under the same protease treatment conditions. The data are not meant to be representative of all proteases, but are exemplary data showing the resistance to proteolysis to an exemplary protease cocktail containing three proteases as described above. Thus, the data are not comprehensive and are not meant to be indicative that other polypeptides do not exhibit protease resistance.

TABLE 7
Resistance to Proteolysis

| | Resistance | | Resistance | | Resistance |
|---|---|---|---|---|---|
| | to Pro- | | to Pro- | | to Pro- |
| Mutation | teolysis | Mutation | teolysis | Mutation | teolysis |
| D5N | no change | D44N | ND | D94N | ND |
| D5Q | no change | D44Q | ND | D94Q | ND |
| P6A | no change | R45H | no change | F95I | ND |
| P6S | no change | R45Q | no change | F95V | ND |
| Y7H | no change | F57I | no change | E96Q | increase |
| Y7I | no change | F57V | increase | E96H | increase |
| K9N | no change | K58N | no change | E96N | increase |
| K9Q | no change | K58Q | no change | K97N | no change |
| E10Q | no change | L59I | no change | K97Q | no change |
| E10H | no change | L59V | no change | L98I | increase |
| E10N | no change | F60I | no change | L98V | increase |
| E12Q | no change | F60V | ND | Y101H | no change |
| E12H | no change | K61N | no change | Y101I | no change |
| E12N | no change | K61Q | no change | D105N | increase |
| L14I | no change | F63I | increase | D105Q | increase |
| L14V | no change | F63V | increase | L106I | increase |
| K15N | no change | K64N | ND | L106V | increase |
| K15Q | no change | K64Q | increase | E115Q | ND |
| K16N | no change | D65N | no change | E115H | ND |
| K16Q | no change | D65Q | no change | E115N | ND |
| Y17H | no change | D66N | increase | E122Q | ND |
| Y17I | no change | D66Q | increase | E122H | ND |
| F18I | no change | K71N | no change | E122N | ND |
| F18V | no change | K71Q | no change | L123I | ND |
| D24N | ND | E74Q | no change | L123V | ND |
| D24Q | ND | E74H | no change | P125A | increase |
| D27N | ND | E74N | no change | P125S | increase |
| D27Q | ND | K77N | no change | K128N | no change |
| L31I | ND | K77Q | no change | K128Q | no change |
| L31V | ND | E78Q | no change | K131N | ND |
| F32I | ND | E78H | no change | K131Q | ND |
| F32V | ND | E78N | no change | R132H | no change |
| L33I | increase | D79N | no change | R132Q | no change |
| L33V | increase | D79Q | ND | K133N | increase |
| L36I | no change | K83N | no change | K133Q | increase |
| L36V | no change | K83Q | no change | R134H | increase |
| K37N | increase | F84I | ND | R134Q | increase |
| K37Q | increase | F84V | ND | M137I | ND |
| W39H | ND | K89N | increase | M137V | increase |
| W39S | ND | K89Q | increase | L138I | ND |
| K40N | no change | K90N | increase | L138V | increase |
| K40Q | no change | K90Q | increase | F139I | increase |
| E41Q | increase | K91N | ND | F139V | increase |
| E41H | increase | K91Q | ND | R142H | increase |
| E41N | increase | R92H | no change | R142Q | increase |
| E42Q | no change | R92Q | no change | R143H | increase |
| E42H | no change | D93N | ND | R143Q | increase |
| E42N | no change | D93Q | ND | | |

ND: Not determined

Thus, the modified IFN-γ polypeptides indicated to have an “increase” protease resistance as compared to native IFN-γ have a greater half-life and are more stable than wild-type IFN-γ polypeptides following incubation with protease.

### Example 5

### Residual Anti-Viral Activity Remaining after Protease Treatment

Anti-viral activity of human interferon gamma remaining after protease treatment at different times was determined by the capacity of the cytokine to protect HeLa cells against EMC (mouse encephalomyocarditis) virus-induced cytopathic effects (anti-viral activity) as described above in Example 3. Briefly, up to 150 μl of supernatant containing 15 ng of native or mutant IFN-γ was incubated with the protease mixture for variable times as described above in Example 4. Incubation times were (in hours): 0, 0.25, 0.5, 1, 1.5, 2, 3, 6, 10, and 20. At the appropriate time-points, 10 μl of anti-protease solution (Roche) was added to each well in order to stop proteolysis reactions. Anti-viral activity assays were then performed on each sample in order to determine the residual activity at each time-point.

To perform the anti-viral assay, HeLa cells were seeded at 2×10⁴ cells in flat-bottomed 96-well plates containing 100 μl/well of Dulbecco's MEM-GlutamaxI-sodium pyruvate medium supplemented with 10% SVF and 1 ml of gentamicin. Cells were grown at 37° C. in an atmosphere of 5% CO₂ for 24 hours. Twenty (20) μl of samples (pre-treated at different times with the protease mixtures) were diluted to 400 μl of DMEM complete media supplemented with 5% SVF into 96-well plates. Twenty-four (24) hours after seeding the cells, the medium was aspirated from each well and 100 μl of diluted serum samples were added to HeLa cells. Each sample was assessed in triplicate. The two last rows of the plates were filled with 100 μl of medium without interferon dilution samples in order to serve as controls for cells with (without IFN-γ) and without virus (mock cells).

After 24 hours of growth, a 1/1000 EMC virus dilution solution was placed in each well except for the cell control row (mock cells). Plates were returned to the CO₂ incubator for 40 hours. The medium was discarded, and the cells were washed twice with 100 μl of 1× PBS and stained for 1 hour with 80 μl of blue staining solution containing ethanol-formamide-methyl blue mixture to determine the proportion of intact cells. Plates were washed in a distilled water bath, and the cell-bound dye was extracted using 80 μl of ethylene-glycol mono-ethyl-ether. The absorbance of the dye was measured using an ELISA plate reader (Spectramax; Molecular Devices) at 660 nm and an OD (optical density) value determined.

As an internal control IFN-γ provided by NIBSC (National Institute for Biological Standards and Control, Hertfordshire United Kingdom) was applied to each assay in order to standardize the assay. Two-fold serial dilutions of NIBSC IFN-γ samples were made ranging from 6000 to 2.92 pg/ml. Each sample dilution was assessed in triplicate.

IFN-γ mutants R142Q, E41N, F57V, M137I, L138I, L33V, E41Q, D65Q and L98I and wildtype IFN-γ were tested for their residual anti-viral activity following incubation with protease mixture at different time points. The anti-viral activity of wildtype IFN-γ (depicted as QD value) was set at 100 in the absence of incubation with protease. Following incubation with protease mixture for 0.25 hours to 0.5 hours, the anti-viral activity of wildtype IFN-γ decreased 5-fold, and no detectable residual activity was observed for wildtype IFN-γ following incubation with protease for 1 hour or more. All mutants tested showed an increased anti-viral activity half-life in vitro compared to wildtype IFN-γ. For example, all mutants tested showed little to no loss in anti-viral activity following incubation with protease for 0.5 hours. All mutants tested showed some anti-viral activity after 6 hour incubation with protease, with some mutants exhibiting anti-viral activity following incubation with protease for 10 or more hours.

### Example 6

### Determination of Anti-Proliferative Activity

Anti-proliferative activity of IFN-γ was determined by assessing the capacity of the cytokine to inhibit proliferation of Daudi cells. Daudi cells (1×10⁴ cells) were seeded in flat-bottomed 96-well plates containing 50 μl/well of RPMI-1640 medium supplemented with 10% SVF, 1× glutamine and 1 mL of gentamicin. No cells were added to the last row (“H” row) of the flat-bottomed 96-well plates in order to evaluate background absorbance of culture medium.

At the same time, two-fold serial dilutions of IFN-γ samples were made with RPMI 1640 complete medium into 96-Deep-Well plates with final concentration ranging from 6000 to 2.9 pg/ml. IFN-γ dilutions (50 μl) were added to each well containing 50 μl of Daudi cells for a total volume in each well of 100 μl. Each IFN-γ sample dilution was assessed in triplicate. Each well of the “G” row of the plates was filled with 50 μl of RPMI 1640 complete medium as a positive control. The plates were incubated for 72 hours at 37° C. in a humidified, 5% CO₂ atmosphere.

After 72 hours of growth, 20 μl of Cell titer 96 Aqueous one solution reagent (Promega) was added to each well and incubated for 1.5 hours at 37° C. in an atmosphere of 5% CO₂. To measure the amount of colored soluble formazan produced by cellular reduction of the MTS, the absorbance of the dye was measured using an ELISA plate reader (Spectramax) at 490 nm.

The corrected absorbances (“H” row background value subtracted) obtained at 490 nm were plotted versus concentration of cytokine. The ED₅₀ value was calculated by determining the X-axis value corresponding to one-half the difference between the maximum and minimum absorbance values. (ED₅₀=the concentration of cytokine necessary to give one-half the maximum response).

### Example 7

### Thermal Tolerance Assay

An ELISA determination was performed to assess protein concentration. Up to 150 μl of supernatant containing 1 ng of native IFN-γ or IFN-γ variants was used for the thermal tolerance assay. The supernatant was incubated at 37° C. over a 60 hour period in DMEM (Invitrogen) and anti-protease mixture (Roche) in a final reaction volume of 1200 μl. At each times point, an aliquot of 100 μl was taken, and frozen and stored at −20° C. for the activity test (anti-viral activity; see Example 3). Time points for analysis included 0, 2, 4, 8, 12, 20, 28, 40, 48, and/or 60 hours of incubation.

Since modifications will be apparent to those of skill in this art, it is intended that this invention be limited only by the scope of the appended claims.

1. A modified interferon-γ polypeptide, comprising one or more amino acid replacements in an unmodified IFN-γ polypeptide at positions corresponding to any of amino acid positions 2, 5-12, 14-18, 24, 27-29, 31, 32, 35, 36, 39, 44, 45, 48, 51, 53-55, 57, 59, 60, 62, 63, 71-80, 83, 84, 89-99, 100, 101, 105, 106, 109, 115, 117, 122, 123, 125, 128, 131-134, 137-139, 142, 143 of a mature interferon-γ, wherein:
the mature human interferon-γ comprises the sequence of amino acids set forth in SEQ ID NO: 1;
the modified interferon-γ polypeptide exhibits increased protein stability compared to the unmodified human interferon-γ ; and
if position 5 is replaced, the replacing amino acid is not cysteine;
if position 6, 12, 17, 24, 62, 71, 74, 75, 77, 78, 89, 93, 96, 105, or 106 is replaced, the replacing amino acid is not cysteine;
if position 9 or 28 is replaced, the replacing amino acid is not glutamine or cysteine;
if position 15, 83 or 90 is replaced, the replacing amino acid is not serine, cysteine, or threonine;
if position 29 is replaced, the replacing amino acid is not phenylalanine, asparagine, tyrosine, glutamine, valine, alanine, methionine, isoleucine, lysine, arginine, threonine, histidine, cysteine, or serine;
if position 31 is replaced, the replacing amino acid is not histidine, aspartic acid, alanine, methionine, asparagine, threonine, arginine, serine, or cysteine;
if position 18, 32, 55, 57, 60, 63, 84, 95, or 139 is replaced, the replacing amino acid is not valine;
if position 48, 73, or 143 is replaced, the replacing amino acid is not asparagine;
if position 97 or 122 is replaced, the replacing amino acid is not asparagine or cysteine;
if position 100 is replaced, the replacing amino acid is not glutamine;
if position 101 is replaced, the replacing amino acid is not phenylalanine, asparagine, glutamine, valine, alanine, methionine, isoleucine, lysine, glycine, arginine, threonine, histidine, cysteine, or serine;
if position 109 is replaced, the replacing amino acid is not serine or threonine; and
if position 133 is replaced, the replacing amino acid is not asparagine.

2. The modified interferon-γ polypeptide of claim 1, wherein the replacements in an unmodified IFN-γ polypeptide are at positions corresponding to any of amino acid positions 2, 7, 8, 10, 11, 14, 16, 27, 35, 36, 39, 44, 45, 51, 53, 54, 59, 72, 76, 80, 91, 92, 94, 98, 115, 117, 123, 125, 128, 131, 132, 134, 137, 138 and 142.

3. The modified interferon-γ of claim 1, wherein:
the amino acid replacement or replacements correspond to positions selected from among positions Y2, D5, P6, Y7, V8, K9, E10, A11, E12, L14, K15, K16, Y17, F18, D24, D27, N28, G29, L31, F32, I35, L36, W39, D44, R45, M48, Q51, V53, S54, F55, F57, L59, F60, N62, F63, K71, S72, V73, E74, T75, I76, K77, E78, D79, M80, K83, F84, K89, K90, K91, R92, D93, D94, F95, E96, K97, L98, T99, N100, Y101, D105, L106, Q109, E115, I117, E122, L123, P125, K128, K131, R132, K133, R134, M137, L138, F139, R142 and R143 of the mature human interferon-γ polypeptide.

4. The modified interferon-γ of claim 1, wherein the amino acid replacement or replacements are selected from among Y2H, Y2I, D5N, D5Q, P6A, P6S, Y7H, Y7I, Y7E, Y7D, Y7K, Y7R, Y7N, Y7Q, Y7S, Y7T, V8E, V8D, V8K, V8R, V8N, V8Q, V8S, V8T, K9N, E10Q, E10H, E10N, A11E, A11D, A11K, A11R, A11N, A11Q, A11S, A11T, E12Q, E12H, E12N, L14I, L14V, L14E, L14D, L14K, L14R, L14N, L14Q, L14S, L14T, K15N, K15Q, K16N, K16Q, Y17H, Y17I, Y17E, Y17D, Y17K, Y17R, Y17N, Y17Q, Y17S, Y17T, F18I, F18E, F18D, F18K, F18R, F18N, F18Q, F18S, F18T, D24N, D24Q, D27N, D27Q, N28S, G29P, L31I, L31V, F32I, I35E, I35D, I35K, I35R, I35N, I35Q, I35S, I35T, L36I, L36V, L36E, L36D, L36K, L36R, L36N, L36Q, L36S, L36T, W39H, W39S, D44N, D44Q, R45H, R45Q, M48E, M48D, M48K, M48R, M48Q, M48S, M48T, Q51E, Q51D, Q51K, Q51R, V53E, V53D, V53K, V53R, V53N, V53Q, V53S, V53T, S54E, S54D, S54K, S54R, S54N, S54Q, S54T, F55E, F55D, F55K, F55R, F55N, F55Q, F55S, F55T, F57I, F57E, F57D, F57K, F57R, F57N, F57Q, F57S, F57T, L59I, L59V, L59E, L59D, L59K, L59R, L59N, L59Q, L59S, L59T, F60I, F60E, F60D, F60K, F60R, F60N, F60Q, F60S, F60T, N62E, N62D, N62K, N62R, F63I, K71N, K71Q, S72E, S72D, S72K, S72R, S72N, S72Q, S72T, V73E, V73D, V73K, V73R, V73Q, V73S, V73T, E74Q, E74H, E74N, T75E, T75D, T75K, T75R, T75N, T75Q, T75S, I76E, I76D, I76K, I76R, I76N, I76Q, I76S, I76T, K77N, K77Q, E78Q, E78H, E78N, D79N, D79Q, M80E, M80D, M80K, M80R, M80N, M80Q, M80S, M80T, K83N, K83Q, F84I, F84E, F84D, F84K, F84R, F84N, F84Q, F84S, F84T, K89N, K89Q, K90N, K90Q, K91N, K91Q, R92H, R92Q, D93N, D93Q, D94N, D94Q, F95I, F95E, F95D, F95K, F95R, F95N, F95Q, F95S, F95T, E96Q, E96H, E96N, K97Q, L98I, L98V, L98E, L98D, L98K, L98R, L98N, L98Q, L98S, L98T, T99E, T99D, T99K, T99R, T99N, T99Q, T99S, N100A, D105N, D105Q, L106I, L106V, Q109E, Q109D, Q109K, Q109R, Q109N, E115Q, E115H, E115N, I117E, I117D, I117K, I117R, I117N, I117Q, I117S, I117T, E122Q, E122H, L123I, L123V, P125A, P125S, K128N, K128Q, K131N, K131Q, R132H, R132Q, K133Q, R134H, R134Q, M137I, M137V, L138I, L138V, F139I, R142H, R142Q, R143H, and R143Q.

5. The modified interferon-γ of claim 4, wherein:
the amino acid replacement or replacements is (are) selected from among Y2H, Y2I, D5N, D5Q, P6A, P6S, Y7H, Y7I, K9N, E10Q, E10H, E10N, E12Q, E12H, E12N, L14I, L14V, K15N, K15Q, K16N, K16Q, Y17H, Y17I, F18I, D24N, D24Q, D27N, D27Q, N28S, L31I, L31V, F32I, L36I, L36V, W39H, W39S, D44N, D44Q, R45H, R45Q, F57I, L59I, L59V, F60I, F63I, K71N, K71Q, E74Q, E74H, E74N, K77N, K77Q, E78Q, E78H, E78N, D79N, D79Q, K83N, K83Q, F84I, K89N, K89Q, K90N, K90Q, K91N, K91Q, R92H, R92Q, D93N, D93Q, D94N, D94Q, F95I, E96Q, E96H, E96N, K97Q, L98I, L98V, D105N, D105Q, L106I, L106V, E115Q, E115H, E115N, E122Q, E122H, L123I, L123V, P125A, P125S, K128N, K128Q, K131N, K131Q, R132H, R132Q, K133Q, R134H, R134Q, M137I, M137V, L138I, L138V, F139I, R142H, R142Q, R143H, and R143Q; and
the modified polypeptide exhibits increased resistance to proteolysis.

6. The modified interferon-γ of claim 5, wherein the amino acid replacement or replacements is (are) selected from among P6A, P6S, Y7I, E10Q, E10H, K16Q, Y17H, Y17I, F18I, F60I, F63I, K71N, K71Q, D79N, K89N, K89Q, K90N, K90Q, E96H, E96N, K97Q, L98I, D105N, L106I, L106V, P125A, P125S, K128N, K128Q, R132H, R132Q, K133Q, R134H, R134Q, M137I, M137V, L138I, L138V, F139I, F139V, R142H, R142Q, R143H, and R143Q.

7. The modified interferon-γ of claim 5, wherein the amino acid replacement or replacements is (are) selected from among F63I, K89N, K89Q, K90N, K90Q, E96Q, E96H, E96N, L98I, L98V, D105N, D105Q, L106I, L106V, P125A, P125S, K133Q, R134H, R134Q, M137V, L138V, F139I, R142H, R142Q, R143H, and R143Q.

8. The modified interferon-γ of claim 5, wherein the amino acid replacement or replacements is (are) selected from among F63I, K89N, K89Q, K90N, E96H, E96N, L98I, D105N, L106I, L106V, P125A, P125S, K133Q, R134H, R134Q, M137V, L138V, F139I, F139V, R142H, R142Q, R143H, and R143Q.

9. The modified interferon-γ polypeptide of claim 4, wherein:
the amino acid replacement or replacements is (are) selected from among Y7E, Y7D, Y7K, Y7R, Y7N, Y7Q, Y7S, Y7T, V8E, V8D, V8K, V8R, V8N, V8Q, V8S, V8T, A11E, A11D, A11K, A11R, A11N, A11Q, A11S, A11T, L14E, L14D, L14K, L14R, L14N, L14Q, L14S, L14T, Y17E, Y17D, Y17K, Y17R, Y17N, Y17Q, Y17S, Y17T, F18E, F18D, F18K, F18R, F18N, F18Q, F18S, F18T, I35E, I35D, I35K, I35R, I35N, I35Q, I35S, I35T, L36E, L36D, L36K, L36R, L36N, L36Q, L36S, L36T, M48E, M48D, M48K, M48R, M48Q, M48S, M48T, Q51E, Q51D, Q51K, Q51R, V53E, V53D, V53K, V53R, V53N, V53Q, V53S, V53T, S54E, S54D, S54K, S54R, S54N, S54Q, S54T, F55E, F55D, F55K, F55R, F55N, F55Q, F55S, F55T, F57E, F57D, F57K, F57R, F57N, F57Q, F57S, F57T, L59E, L59D, L59K, L59R, L59N, L59Q, L59S, L59T, F60E, F60D, F60K, F60R, F60N, F60Q, F60S, F60T, N62E, N62D, N62K, N62R, S72E, S72D, S72K, S72R, S72N, S72Q, S72T, V73E, V73D, V73K, V73R, V73Q, V73S, V73T, T75E, T75D, T75K, T75R, T75N, T75Q, T75S, I76E, I76D, I76K, I76R, I76N, I76Q, I76S, I76T, M80E, M80D, M80K, M80R, M80N, M80Q, M80S, M80T, F84E, F84D, F84K, F84R, F84N, F84Q, F84S, F84T, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, T99S, Q109E, Q109D, Q109K, Q109R, I117E, I117D, I117K, I117R, I117N, I117Q, I117S and I117T.

10. The modified interferon-γ polypeptide of claim 9, wherein:
the amino acid replacement or replacements is (are) selected from among Y7E, Y7D, Y7K, Y7R, Y7N, Y7Q, Y7S, Y7T, A11E, A11D, A11K, A11R, A11N, A11Q, A11S, A11T, L14E, L14D, L14K, L14R, L14N, L14Q, L14S, L14T, Y17E, Y17D, Y17K, Y17R, Y17N, Y17Q, Y17S, Y17T, L36E, L36D, L36K, L36R, L36N, L36Q, L36S, L36T, V53E, V53D, V53K, V53R, V53N, V53Q, V53S, V53T, F57E, F57D, F57K, F57R, F57N, F57Q, F57S, F57T, F60E, F60D, F60K, F60R, F60N, F60Q, F60S, F60T, S72E, S72D, S72K, S72R, S72N, S72Q, S72T, V73E, V73D, V73K, V73R, V73Q, V73S, V73T, T75E, T75D, T75K, T75R, T75N, T75Q, T75S, I76E, I76D, I76K, I76R, I76N, I76Q, I76S, I76T, M80E, M80D, M80K, M80R, M80N, M80Q, M80S, M80T, F84E, F84D, F84K, F84R, F84N, F84Q, F84S, and F84T; and
the modified polypeptide exhibits increased intra-stability of the polypeptide monomer.

11. The modified interferon-γ polypeptide of claim 9, wherein:
the amino acid replacement or replacements is (are) selected from among Y7E, Y7D, Y7K, Y7R, Y7N, Y7Q, Y7S, Y7T, V8E, V8D, V8K, V8R, V8N, V8Q, V8S, V8T, A11E, A11D, A11K, A11R, A11N, A11Q, A11S, A11T, L14E, L14D, L14K, L14R, L14N, L14Q, L14S, L14T, F18E, F18D, F18K, F18R, F18N, F18Q, F18S, F18T, I35E, I35D, I35K, I35R, I35N, I35Q, I35S, I35T, M48E, M48D, M48K, M48R, M48N, M48Q, M48S, M48T, Q51E, Q51D, Q51K, Q51R, S54E, S54D, S54K, S54R, S54N, S54Q, S54T, F55E, F55D, F55K, F55R, F55N, F55Q, F55S, F55T, L59E, L59D, L59K, L59R, L59N, L59Q, L59S, L59T, N62E, N62D, N62K, N62R, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, T99S, Q109E, Q109D, Q109K, Q109R, I117E, I117D, I117K, I117R, I117N, I117Q, I117S and I117T; and
the modified polypeptide exhibits increased inter-stability compared to an unmodified IFN-γ polypeptide.

12. The modified interferon-γ polypeptide of claim 4, wherein:
the amino acid replacement or replacements is (are) selected from among N28A, N28K, G29P, M48E, M48D, M48K, M48R, M48N, M48Q, M48S, M48T, Q51E, Q51D, Q51K, Q51R, S54E, S54D, S54K, S54R, S54N, S54Q, S54T, F55E, F55D, F55K, F55R, F55N, F55Q, F55S, F55T, N62E, N62D, N62K, N62R, F95E, F95D, F95K, F95R, F95N, F95Q, F95S, F95T, L98E, L98D, L98K, L98R, L98N, L98Q, L98S, L98T, T99E, T99D, T99K, T99R, T99N, T99Q, T99S, and N100A; and
the modified interferon-γ polypeptide exhibits increased stability around glycosylation sites.

13. The modified interferon-γ polypeptide of claim 12, wherein the amino acid replacements are N28A and N100A.

14. The modified interferon-γ polypeptide of claim 12, wherein the amino acid replacements are N28K and G29P.

15. A modified human interferon-γ polypeptide, comprising two or more amino acid replacements in an unmodified IFN-γ polypeptide at positions corresponding to any of amino acid positions of a mature interferon-γ polypeptide, wherein:
the mature human interferon-γ comprises the sequence of amino acids set forth in SEQ ID NO: 1; and
the modified interferon-γ exhibits increased protein stability compared to the unmodified interferon-γ polypeptide.

16. The modified interferon-γ polypeptide of claim 15, that comprises 2, 3, 4, 5, 6, 7, 8, 9, 10, 11, 12, 13, 14, 15, 16, 17, 18, 19 or 20 amino acid replacements in an unmodified interferon-γ polypeptide.

17. The modified interferon-γ polypeptide of claim 1 or claim 15, wherein the modified interferon-γ further comprises a modification in one or more of positions corresponding to positions 5, 9, 28, 33, 37, 40, 41, 42, 58, 61, 64-66, 86, 88, 124, 127, 128, 133 and 140 in a mature interferon-γ polypeptide having a sequence of amino acids set forth in SEQ ID NO: 1.

18. The modified interferon-γ of claim 17, wherein the further modifications are selected from among any one or more of amino acid replacements corresponding to D5N, K9Q, N28S, N28A, N28H, L33I, L33V, K37N, K37Q, K40N, K40Q, E41H, E41N, E41Q, E42Q, E42H, E42N, K58N, K58Q, K61N, K61Q, K64N, K64Q, D65N, D65Q, D66N, D66Q, N86D, N88D, S124P, K128E, K133T and Q140R.

19. The modified interferon-γ of claim 1 or claim 15, wherein the unmodified interferon-γ polypeptide has a sequence of amino acids set forth in SEQ ID NO:1 or SEQ ID NO:2.

20. The modified interferon-γ of claim 1 or claim 15 that is a human interferon-γ.

21. The modified interferon-γ of claim 1 that has a length of 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169 or 170 amino acids, or combinations thereof.

22. The modified interferon-γ of claim 15 that has a length of 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169 or 170 amino acids, or combinations thereof.

23. The modified interferon-γ of claim 1 that is 132, 143 or 146 amino acids in length.

24. The modified interferon-γ of claim 15 that is 132, 143 or 146 amino acids in length.

25. The modified interferon-γ of claim 1 or claim 15, wherein the one or more amino acid replacements are selected from among natural amino acids, non-natural amino acids and a combination of natural and non-natural amino acids.

26. The modified interferon-γ polypeptide of claim 1, wherein only the primary sequence is modified and the polypeptide exhibits increased protein stability.

27. The modified interferon-γ polypeptide of claim 15, wherein only the primary sequence is modified and the polypeptide exhibits increased protein stability.

28. The modified interferon-γ of claim 1, wherein the interferon-γ is pegylated, albuminated, or glycosylated.

29. The modified interferon-γ of claim 15, wherein the interferon-γ is pegylated, albuminated, or glycosylated.

30. The modified interferon-γ of claim 1 or claim 15, wherein increased stability is manifested as increased resistance to proteolysis.

31. The modified interferon-γ of claim 30, wherein the increased resistance to proteolysis occurs in serum, blood, saliva, digestive fluids or in vitro when exposed to one or more proteases.

32. The modified interferon-γ of claim 30, wherein the increased resistance to proteolysis is exhibited by the modified interferon-γ when it is administered orally or is present in the digestive tract.

33. The modified interferon-γ of claim 31, wherein the one or more proteases is selected from among trypsin, trypsin (Arg blocked), trypsin (Lys blocked), clostripain, endoproteinase Asp-N, chymotrypsin, cyanogen bromide, iodozobenzoate, Myxobacter P., Armillaria, luminal pepsin, microvillar endopeptidase, dipeptidyl peptidase, enteropeptidase, hydrolase, pepsin, elastase, aminopeptidase, gelatinase B, gelatinase A, α-chymotrypsin, carboxypeptidase, endoproteinase Arg-C, endoproteinase Asp-N, endoproteinase Glu-C, endoproteinase Lys-C, NS3, elastase, factor Xa, Granzyme B, thrombin, plasmin, urokinase, tPA and PSA.

34. The modified interferon-γ of claim 1, wherein the increased stability is manifested as increased thermal tolerance.

35. The modified interferon-γ of claim 15, wherein the increased stability is manifested as increased thermal tolerance.

36. The modified interferon-γ of claim 1 or claim 15, wherein the increased stability is manifested as an increased half-life in vivo or in vitro or as an increased half-life when administered to a subject.

37. The modified interferon-γ of claim 1 or claim 15 that comprises a signal peptide.

38. The modified interferon-γ of claim 37, wherein the signal peptide is amino acids 1-20 of the sequence of amino acids set forth in SEQ ID NO: 370 or 371.

39. The modified interferon-γ of claim 1 or claim 15 that is produced in mammalian cells or in insect cells.

40. The modified interferon-γ of claim 1 or claim 15 that is produced in E. coli.

41. The modified interferon-γ of claim 1 that has a single mutation compared to the unmodified interferon-γ, wherein the unmodified is a native interferon-γ.

42. The modified interferon-γ of claim 1 or claim 15 that has 2, 3, 4, 5, 6, 7, 8, 9, 10, 11, 12, 13, 14, 15, 16, 17, 18, 19, or 20 mutations compared to the unmodified interferon-γ, wherein the unmodified is a native interferon-γ.

43. The modified interferon-γ of claim 1 or claim 15 that further contains mutations to optimize one or more glycosylation sites.

44. The modified interferon-γ of claim 1 or claim 15 that is a dimer, wherein the dimer is either a homodimer or a heterodimer, comprising interferon-γ.

45. The modified interferon-γ of claim 44, wherein the dimer is a fusion protein, and wherein the two interferon-γ monomers are linked directly or indirectly via linker, via covalent or via non-covalent linkages.

46. The modified interferon-γ of claim 1 or claim 15 that is monomer.

47. A modified interferon-γ, comprising any of the sequences of amino acids set forth in SEQ ID NOS: 3-11, 13-27, 29-32, 34-37, 41, 42, 45, 46, 55-59, 63-65, 69, 77-91, 93-105, 107-109, 111-113, 116-124, 126-135, 137-144, 146-217, 219-239, 241-350, 354-361, 368 and 369, or an active portion thereof.

48. A library, comprising two, three, four, five, six, 10, 50, 100, or more modified interferon-γ polypeptides of claim 1 or claim 15.

49. A nucleic acid molecule, comprising a sequence of nucleic acids encoding a modified interferon-γ polypeptide of claim 1 or claim 15.

50. A vector, comprising the nucleic acid molecule of claim 49.

51. A eukaryotic cell, comprising the vector of claim 50.

52. A prokaryotic cell, comprising the vector of claim 50.

53. A method for expressing a modified interferon-γ, comprising:
i) introducing a vector of claim 50 into a cell; and
ii) culturing the cell under conditions in which the encoded modified interferon-γ polypeptide is expressed.

54. A pharmaceutical composition, comprising the modified interferon-γ of claim 1 or claim 15 in a pharmaceutically acceptable excipient.

55. The pharmaceutical composition of claim 54, wherein the pharmaceutical composition is formulated for oral, nasal or pulmonary administration.

56. The pharmaceutical composition of claim 54, wherein the pharmaceutical composition is formulated for oral administration.

57. The pharmaceutical composition of claim 56, wherein the pharmaceutical composition does not contain exogenously added protease inhibitors.

58. The pharmaceutical composition of claim 56, wherein the pharmaceutical composition is formulated for administration in a form selected from among a liquid, a pill, a tablet and a capsule.

59. A pharmaceutical composition, comprising a nucleic acid molecule of claim 49 in a pharmaceutically acceptable excipient.

60. A method, comprising administering a pharmaceutical composition of claim 54 to a subject who has a disease or condition that is treated by administration of interferon-γ.

61. The method of claim 60, wherein the disease or condition is selected from among a viral infection, a bacterial infection, a fungal infection, a protozoa infection, a cancer or a cancer-associated condition, idiopathic pulmonary fibrosis, a fibrotic condition, a hyper-IgE condition, malignant osteopetrosis and chronic granulomatous.

62. The method of claim 61, wherein the viral infection is hepatitis C or human immunodeficiency virus (HIV).

63. The method of claim 61, wherein the cancer-associated condition is neutropenia or hemopoietic cell transplantation, or the fungal infection is aspergillosis or candidemia.

64. A method, comprising administering a pharmaceutical composition of claim 56 to a subject who has a disease or condition that is treated by administration of interferon-γ.

65. The method of claim 64, wherein the disease or condition is selected from among a viral infection, a bacterial infection, a fungal infection, a protozoa infection, a cancer or a cancer-associated condition, idiopathic pulmonary fibrosis, a fibrotic condition, a hyper-IgE condition, malignant osteopetrosis and chronic granulomatous.

66. The method of claim 65, wherein the viral infection is hepatitis C or human immunodeficiency virus (HIV).

67. The method of claim 65, wherein the cancer-associated condition is neutropenia or hemopoietic cell transplantation, or the fungal infection is aspergillosis or candidemia.
